(19) United States
(12) Patent Application Publication
(10) Pub. No.: US 2024/0228986 A1
(43) Pub. Date: Jul. 11, 2024

(54) ENGINEERED CELLS, ENZYMES, AND METHODS FOR PRODUCING CANNABINOIDS

(71) Applicant: Cellibre, Inc., San Diego, CA (US)

(72) Inventors: Spiros Kambourakis, San Diego, CA (US); Nicholas Donald Keul, San Diego, CA (US); Russell Scott Komor, San Diego, CA (US); Jun Urano, Irvine, CA (US); Nicky Christopher Caiazza, Encinitas, CA (US)

(21) Appl. No.: 18/560,883

(22) PCT Filed: May 13, 2022
(86) PCT No.: PCT/US2022/029326

§ 371 Date: Nov. 14, 2023

Related U.S. Application Data
(60) Provisional application No. 63/188,648, filed on May 14, 2021.

(51) Int. Cl. C12N 9/10; C12P 7/22

(52) U.S. Cl. CPC C12N 9/1085; C12P 7/22; C12Y 205/01001; C07K 2319/00

(57) ABSTRACT

Disclosed herein are novel CBGA and CBGVA synthases and methods for improvement of their overall activities for the synthesis of CBGA and CBGVA from their respective precursors, olivetolic acid (OA) or divarinic acid (DVA) and GPP. Also disclosed are fusion proteins to enhance synthesis of CBGA and CBGVA. The methods described herein also increase the titer and the purity of CBGA and CBGVA made by a cell by 1) decreasing the formation of byproducts FCBGA and FCBGVA that are synthesized from the respective prenylation of OA and DVA with FPP, and/or 2) increasing the intracellular availability of OA and DVA.

### RELATED APPLICATION

This application claims priority to, and the benefit of, co-pending U.S. Provisional Application No. 63/188,648, filed May 14, 2021. The disclosure of said provisional application is hereby incorporated by reference in its entirety.

### BACKGROUND OF THE INVENTION

The Cannabaceae family of plants produces numerous different cannabinoids (>=120) in variable, relative quantities over a 7-10 week flowering period. Many of these cannabinoids have been and are currently being explored as therapeutics in chordates (e.g., mammals), and as a result, they are largely approved for medical and/or recreational use in the United States (Abrams D I Eur J Int Med 2018, 49, 7-11). Specifically, the most sought after (phyto)cannabinoids are: tetrahydrocannabinolic acid (THCA), cannabidiolic acid (CBDA), and cannabichromenic acid (CBCA). These phytocannabinoids and their associated chemical analogs are all biosynthesized in various quantities from the same pre-cursor: cannabigerolic acid (CBGA). Thus, to mass produce any specific phytocannabinoid (e.g., THCA/CBDA/CBCA/etc), both the rate and total quantity of biosynthesized CBGA must be enhanced and increased.

### SUMMARY OF THE INVENTION

Described herein are novel CBGA and cannabigerovarinic acid (CBGVA) synthases and methods for improvement of their overall activities for the synthesis of CBGA and CBGVA from their respective precursors, olivetolic acid (OA) or divarinic acid (DVA) and geranyl pyrophosphate (GPP). In some embodiments, methods described herein also increase the titer and the purity of CBGA and CBGVA made by a cell by 1) decreasing the formation of byproducts farnesyl cannabigerolic acid (FCBGA) and farnesyl cannabigerovarinic acid (FCBGVA) that are synthesized from the respective prenylation of OA and DVA with farnesyl pyrophosphate (FPP) (e.g., FIGS. 1 and/or 2) increasing the intracellular availability of OA and DVA. The latter can be achieved by over expressing native or exogenous aromatic acid importers and/or the inactivation of native exporter proteins. Improvement of prenyltransferase activity and selectivity is also achieved by fusions of CBGA synthases with other enzymes such as GPP synthases and their mutants. Additionally, by providing a fusion protein which contains a polyketide cyclase (PKC) in addition to a prenyltransferase and a GPP synthase, the flux of OA/DVA made from hexanoic or butyric acid is increased, resulting in an overall increase in CBGA production. Thus, the present invention pertains, in some embodiments, to a fusion comprising two enzymes, GPP synthase and prenyl transferase (soluble or membrane bound), as well as a fusion comprising three enzymes, GPP synthase, polyketide cyclase and prenyl transferase to effectuate an increased titer and purity of CBGA and CBGVA.

Some aspects of the present disclosure are directed to a recombinant membrane-bound prenyltransferase (rMPT), the rMPT comprising an amino acid sequence having at least one amino acid modification as compared to a naturally occurring membrane-bound prenyltransferase. In some embodiments, the rMPT comprises an amino acid sequence with at least 70% identity to SEQ ID NO: 1 (MPT1) or SEQ ID NO: 2 (MPT4) or SEQ ID 52 (MPT4.1). In some embodiments, the rMPT comprises an amino acid sequence comprising portions of the amino acid sequence of SEQ ID NO: 1 (MPT1) and SEQ ID NO: 2 (MPT4) or SEQ ID 52 (MPT4.1). In some embodiments, the rMPT comprises a functional fragment of MPT4 (SEQ ID NO: 2). In some embodiments, the rMPT comprises an amino acid sequence with at least 90% identity to SEQ ID NO: 22 (MPT21), SEQ ID NO: 23 (MPT26), or SEQ ID NO: 24 (MPT31).

In some embodiments, the rMPT comprises an amino acid sequence having at least one amino acid modification as compared to SEQ ID NO: 22 (MPT21). In some embodiments, the at least one amino acid modification is a deletion, substitution or insertion at an amino acid position selected from R22, P23, Y24, V25, V26, K27, G28, M29, S31, A33, F66, N67, A70, A71, D80, I81, I84, N85, K86, P87, D88, L89, L91, V92, Y139, S140, F154, L155, I158, S159, S160, V162, T194, V195, G197, M198, I200, A201, F202, A203, K204, D208, I209, G211, D212, L267, A268, L271, T275, L278, N282, A284, S285, R289, F292, I295, W296, L298, and Y299.

In some embodiments, the rMPT comprises an amino acid sequence having at least one amino acid modification as compared to SEQ ID NO: 2 (MPT4) or SEQ ID 52 (MPT4.1). In some embodiments, the at least one amino acid modification is a deletion, substitution or insertion at an amino acid position selected from R22, P23, Y24, V25, V26, K27, G28, M29, S31, A33, F66, N67, A70, A71, D80, I81, I84, N85, K86, P87, D88, L89, L91, V92, Y139, S140, F154, L155, I158, S159, S160, V162, T194, V195, G197, M198, I200, A201, F202, A203, K204, D208, I209, G211, D212, L267, A268, L271, T275, L278, N282, A284, S285, R289, F292, I295, W296, L298, and Y299.

Some aspects of the present disclosure are directed to a cell comprising an rMPT disclosed herein, wherein the cell is capable of producing CBGA in the presence of GPP and OA.

Some aspects of the present disclosure are directed to a cell comprising an rMPT disclosed herein, wherein the cell is capable of producing CBGVA in the presence of GPP and DVA.

Some aspects of the present disclosure are directed to a cell comprising an rMPT disclosed herein, wherein the cell is capable of making a cannabinoid in the presence of a carbon source and, optionally, hexanoic or butyric acid.

In some embodiments, the cell expresses an exogenous membrane transporter that improves OA or DVA uptake. In some embodiments, one or more genes in the cell encoding a protein that exports OA or DVA is down-regulated or deactivated. In some embodiments, the cell expresses (e.g., overexpresses) an exogenous membrane transporter and has one or more downregulated or deactivated native exporter proteins. In some embodiments, the cell is capable of forming acetyl-CoA from a carboxylic acid. In some embodiments, the cell encodes an exogenous hexanoyl-CoA synthetase and/or butyryl-CoA synthase. In some embodiments, the cell is a yeast cell, preferably Yarrowia strain.

Some aspects of the present disclosure are directed to a method of producing CBGA or CBGVA comprising contacting a cell disclosed herein with a carbon source under suitable conditions to produce CBGA or CBGVA.

Some aspects of the present disclosure are directed to a recombinant soluble aromatic prenyltransferase (APT), the APT comprising an amino acid sequence having at least one amino acid modification as compared to a naturally occurring APT. In some embodiments, the APT comprises an amino acid sequence with at least 70% identity to APT73.74 (SEQ ID NO: 37), APT73.77 (SEQ ID NO: 38), or APT89.38 (SEQ ID NO: 39), or a functional fragment or variant thereof. In some embodiments, the APT comprises an amino acid sequence with at least one amino acid modification as compared to APT73.74 (SEQ ID NO: 37), APT73.77 (SEQ ID NO: 38), or APT89.38 (SEQ ID NO: 39), or a functional fragment thereof.

Some aspects of the present disclosure are directed to a cell comprising an APT disclosed herein, wherein the cell is capable of producing CBGA in the presence of GPP and OA.

Some aspects of the present disclosure are directed to a cell comprising an APT disclosed herein, wherein the cell is capable of producing CBGVA in the presence of GPP and DVA.

Some aspects of the present disclosure are directed to a cell comprising an APT disclosed herein, wherein the cell is capable of making a cannabinoid in the presence of a carbon source and, optionally, hexanoic or butyric acid.

In some embodiments, the cell expresses an exogenous membrane transporter that improves OA or DVA uptake. In some embodiments, one or more genes in the cell encoding a protein that exports OA or DVA is down-regulated or deactivated. In some embodiments, the cell expresses (e.g., overexpresses) an exogenous membrane transporter and has one or more downregulated or deactivated native exporter proteins. In some embodiments, the cell is capable of forming acetyl-CoA from a carboxylic acid. In some embodiments, the cell encodes an exogenous hexanoyl-CoA synthetase and/or butyryl-CoA synthase. In some embodiments, the cell is a yeast cell, preferably Yarrowia strain.

Some aspects of the present disclosure are directed to a method of producing CBGA or CBGVA comprising contacting a cell disclosed herein with a carbon source under suitable conditions to produce CBGA or CBGVA.

Some aspects of the present disclosure are directed to a fusion protein comprising a polypeptide having Geranyl diphosphate synthase activity and a polypeptide having prenyltransferase activity. In some embodiments, the fusion protein further comprises a polypeptide having polyketide cyclase (PKC) activity. In some embodiments, the polypeptide having prenyltransferase activity comprises a polypeptide sequence having at least 70% identity to the polypeptide sequence of MPT4 (SEQ ID NO: 2), MPT4.1 (SEQ ID 52), or a functional fragment thereof. In some embodiments, the polypeptide having prenyltransferase activity comprises a polypeptide sequence having at least 90% identity to SEQ ID NO: 22 (MPT21), SEQ ID NO: 23 (MPT26), or SEQ ID NO: 24 (MPT31). In some embodiments, the polypeptide having prenyltransferase activity has improved selectivity for GPP over FPP, as compared to a control membrane bound prenyltransferase. In some embodiments, the polypeptide having prenyltransferase activity has an amino acid sequence comprising portions of the amino acid sequence of SEQ ID NO: 1 (MPT1) and SEQ ID NO: 2 (MPT4) or SEQ ID NO:52 (MPT4.1). In some embodiments, the polypeptide having prenyltransferase activity comprises a polypeptide sequence having at least 70% identity to the polypeptide sequence of a soluble aromatic prenyltransferase (APT), or functional fragment thereof. In some embodiments, the APT is selected from APT73.74 (SEQ ID NO: 37), APT73.77 (SEQ ID NO: 38), and APT89.38 (SEQ ID NO: 39). In some embodiments, the polypeptide having prenyltransferase activity comprises a polypeptide sequence having at least 90% identity to APT73.74 (SEQ ID NO: 37), APT73.77 (SEQ ID NO: 38), or APT89.38 (SEQ ID NO: 39), or a functional fragment thereof. In some embodiments, the polypeptide having Geranyl diphosphate synthase activity comprises a polypeptide of SEQ ID NO: 4 (GPS1.1), SEQ ID NO: 5 (GPS2), SEQ ID NO: 6 (GPS3), or a functional fragment or functional variant thereof.

In some embodiments, the fusion protein further comprises a linker polypeptide between the polypeptide having Geranyl diphosphate synthase activity and the polypeptide having prenyltransferase activity. In some embodiments, the linker comprises a polypeptide selected from SEQ ID NO: 7-15 or 28-36.

In some embodiments, the fusion protein comprises the polypeptide sequence of GPS1.1-F11-MPT4 (SEQ ID NO: 16), GPS1.1-F5-MPT4 (SEQ ID NO: 17), GPS1.1-F9-MPT4 (SEQ ID NO: 18), GPS1.1-F10-MPT4 (SEQ ID NO: 19), GPS3-F11-MPT4 (SEQ ID NO: 20), GPS2-F11-MPT4 (SEQ ID NO: 21), GPS1.1-F5-MPT4.1 (SEQ ID NO: 54), GPS1.1-F9-MPT4.1 (SEQ ID NO: 53), GPS1.1-F10-MPT4.1 (SEQ ID NO: 55), GPS1.1-F11-MPT4.1 (SEQ ID NO: 56), GPS3-F11-MPT4.1 (SEQ ID NO: 57), GPS2-F11-MPT4.1 (SEQ ID NO: 58), GPS1.1-F16-APT73.74 (SEQ ID NO: 59), APT73.74-F17-GPS1.1 (SEQ ID NO: 60), GPS1.1-F18-APT73.74 (SEQ ID NO: 61), APT73.74-F18-GPS1.1 (SEQ ID NO: 62), GPS1.1-F16-APT73.77 (SEQ ID NO: 63), APT73.77-F17-GPS1.1 (SEQ ID NO: 64), GPS1.1-F18-APT73.77 (SEQ ID NO: 65), APT73.77-F18-GPS1.1 (SEQ ID NO: 66), GPS1.1-F16-APT89.38 (SEQ ID NO: 67), APT89.38-F17-GPS1.1 (SEQ ID NO: 68), GPS1.1-F18-APT89.38 (SEQ ID NO: 69), APT89.38-F18-GPS1.1 (SEQ ID NO: 70), or a functional fragment or variant thereof.

Some aspects of the present disclosure are directed to a fusion protein comprising a polypeptide having prenyl transferase (rMPT or APT) activity, a polypeptide having polyketide cyclase activity and a polypeptide having GPP synthase activity. In some embodiments, the polypeptide having prenyltransferase activity comprises a polypeptide sequence having at least 70% identity to the polypeptide sequence of MPT4 (SEQ ID NO: 2), or MPT4.1 (SEQ ID NO: 52), or a functional fragment thereof. In some embodiments, the polypeptide having prenyltransferase activity comprises a polypeptide sequence having at least 90% identity to SEQ ID NO: 22 (MPT21), SEQ ID NO: 23 (MPT26), or SEQ ID NO: 24 (MPT31). In some embodiments, the polypeptide having prenyltransferase activity has improved selectivity for GPP over FPP, as compared to a control membrane bound prenyltransferase In some embodiments, the polypeptide having prenyltransferase activity has an amino acid sequence comprising portions of the amino acid sequence of SEQ ID NO: 1 (MPT1) and SEQ ID NO: 2 (MPT4) or SEQ ID NO: 52 (MPT4.1).

In some embodiments, the polypeptide having prenyltransferase activity comprises a polypeptide sequence having at least 70% identity to the polypeptide sequence of a soluble aromatic prenyltransferase (APT), or functional fragment thereof. In some embodiments, the APT is selected from APT73.74 (SEQ ID NO: 37) and APT73.77 (SEQ ID NO: 38). In some embodiments, the polypeptide having prenyltransferase activity comprises a polypeptide sequence having at least 90% identity to APT73.74 (SEQ ID NO: 37) or APT73.77 (SEQ ID NO: 38), or a functional fragment thereof.

In some embodiments, the polypeptide having geranyl diphosphate synthase activity comprises a polypeptide of SEQ ID NO: 4 (GPS1.1), SEQ ID NO: 5 (GPS2), or SEQ ID NO: 6 (GPS3), or a functional fragment or functional variant thereof. In some embodiments the polyketide cyclase (PKC) can catalyze the cyclization of a tetraketide to the corresponding 5-alkyl 2,4-dihydroxy-benzoic acid. In some embodiments the polyketide cyclase produces olivetolic acid or divarinic acid. In some embodiments the polypeptide having polyketide cyclase activity comprises a polypeptide sequence having at least 70% identity to the polypeptide sequence of PKC1.0 (SEQ ID NO: 106), PKC1.1 (SEQ ID NO: 107), PKC4.33 (SEQ ID NO 108) or PKC11 (SEQ ID NO:109) or a functional fragment thereof. In other embodiments, the polypeptide having polyketide cyclase activity comprises a polypeptide sequence having at least 90% identity to SEQ ID NO: 107 (PKC1.1), SEQ ID NO: 108 (PKC4.33) or PKC11 (SEQ ID NO:109). In still further embodiments, the polypeptide having polyketide cyclase activity is PKC1.1 (SEQ ID NO: 107), PKC4.33 (SEQ ID NO: 108) or PKC11 (SEQ ID NO:109).

In some embodiments, at least two of the polypeptides in the triple fusion protein having prenyltransferase activity, GPP synthase activity or polyketide cyclase activity are fused together with a linker comprising 1 to 40 amino acids. In another embodiment, at least two of the polypeptides in the triple fusion protein having prenyltransferase activity, GPP synthase activity or polyketide cyclase activity are fused together without a linker. In still another embodiment, two polypeptides of the triple fusion protein having prenyltransferase activity, GPP synthase activity or polyketide cyclase activity are fused without a linker while two polypeptides of the triple fusion protein having prenyltransferase activity, GPP synthase activity or polyketide activity are fused with a linker comprising 1-40 amino acids. In some embodiments, the order in which the three polypeptides having prenyltransferase activity, GPP synthase activity and polyketide cyclase activity are fused is not particularly limited and includes all mathematically possible combinations without repetitions. In a preferred embodiment, the three polypeptides having prenyltransferase activity, GPP synthase activity and polyketide cyclase activity are fused in the order: GPP synthase fused to N-terminus of PK cyclase fused to N-terminus of prenyl transferase (e.g., GPS-PKC-MPT or GPS-PKC-APT). In other embodiments, the order of the fused proteins can be different when the polypeptide having prenyltransferase is soluble aromatic prenyl tranferase. For example, the order of the fused proteins can be GPS-APT-PKC, APT-PKC-GPS or APT-GPS-PKC. In still other embodiments, the order of the fused proteins of the triple fused protein is selected from the group consisting of GPS-MPT-PKC, MPT-GPS-PKC, MPT-PKC-GPS, PKC-GPS-APT, PKC-GPS-MPT, PKC-APT-GPS and PKC-MPT-GPS. In some embodiments, the triple fusion protein comprises the polypeptide sequence of GPS1.1-PKC4.33-APT73.77 (SEQ ID NO: 112), GPS1.1-F11-PKC4.33-F18-APT73.77 (SEQ ID NO: 111), GPS1.1-F11-PKC1.1-F18-APT73.77 (SEQ ID NO: 110), PKC4.33-GPS1.1-F11-MPT21.3 (SEQ ID NO: 104), PKC4.33-F11-GPS1.1-F11-MPT21.3 (SEQ ID NO: 103), PKC1.1-F11-GPS1.1-F11-MPT21.3 (SEQ ID NO: 102), PKC4.33-GPS1.1-F11-MPT4.1 (SEQ ID NO: 101), PKC4.33-F11-GPS1.1-F11-MPT4.1 (SEQ ID NO: 100), PKC1.1-F11-GPS1.1-F11-MPT4.1 (SEQ ID NO: 99), GPS1.1-PKC4.33-MPT21.3 (SEQ ID NO: 98), GPS1.1-PKC4.33-MPT4.1 (SEQ ID NO: 97), GPS1.1-F11-PKC4.33-F11-MPT21.3 (SEQ ID NO: 96), GPS1.1-F11-PKC4.33-F11-MPT4.1 (SEQ ID NO: 95), GPS1.1-F11-PKC1.1-F11-MPT21.3 (SEQ ID NO: 94), GPS1.1-F11-PKC1.1-F11-MPT4.1 (SEQ ID NO: 93), or GPS1.1-F11-PKC1.1-F11-MPT4 (SEQ ID NO: 92), GPS1.1-F11-PKC11-F11-MPT4 (SEQ ID NO: 113) or GPS1.1-F11-PKC11-F11-MPT4.1 (SEQ ID NO: 114).

Some aspects of the present disclosure are directed to a cell comprising a fusion protein described herein, wherein the cell is capable of producing CBGA in the presence of OA and GPP.

Some aspects of the present disclosure are directed to a cell comprising a fusion protein described herein, wherein the cell is capable of producing CBGVA in the presence of DVA and a GPP.

Some aspects of the present disclosure are directed to a cell comprising a fusion protein described herein, wherein the cell is capable of making a cannabinoid in the presence of a carbon source and, optionally, hexanoic or butyric acid.

In some embodiments, the cell expresses an exogenous membrane transporter that improves OA or DVA uptake. In some embodiments, one or more genes in the cell encoding a protein that exports OA or DVA is down-regulated or deactivated In some embodiments, the cell expresses (e.g., overexpresses) an exogenous membrane transporter and has one or more downregulated or deactivated native exporter proteins. In some embodiments, the cell is capable of forming acetyl-CoA from a carboxylic acid. In some embodiments, the cell encodes an exogenous hexanoyl-CoA and/or butyryl-CoA synthetase. In some embodiments, the cell is a yeast cell, preferably Yarrowia strain.

Some aspects of the present disclosure are directed to a method of producing CBGA or CBGVA comprising contacting a cell described herein with a carbon source under suitable conditions to produce CBGA or CBGVA.

### BRIEF DESCRIPTION OF THE DRAWINGS

The patent or application file contains at least one drawing executed in color. Copies of this patent or patent application publication with color drawings will be provided by the Office upon request and payment of the necessary fee.

FIG. 1 shows CBGA derivatives synthesized by CBGA synthase(s) described herein and provides a list of cannabinoids that can be synthesized using CBGA synthase(s) described herein and in combination with a CBDA, CBCA, THCA, or other synthase.

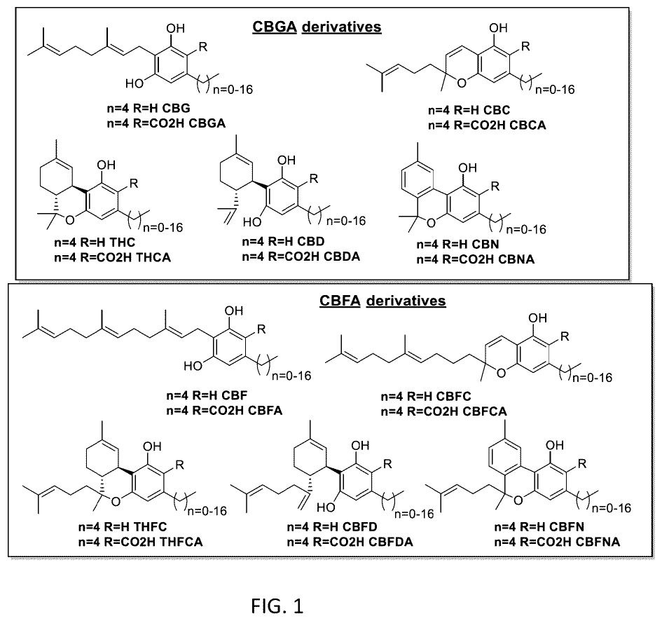

FIG. 2 shows a structural alignment of MPT1 (gray) and MPT4 (brown) that was used to guide the recombination and chimera design. The area between the ball filled flat surface is the membrane lipid layer.

### DETAILED DESCRIPTION OF THE INVENTION

Some aspects of the present disclosure are directed to a recombinant membrane-bound prenyltransferase (rMPT), the rMPT comprising an amino acid sequence having at least one amino acid modification as compared to a naturally occurring membrane-bound prenyltransferase.

Amino acid modifications may be amino acid substitutions, amino acid deletions and/or amino acid insertions. Amino acid substitutions may be conservative amino acid substitutions or non-conservative amino acid substitutions. A conservative replacement (also called a conservative mutation, a conservative substitution or a conservative variation) is an amino acid replacement in a protein that changes a given amino acid to a different amino acid with similar biochemical properties (e.g. charge, hydrophobicity and size). As used herein, “conservative variations” refer to the replacement of an amino acid residue by another, biologically similar residue. Examples of conservative variations include the substitution of one hydrophobic residue such as isoleucine, valine, leucine or methionine for another; or the substitution of one polar residue for another, such as the substitution of arginine for lysine, glutamic for aspartic acids, or glutamine for asparagine, and the like. Other illustrative examples of conservative substitutions include the changes of: alanine to serine; arginine to lysine; asparagine to glutamine or histidine; aspartate to glutamate; cysteine to serine; glutamine to asparagine; glutamate to aspartate; glycine to proline; histidine to asparagine or glutamine; isoleucine to leucine or valine; leucine to valine or isoleucine; lysine to arginine, glutamine, or glutamate; methionine to leucine or isoleucine; phenylalanine to tyrosine, leucine or methionine; serine to threonine; threonine to serine; tryptophan to tyrosine; tyrosine to tryptophan or phenylalanine; valine to isoleucine or leucine, and the like.

In some embodiments, the rMPT comprises an amino acid sequence with at least 70% identity to SEQ ID NO: 1 (MPT1) or SEQ ID NO: 2 (MPT4), wherein the rMPT comprises at least one amino acid modification as compared to SEQ ID NO: 1 or 2. In some embodiments, the rMPT comprises an amino acid sequence with at least 75%, 80%, 85%, 90%, 95%, 99%, 99.5%, or 99.9% identity to SEQ ID NO: 1 or 2.

In some embodiments, the rMPT comprises an amino acid sequence comprising portions of the amino acid sequence of SEQ ID NO: 1 (MPT1) and SEQ ID NO: 2 (MPT4), wherein the amino acid sequence comprises at least one amino acid modification as compared to SEQ ID NO: 1 or 2. In some embodiments, a portion comprises a contiguous amino acid sequence comprising at least 2, 3, 4, 5, 6, 7, 8, 9, 10, 11, 12, 13, 14, 15, 20, 25, 30, 35, 40, or 45 amino acids. In some embodiments, the rMPT comprises at least 2, 3, 4, 5, 6, 7, 8, 9, 10, or more portions from SEQ ID NO: 1 (MPT1) and/or SEQ ID NO: 2 (MPT4).

In some embodiments, the rMPT comprises a functional fragment of MPT4 (SEQ ID NO: 2). In some embodiments, the functional fragment of MPT4 (SEQ ID NO: 2) has at least the first 1, 2, 3, 4, 5, 6, 7, 8, 9, 10, 11, 12, 13, 14, 15, 16, 17, 18, 19, or 20 amino acids deleted from the amino terminus. In some embodiments, the functional fragment of MPT4 (SEQ ID NO: 2) has at least the first 1, 2, 3, 4, 5, 6, 7, 8, 9, 10, 11, 12, 13, 14, 15, 16, 17, 18, 19, or 20 amino acids deleted from the carboxy terminus.

In some embodiments, the rMPT comprises an amino acid sequence of SEQ ID NO: 2 (MPT4) with one or more of the following amino acid positions comprising a mutation (insertion, deletion, substitution): R22, P23, Y24, V25, V26, K27, G28, M29, S31, A33, F66, N67, A70, A71, D80, I81, I84, N85, K86, P87, D88, L89, L91, V92, Y139, S140, F154, L155, I158, S159, S160, V162, T194, V195, G197, M198, I200, A201, F202, A203, K204, D208, I209, G211, D212, L267, A268, L271, T275, L278, N282, A284, S285, R289, F292, I295, W296, L298, Y299. In some embodiments, the rMPT comprises at least two, three, four, five, six, seven, eight, nine, or ten of the mutations. In some embodiments, the rMPT further comprises a truncation (e.g., 1-10 amino acids) at the C and/or N terminus.

In some embodiments, the rMPT comprises an amino acid sequence with at least 70%, 75%, 80%, 85%, 90%, 95%, 99%, 99.5%, 99.9%, or 100% identity to SEQ ID NO: 22 (MPT21). In some embodiments, the rMPT comprises an amino acid sequence with at least 90% identity to SEQ ID NO: 22 (MPT21). In some embodiments, the rMPT comprises a functional fragment of SEQ ID NO: 22 (MPT21) or an amino acid sequence with at least 70%, 75%, 80%, 85%, 90%, 95%, 99%, 99.5%, 99.9%, or 100% identity to SEQ ID NO: 22 (MPT21). In some embodiments, the functional fragment of SEQ ID NO: 22 (MPT21) has at least the first 1, 2, 3, 4, 5, 6, 7, 8, 9, 10, 11, 12, 13, 14, 15, 16, 17, 18, 19, or 20 amino acids deleted from the amino terminus. In some embodiments, the functional fragment of SEQ ID NO: 22 (MPT21) has at least the first 1, 2, 3, 4, 5, 6, 7, 8, 9, 10, 11, 12, 13, 14, 15, 16, 17, 18, 19, or 20 amino acids deleted from the carboxy terminus.

In some embodiments, the rMPT comprises an amino acid sequence of SEQ ID NO: 22 (MPT21) with one or more of the following amino acid positions comprising a mutation (insertion, deletion, substitution): R22, P23, Y24, V25, V26, K27, G28, M29, S31, A33, F66, N67, A70, A71, I81, I84, N85, K86, P87, D88, L89, L91, Y139, S140, F154, L155, I158, S159, S160, V162, T194, V195, G197, M198, I200, A201, F202, A203, K204, D208, I209, G211, D212, L267, A268, L271, T275, FL278, N282, A284, S285, R289, F292, I295, W296, L298, Y299. In some embodiments, the rMPT comprises at least two, three, four, five, six, seven, eight, nine, or ten of the mutations. In some embodiments, the rMPT further comprises a truncation (e.g., 1-10 amino acids) at the C and/or N terminus.

In some embodiments, the rMPT comprises an amino acid sequence with at least 70%, 75%, 80%, 85%, 90%, 95%, 99%, 99.5%, or 99.9%, or 100% identity to SEQ ID NO: 23 (MPT26). In some embodiments, the rMPT comprises an amino acid sequence with at least 90% identity to SEQ ID NO: 23 (MPT26). In some embodiments, the rMPT comprises a functional fragment of SEQ ID NO: 23 (MPT26) or an amino acid sequence with at least 70%, 75%, 80%, 85%, 90%, 95%, 99%, 99.5%, 99.9%, or 100% identity to SEQ ID NO: 23 (MPT26). In some embodiments, the functional fragment of SEQ ID NO: 23 (MPT26) has at least the first 1, 2, 3, 4, 5, 6, 7, 8, 9, 10, 11, 12, 13, 14, 15, 16, 17, 18, 19, or 20 amino acids deleted from the amino terminus. In some embodiments, the functional fragment of SEQ ID NO: 23 (MPT26) has at least the first 1, 2, 3, 4, 5, 6, 7, 8, 9, 10, 11, 12, 13, 14, 15, 16, 17, 18, 19, or 20 amino acids deleted from the carboxy terminus.

In some embodiments, the rMPT comprises an amino acid sequence of SEQ ID NO: 23 (MPT26) with one or more of the following amino acid positions comprising a mutation (insertion, deletion, substitution): R22, P23, Y24, V25, V26, K27, G28, M29, S31, A33, F66, N67, A70, A71, I81, I84, N85, K86, P87, D88, L89, L91, Y139, S140, F154, L155, I158, S159, S160, V162, T194, V195, G197, M198, I200, A201, F202, A203, K204, D208, I209, G211, D212, L267, A268, L271, T275, FL278, N282, A284D284, S285P285, R289, F292, 1295, W296, L298, Y299. In some embodiments, the rMPT comprises at least two, three, four, five, six, seven, eight, nine, or ten of the mutations. In some embodiments, the rMPT further comprises a truncation (e.g., 1-10 amino acids) at the C and/or N terminus.

In some embodiments, the rMPT comprises an amino acid sequence with at least 70%, 75%, 80%, 85%, 90%, 95%, 99%, 99.5%, or 99.9%, or 100% identity to SEQ ID NO: 24 (MPT31). In some embodiments, the rMPT comprises an amino acid sequence with at least 90% identity to SEQ ID NO: 24 (MPT31). In some embodiments, the rMPT comprises a functional fragment of SEQ ID NO: 24 (MPT31) or an amino acid sequence with at least 70%, 75%, 80%, 85%, 90%, 95%, 99%, 99.5%, 99.9%, or 100% identity to SEQ ID NO: 24 (MPT31). In some embodiments, the functional fragment of SEQ ID NO: 24 (MPT31) has at least the first 1, 2, 3, 4, 5, 6, 7, 8, 9, 10, 11, 12, 13, 14, 15, 16, 17, 18, 19, or 20 amino acids deleted from the amino terminus. In some embodiments, the functional fragment of SEQ ID NO: 24 (MPT31) has at least the first 1, 2, 3, 4, 5, 6, 7, 8, 9, 10, 11, 12, 13, 14, 15, 16, 17, 18, 19, or 20 amino acids deleted from the carboxy terminus.

In some embodiments, the rMPT comprises an amino acid sequence of SEQ ID NO: 24 (MPT31) with one or more of the following amino acid positions comprising a mutation (insertion, deletion, substitution): R22, P23, Y24, V25, V26, K27, G28, M29, S31, A33, F66, N67, A70, A71, D80 (H80-MPT31), I81, I84, N85, K86, P87, D88, L89, L91, V92 (A92-MPT31), Y139, S140, F154, L155, I158, S159, S160, V162, T194, V195, G197, M198, I200, A201, F202, A203, K204, D208, I209, G211, D212, L267, A268, L271, T275, FL278, N282, A284D284, S285P285, R289, F292, I295, W296, L298, Y299. In some embodiments, the rMPT comprises at least two, three, four, five, six, seven, eight, nine, or ten of the mutations. In some embodiments, the rMPT further comprises a truncation (e.g., 1-10 amino acids) at the C and/or N terminus.

Some aspects of the present disclosure are directed to a recombinant soluble aromatic prenyltransferase (APT), the APT comprising an amino acid sequence having at least one amino acid modification as compared to APT73.74 (SEQ ID NO: 37), APT73.77 (SEQ ID NO: 38) and APT89.38 (SEQ ID NO: 39). In some embodiments, the APT comprises an amino acid sequence with at least 70%, 75%, 80%, 85%, 90%, 95%, 99%, 99.5%, or 99.9%, or 100% identity to SEQ ID NO: 37 (APT73.74). In some embodiments, the APT comprises an amino acid sequence with at least 90% identity to SEQ ID NO: 37 (APT73.74). In some embodiments, the APT comprises a functional fragment of SEQ ID NO: 37 (APT73.74) or an amino acid sequence with at least 70%, 75%, 80%, 85%, 90%, 95%, 99%, 99.5%, 99.9%, or 100% identity to SEQ ID NO: 37 (APT73.74). In some embodiments, the functional fragment of SEQ ID NO: 37 (APT73.74) has at least 1, 2, 3, 4, 5, 6, 7, 8, 9, 10, 11, 12, 13, 14, 15, 16, 17, 18, 19, or 20 amino acids added to the carboxy terminus.

In some embodiments, the APT comprises an amino acid sequence with at least 70%, 75%, 80%, 85%, 90%, 95%, 99%, 99.5%, or 99.9%, or 100% identity to SEQ ID NO: 38 (APT73.77). In some embodiments, the APT comprises an amino acid sequence with at least 90% identity to SEQ ID NO: 38 (APT73.77). In some embodiments, the APT comprises a functional fragment of SEQ ID NO: 38 (APT73.77) or an amino acid sequence with at least 70%, 75%, 80%, 85%, 90%, 95%, 99%, 99.5%, 99.9%, or 100% identity to SEQ ID NO: 38 (APT73.77). In some embodiments, the functional fragment of SEQ ID NO: 38 (APT73.77) has at least 1, 2, 3, 4, 5, 6, 7, 8, 9, 10, 11, 12, 13, 14, 15, 16, 17, 18, 19, or 20 amino acids added to the carboxy terminus.

In some embodiments, the APT comprises an amino acid sequence with at least 70%, 75%, 80%, 85%, 90%, 95%, 99%, 99.5%, or 99.9%, or 100% identity to SEQ ID NO: 39 (APT89.38). In some embodiments, the APT comprises an amino acid sequence with at least 90% identity to SEQ ID NO: 39 (APT89.38). In some embodiments, the APT comprises a functional fragment of SEQ ID NO: 39 (APT89.38) or an amino acid sequence with at least 70%, 75%, 80%, 85%, 90%, 95%, 99%, 99.5%, 99.9%, or 100% identity to SEQ ID NO: 39 (APT89.38). In some embodiments, the functional fragment of SEQ ID NO: 39 (APT89.38) has at least 1, 2, 3, 4, 5, 6, 7, 8, 9, 10, 11, 12, 13, 14, 15, 16, 17, 18, 19, or 20 amino acids added to the carboxy terminus.

“Identity” refers to the extent to which the sequence of two or more nucleic acids or polypeptides is the same. In some embodiments, percent identity between a sequence of interest and a second sequence over a window of evaluation, e.g., over the length of the sequence of interest, may be computed by aligning the sequences, determining the number of residues (nucleotides or amino acids) within the window of evaluation that are opposite an identical residue allowing the introduction of gaps to maximize identity, dividing by the total number of residues of the sequence of interest or the second sequence (whichever is greater) that fall within the window, and multiplying by 100. When computing the number of identical residues needed to achieve a particular percent identity, fractions are to be rounded to the nearest whole number. Percent identity can be calculated with the use of a variety of computer programs known in the art. For example, computer programs such as BLAST2, BLASTN, BLASTP, Gapped BLAST, etc., generate alignments and provide percent identity between sequences of interest. The algorithm of Karlin and Altschul (Karlin and Altschul, Proc. Natl. Acad. Sci. USA 87:22264-2268, 1990) modified as in Karlin and Altschul, Proc. Natl. Acad. Sci. USA 90:5873-5877, 1993 is incorporated into the NBLAST and XBLAST programs of Altschul et al. (Altschul, et al., J. Mol. Biol. 215:403-410, 1990). To obtain gapped alignments for comparison purposes, Gapped BLAST is utilized as described in Altschul et al. (Altschul, et al. Nucleic Acids Res. 25: 3389-3402, 1997). When utilizing BLAST and Gapped BLAST programs, the default parameters of the respective programs may be used. A PAM250 or BLOSUM62 matrix may be used. Software for performing BLAST analyses is publicly available through the National Center for Biotechnology Information (NCBI). See the Web site having URL ncbi.nlm.nih.gov for these programs. In a specific embodiment, percent identity is calculated using BLAST2 with default parameters as provided by the NCBI.

In some embodiments, the rMPT comprises a fusion domain. In some embodiments, the fusion domain improves expression and/or the overall activity of the enzyme.

In some embodiments, the fusion domain targets the protein to a specific compartment of the cell such as the ER, vacuole, Golgi, peroxisome, lipid body (e.g., oleosome), or targets secretion of the protein from the cell into the outer membrane. In certain embodiments, the rMPT may contain one or more modifications that are capable of stabilizing the rMPT.

In some embodiments, the rMPT is capable of converting olivetolic acid (OA) and geranyl diphosphate (GPP) to one or more products comprising cannabigerolic acid (CBGA). In some embodiments, the rMPT is capable of producing CBGA in a cell free system, in a yeast cell, in a bacterial cell, in an algae cell, or in a plant cell. In some embodiments, the one or more products comprise at least 10%, 15%, 20%, 25%, 30%, 35%, 40%, 45%, 50%, 55%, 60%, 65%, 70%, 75%, 80%, 85%, 90%, 95%, or substantially 100% CBGA. In some embodiments, at least about 50% of the one or more products is CBGA. In some embodiments, more than about 90% of the one or more products is CBGA. In some embodiments, the rMPT has a rate of formation of cannabigerolic acid (CBGA) from olivetolic acid (OA) and geranyl diphosphate (GPP) that is greater than the rate of formation of CBGA from OA and GPP by MPT4 under the same conditions. In some embodiments, the rate of formation of CBGA from OA and GPP is at least 1.1-fold, 1.2-fold, 1.3-fold, 1.4-fold, 1.5-fold, 1.6-fold, 1.7-fold, 1.8-fold, 1.9-fold, 2-fold, 2.5-fold, 5-fold, 10-fold, or more as compared to the rate of formation of CBGA from OA and GPP by MPT4 under the same conditions.

In some embodiments, the rMPT is capable of converting olivetolic acid (OA) and farnesyl pyrophosphate (FPP) to one or more cannabinoids, cannabinoid derivatives or cannabinoid analogues. In some embodiments, the rMPT is capable of producing cannabinoids, cannabinoid derivatives or cannabinoid analogues in a cell free system, in a yeast cell, in a bacterial cell, in an algae cell, or in a plant cell. In some embodiments, the activity of the rMPT for converting OA and FPP to one or more cannabinoids, cannabinoid derivatives or cannabinoid analogues is at least about 10%, 15%, 20%, 25%, 30%, 35%, 40%, 45%, 50%, 55%, 60%, 65%, 70%, 75%, 80%, 85%, 90%, 95%, or substantially 100% of the activity of the rMPT for converting OA and GPP to one or more cannabinoids, cannabinoid derivatives or cannabinoid analogues.

In some embodiments, the rMPT produces CBGA and FCBGA from olivetolic acid (OA) and geranyl diphosphate (GPP) and farnesyl diphosphate (FPP) respectively, at a CBGA/FCBGA ratio that is greater than the ratio of CBGA/FCBGA formation ratio from OA and GPP and FPP by MPT4 under the same conditions . As used herein and in some embodiments, “greater than” is at least 1.1-fold, 1.2-fold, 1.3-fold, 1.4-fold, 1.5-fold, 1.6-fold, 1.7-fold, 1.8-fold, 1.9-fold, 2-fold, 2.5-fold, 5-fold, 10-fold, or more as compared to the relevant control.

Recombinant rMPT with reduced or no FCBGA formation activity can be advantageous in some situations. In some embodiments, the rMPT has a rate of formation of cannabigerovarinic acid (CBGVA) from divarinic acid (DVA) and geranyl diphosphate (GPP) that is greater than the rate of formation of CBGVA from DVA and GPP by MPT4 under the same conditions. In some embodiments, the rMPT has a ratio of CBGVA to F-CBGVA formation from DVA and GPP and FPP that is greater than the ratio of CBGVA to F-CBGVA from DVA and GPP and FPP by MPT4 under the same conditions. As used herein and in some embodiments, “greater than” is at least 1.1-fold, 1.2-fold, 1.3-fold, 1.4-fold, 1.5-fold, 1.6-fold, 1.7-fold, 1.8-fold, 1.9-fold, 2-fold, 2.5-fold, 5-fold, 10-fold, or more as compared to the relevant control. In some embodiments, the rMPT does not form F-CBGVA.

In some embodiments, the rMPT has a rate of formation of CBGA from OA and GPP that is at least 1.2-fold greater (e.g., 1.2-fold, 1.3-fold, 1.4-fold, 1.5-fold, 1.6-fold, 1.7-fold, 1.8-fold, 1.9-fold, 2-fold, 2.5-fold, 5-fold, 10-fold, or more) than the rate of formation of CBGA from OA and GPP by MPT4 under the same conditions.

Cannabinoids, cannabinoid derivatives and cannabinoid analogues as recited herein are not limited. In some embodiments, cannabinoids may include, but are not limited to, cannabichromene (CBC) type (e.g. cannabichromenic acid), cannabigerol (CBG) type (e.g. cannabigerolic acid), cannabidiol (CBD) type (e.g. cannabidiolic acid), Δ⁹-trans-tetrahydrocannabinol (Δ⁹-THC) type (e.g. Δ⁹-tetrahydrocannabinolic acid), Δ⁸-trans-tetrahydrocannabinol (Δ⁸-THC) type, cannabicyclol (CBL) type, cannabielsoin (CBE) type, cannabinol (CBN) type, cannabinodiol (CBND) type, cannabitriol (CBT) type, cannabigerolic acid (CBGA), cannabigerolic acid monomethylether (CBGAM), cannabigerol (CBG), cannabigerol monomethylether (CBGM), cannabigerovarinic acid (CBGVA), cannabigerovarin (CBGV), cannabichromenic acid (CBCA), cannabichromene (CBC), cannabichromevarinic acid (CBCVA), cannabichromevarin (CBCV), cannabidiolic acid (CBDA), cannabidiol (CBD), cannabidiol monomethylether (CBDM), cannabidiol-C₄ (CBD-C₄), cannabidivarinic acid (CBDVA), cannabidivarin (CBDV), cannabidiorcol (CBD-C₁), Δ⁹-tetrahydrocannabinolic acid A (THCA-A), Δ⁹-tetrahydrocannabinolic acid B (THCA-B), Δ⁹-tetrahydrocannabinol (THC), Δ⁹-tetrahydrocannabinolic acid-C₄ (THCA-C₄), Δ⁹-tetrahydrocannabinol-C₄ (THC-C₄), Δ⁹-tetrahydrocannabivarinic acid (THCVA), Δ⁹-tetrahydrocannabivarin (THCV), Δ⁹-tetrahydrocannabiorcolic acid (THCA-C₁), Δ⁹-tetrahydrocannabiorcol (THC-C₁), Δ⁷-cis-iso-tetrahydrocannabivarin, Δ⁸-tetrahydrocannabinolic acid (⁸-THCA), Δ⁸-tetrahydrocannabinol (Δ⁸-THC), cannabicyclolic acid (CBLA), cannabicyclol (CBL), cannabicyclovarin (CBLV), cannabielsoic acid A (CBEA-A), cannabielsoic acid B (CBEA-B), cannabielsoin (CBE), cannabielsoinic acid, cannabicitranic acid, cannabinolic acid (CBNA), cannabinol (CBN), cannabinol methylether (CBNM), cannabinol-C₄, (CBN-C₄), cannabivarin (CBV), cannabinol-C₂ (CNB-C₂), cannabiorcol (CBN-C₁), cannabinodiol (CBND), cannabinodivarin (CBVD), cannabitriol (CBT), 10-ethyoxy-9-hydroxy-delta-6a-tetrahydrocannabinol, 8,9-dihydroxyl-delta-6a-tetrahydrocannabinol, cannabitriolvarin (CBTVE), dehydrocannabifuran (DCBF), cannabifuran (CBF), cannabichromanon (CBCN), cannabicitran (CBT), 10-oxo-delta-6a-tetrahydrocannabinol (OTHC), delta-9-cis-tetrahydrocannabinol (cis-THC), 3,4,5,6-tetrahydro-7-hydroxy-alpha-alpha-2-trimethyl-9-n-propyl-2,6-methano-2H-1-benzoxocin-5-methanol (OH-iso-HHCV), cannabiripsol (CBR), and trihydroxy-delta-9-tetrahydrocannabinol (triOH-THC).

In some embodiments, the rMPT is capable of converting divarinic acid (DVA) and GPP to one or more cannabinoids, cannabinoid derivatives or cannabinoid analogues. The cannabinoids are not limited and may be any disclosed herein. In some embodiments, the rMPT is capable of producing cannabinoids, cannabinoid derivatives or cannabinoid analogues in a cell free system, in a yeast cell, in a bacterial cell, in an algae cell, or in a plant cell. In some embodiments, the activity of the rMPT for converting DVA and FPP to one or more cannabinoids, cannabinoid derivatives or cannabinoid analogues is at least about 10%, 15%, 20%, 25%, 30%, 35%, 40%, 45%, 50%, 55%, 60%, 65%, 70%, 75%, 80%, 85%, 90%, 95%, or substantially 100% of the activity of the rMPT for converting OA and GPP to one or more cannabinoids, cannabinoid derivatives or cannabinoid analogues.

### Fusion Proteins

Some aspects of the present disclosure are directed to a fusion protein comprising a polypeptide having geranyl diphosphate (GPP) synthase activity and a polypeptide having prenyltransferase activity. As used herein, “GPP synthase activity” is the ability to catalyze the condensation of dimethylallyl diphosphate and isopentenyl diphosphate to geranyl diphosphate. As used herein, “ prenyltransferase activity” is the ability to catalyze the transfer of a prenyl group from one compound (donor) to another (acceptor).

In some embodiments, the polypeptide having prenyltransferase activity comprises a polypeptide sequence having at least 70% identity to the polypeptide sequence of MPT4 (SEQ ID NO: 2), or a functional fragment or variant thereof. In some embodiments, the polypeptide having prenyltransferase activity comprises a polypeptide sequence having at least 75%, 80%, 85%, 90%, 95%, 99%, 99.5%, or 99.9% identity to SEQ ID NO: 2.

In some embodiments, the polypeptide having prenyltransferase activity comprises a polypeptide sequence having at least 70% identity to the polypeptide sequence of MPT4.1 (SEQ ID NO: 52), or a functional fragment or variant thereof. In some embodiments, the polypeptide having prenyltransferase activity comprises a polypeptide sequence having at least 75%, 80%, 85%, 90%, 95%, 99%, 99.5%, or 99.9% identity to SEQ ID NO: 52. In some embodiments, the polypeptide having prenyltransferase activity comprises a functional fragment of MPT4.1 (SEQ ID NO: 52).

In some embodiments, the polypeptide having prenyltransferase activity comprises a polypeptide sequence having at least 75%, 80%, 85%, 90%, 95%, 99%, 99.5%, or 99.9% identity to SEQ ID NO: 22 (MPT21) or a functional fragment thereof. In some embodiments, the polypeptide having prenyltransferase activity comprises a polypeptide sequence having at least 90% identity to SEQ ID NO: 22 (MPT21) or a functional fragment thereof. In some embodiments, the polypeptide having prenyltransferase activity comprises a polypeptide sequence having at least 75%, 80%, 85%, 90%, 95%, 99%, 99.5%, or 99.9% identity to SEQ ID NO: 23 (MPT26) or a functional fragment thereof. In some embodiments, the polypeptide having prenyltransferase activity comprises a polypeptide sequence having at least 90% identity to SEQ ID NO: 23 (MPT26) or a functional fragment thereof. In some embodiments, the polypeptide having prenyltransferase activity comprises a polypeptide sequence having at least 75%, 80%, 85%, 90%, 95%, 99%, 99.5%, or 99.9% identity to SEQ ID NO: 24 (MPT31) or a functional fragment thereof. In some embodiments, the polypeptide having prenyltransferase activity comprises a polypeptide sequence having at least 90% identity to SEQ ID NO: 24 (MPT31) or a functional fragment thereof.

In some embodiments, the polypeptide having prenyltransferase activity has improved selectivity for GPP over FPP, as compared to the same unfused prenyltransferase. In some embodiments, the polypeptide having prenyltransferase activity has at least 1.1-fold, 1.2-fold, 1.3-fold, 1.4-fold, 1.5-fold, 1.6-fold, 1.7-fold, 1.8-fold, 1.9-fold, 2-fold, 2.5-fold, 5-fold, 10-fold, or higher selectivity for GPP over FPP as compared to the same unfused prenyltransferase. In some embodiments the polypeptide produces a CBGA and FCBGA in higher CBGA/FCBGA ratio compared to the same unfused prenyltransferase.

In some embodiments, the polypeptide having prenyltransferase activity has an amino acid sequence comprising portions of the amino acid sequence of SEQ ID NO: 1 (MPT1) and SEQ ID NO: 2 (MPT4). In some embodiments, a portion comprises a contiguous amino acid sequence comprising at least 3, 4, 5, 6, 7, 8, 9, 10, 11, 12, 13, 14, 15, 20, 25, 30, 35, 40, or 45 amino acids. In some embodiments, the polypeptide having prenyltransferase activity comprises 2, 3, 4, 5, 6, 7, 8, 9, 10, or more portions of the amino acid sequence of SEQ ID NO: 1 (MPT1) and/or SEQ ID NO: 2 (MPT4).

In some embodiments, the polypeptide having prenyltransferase activity comprises a polypeptide sequence having at least 70% identity to the polypeptide sequence of a soluble aromatic prenyltransferase (APT), or functional fragment thereof. In some embodiments, the polypeptide having prenyltransferase activity comprises a polypeptide sequence having at least 75%, 80%, 85%, 90%, 95%, 99%, 99.5%, or 99.9% identity to the polypeptide sequence of a soluble prenyltransferase (APT), or functional fragment thereof. In some embodiments, the APT is selected from APT73.74 (SEQ ID NO: 37), APT74.77 (SEQ ID NO: 38) and APT89.38 (SEQ ID NO: 39).

In some embodiments, the polypeptide having prenyltransferase activity comprises a polypeptide sequence having at least 75%, 80%, 85%, 90%, 95%, 99%, 99.5%, or 99.9% identity to SEQ ID NO: 37 (APT73.74) or a functional fragment thereof. In some embodiments, the polypeptide having prenyltransferase activity comprises a polypeptide sequence having at least 90% identity to SEQ ID NO: 37 (APT73.74) or a functional fragment thereof.

In some embodiments, the polypeptide having prenyltransferase activity comprises a polypeptide sequence having at least 75%, 80%, 85%, 90%, 95%, 99%, 99.5%, or 99.9% identity to SEQ ID NO: 38 (APT73.77) or a functional fragment thereof. In some embodiments, the polypeptide having prenyltransferase activity comprises a polypeptide sequence having at least 90% identity to SEQ ID NO: 38 (APT73.77) or a functional fragment thereof.

In some embodiments, the polypeptide having prenyltransferase activity comprises a polypeptide sequence having at least 75%, 80%, 85%, 90%, 95%, 99%, 99.5%, or 99.9% identity to SEQ ID NO: 39 (APT89.38) or a functional fragment thereof. In some embodiments, the polypeptide having prenyltransferase activity comprises a polypeptide sequence having at least 90% identity to SEQ ID NO: 39 (APT89.38) or a functional fragment thereof.

In some embodiments, the polypeptide having Geranyl diphosphate synthase activity comprises a polypeptide of SEQ ID NO: 4 (GPS1.1), SEQ ID NO: 5 (GPS2), SEQ ID NO: 6 (GPS3), or a functional fragment or variant thereof. In some embodiments, the polypeptide having Geranyl diphosphate synthase activity comprises a polypeptide sequence with at least 75%, 80%, 85%, 90%, 95%, 99%, 99.5%, or 99.9% identity to SEQ ID NO: 4 (GPS1.1). In some embodiments, the polypeptide having Geranyl diphosphate synthase activity comprises a polypeptide sequence with at least 75%, 80%, 85%, 90%, 95%, 99%, 99.5%, or 99.9% identity to SEQ ID NO: 5 (GPS2). In some embodiments, the polypeptide having Geranyl diphosphate synthase activity comprises a polypeptide sequence with at least 75%, 80%, 85%, 90%, 95%, 99%, 99.5%, or 99.9% identity to SEQ ID NO: 6 (GPS3).

In some embodiments, the fusion protein further comprises a linker polypeptide between the polypeptide having Geranyl diphosphate synthase activity (GPS) and the polypeptide having prenyltransferase activity. The linker is not limited and may be any suitable linker. For example, a linker can be a short polypeptide (e.g., 15-52 amino acids). Often a linker is composed of small amino acid residues such as serine, glycine, and/or alanine. A heterologous domain could comprise a transmembrane domain, a secretion signal domain, etc. In some embodiments, the linker is a polypeptide. In some embodiments, the polypeptide is 5 to 52 amino acids in length. In some embodiments, the linker comprises a polypeptide selected from SEQ ID NO: 7, 8, 9, 10, 11, 12, 13, 14, 15, 28, 29, 30, 31, 32, 33, 34, 35, or 36.

In some embodiments the geranyl diphosphate is fused to the N-terminus of the prenyl transferase. In other embodiments the geranyl diphosphate is fused to the C-terminus of the prenyl transferase. In some embodiments, the fusion protein comprises the polypeptide sequence of GPS1.1-F11-MPT4 (SEQ ID NO: 16) or a polypeptide sequence having at least 75%, 80%, 85%, 90%, 95%, 99%, 99.5%, or 99.9% identity to SEQ ID NO: 16. In some embodiments, the fusion protein comprises the polypeptide sequence of GPS1.1-F5-MPT4 (SEQ ID NO: 17) or a polypeptide sequence having at least 75%, 80%, 85%, 90%, 95%, 99%, 99.5%, or 99.9% identity to SEQ ID NO: 17. In some embodiments, the fusion protein comprises the polypeptide sequence of GPS1.1-F9-MPT4 (SEQ ID NO: 18) or a polypeptide sequence having at least 75%, 80%, 85%, 90%, 95%, 99%, 99.5%, or 99.9% identity to SEQ ID NO: 18. In some embodiments, the fusion protein comprises the polypeptide sequence of GPS1.1-F10-MPT4 (SEQ ID NO: 19) or a polypeptide sequence having at least 75%, 80%, 85%, 90%, 95%, 99%, 99.5%, or 99.9% identity to SEQ ID NO: 19. In some embodiments, the fusion protein comprises the polypeptide sequence of GPS3-F11-MPT4 (SEQ ID NO: 20) or a polypeptide sequence having at least 75%, 80%, 85%, 90%, 95%, 99%, 99.5%, or 99.9% identity to SEQ ID NO: 20. In some embodiments, the fusion protein comprises the polypeptide sequence of GPS2-F11-MPT4 (SEQ ID NO: 21) or a polypeptide sequence having at least 75%, 80%, 85%, 90%, 95%, 99%, 99.5%, or 99.9% identity to SEQ ID NO: 21.

In some embodiments, the fusion protein comprises the polypeptide sequence of GPS1.1-F11-MPT4.1 (SEQ ID NO: 56) or a polypeptide sequence having at least 75%, 80%, 85%, 90%, 95%, 99%, 99.5%, or 99.9% identity to SEQ ID NO: 56. In some embodiments, the fusion protein comprises the polypeptide sequence of GPS1.1-F5-MPT4.1 (SEQ ID NO: 54) or a polypeptide sequence having at least 75%, 80%, 85%, 90%, 95%, 99%, 99.5%, or 99.9% identity to SEQ ID NO: 54. In some embodiments, the fusion protein comprises the polypeptide sequence of GPS1.1-F9-MPT4.1 (SEQ ID NO: 53) or a polypeptide sequence having at least 75%, 80%, 85%, 90%, 95%, 99%, 99.5%, or 99.9% identity to SEQ ID NO: 53. In some embodiments, the fusion protein comprises the polypeptide sequence of GPS1.1-F10-MPT4.1 (SEQ ID NO: 55) or a polypeptide sequence having at least 75%, 80%, 85%, 90%, 95%, 99%, 99.5%, or 99.9% identity to SEQ ID NO: 55. In some embodiments, the fusion protein comprises the polypeptide sequence of GPS3-F11-MPT4.1 (SEQ ID NO: 57) or a polypeptide sequence having at least 75%, 80%, 85%, 90%, 95%, 99%, 99.5%, or 99.9% identity to SEQ ID NO: 57. In some embodiments, the fusion protein comprises the polypeptide sequence of GPS2-F11-MPT4.1 (SEQ ID NO: 58) or a polypeptide sequence having at least 75%, 80%, 85%, 90%, 95%, 99%, 99.5%, or 99.9% identity to SEQ ID NO: 58.

In some embodiments, the fusion protein comprises the polypeptide sequence of GPS1.1-F16-APT73.74 (SEQ ID NO: 59) or a polypeptide sequence having at least 75%, 80%, 85%, 90%, 95%, 99%, 99.5%, or 99.9% identity to GPS1.1-F16-APT73.74 (SEQ ID NO: 59).

In some embodiments, the fusion protein comprises the polypeptide sequence of APT73.74-F17-GPS1.1 (SEQ ID NO: 60) or a polypeptide sequence having at least 75%, 80%, 85%, 90%, 95%, 99%, 99.5%, or 99.9% identity to APT73.74-F17-GPS1.1 (SEQ ID NO: 60).

In some embodiments, the fusion protein comprises the polypeptide sequence of GPS1.1-F18-APT73.74 (SEQ ID NO: 61) or a polypeptide sequence having at least 75%, 80%, 85%, 90%, 95%, 99%, 99.5%, or 99.9% identity to GPS1.1-F18-APT73.74 (SEQ ID NO: 61).

In some embodiments, the fusion protein comprises the polypeptide sequence of APT73.74-F18-GPS1.1 (SEQ ID NO: 62) or a polypeptide sequence having at least 75%, 80%, 85%, 90%, 95%, 99%, 99.5%, or 99.9% identity to APT73.74-F18-GPS1.1 (SEQ ID NO: 62).

In some embodiments, the fusion protein comprises the polypeptide sequence of GPS1.1-F16-APT73.77 (SEQ ID NO: 63) or a polypeptide sequence having at least 75%, 80%, 85%, 90%, 95%, 99%, 99.5%, or 99.9% identity to GPS1.1-F16-APT73.77 (SEQ ID NO: 63).

In some embodiments, the fusion protein comprises the polypeptide sequence of APT73.77-F17-GPS1.1 (SEQ ID NO: 64) or a polypeptide sequence having at least 75%, 80%, 85%, 90%, 95%, 99%, 99.5%, or 99.9% identity to APT73.77-F17-GPS1.1 (SEQ ID NO: 64).

In some embodiments, the fusion protein comprises the polypeptide sequence of GPS1.1-F18-APT73.77 (SEQ ID NO: 65) or a polypeptide sequence having at least 75%, 80%, 85%, 90%, 95%, 99%, 99.5%, or 99.9% identity to GPS1.1-F18-APT73.77 (SEQ ID NO: 65).

In some embodiments, the fusion protein comprises the polypeptide sequence of APT73.77-F18-GPS1.1 (SEQ ID NO: 66) or a polypeptide sequence having at least 75%, 80%, 85%, 90%, 95%, 99%, 99.5%, or 99.9% identity to APT73.77-F18-GPS1.1 (SEQ ID NO: 66).

In some embodiments, the fusion protein comprises the polypeptide sequence of GPS1.1-F16-APT89.38 (SEQ ID NO: 67) or a polypeptide sequence having at least 75%, 80%, 85%, 90%, 95%, 99%, 99.5%, or 99.9% identity to GPS1.1-F16-APT89.38 (SEQ ID NO: 67).

In some embodiments, the fusion protein comprises the polypeptide sequence of APT89.38-F17-GPS1.1 (SEQ ID NO: 68) or a polypeptide sequence having at least 75%, 80%, 85%, 90%, 95%, 99%, 99.5%, or 99.9% identity to APT89.38-F17-GPS1.1 (SEQ ID NO: 68).

In some embodiments, the fusion protein comprises the polypeptide sequence of GPS1.1-F18-APT89.38 (SEQ ID NO: 69) or a polypeptide sequence having at least 75%, 80%, 85%, 90%, 95%, 99%, 99.5%, or 99.9% identity to GPS1.1-F18-APT89.38 (SEQ ID NO: 69).

In some embodiments, the fusion protein comprises the polypeptide sequence of APT89.38-F18-GPS1.1 (SEQ ID NO: 70) or a polypeptide sequence having at least 75%, 80%, 85%, 90%, 95%, 99%, 99.5%, or 99.9% identity to APT89.38-F18-GPS1.1 (SEQ ID NO: 70).

In some embodiments, the fusion protein further comprises a polypeptide having polyketide cyclase activity. In some embodiments, the addition of a polypeptide having polyketide cyclase activity to a polypeptide having prenyltransferase activity and a polypeptide having geranyl diphosphate synthase activity, results in the formation of a triple fusion protein. The polypeptide having PKC activity is not limited and may be any suitable polypeptide in the art. In some embodiments, the polypeptide having PKC activity is a PKC described in a separate filing (Attorney Docket No: CELB-002-WO1, filed on the same day as the present application), incorporated by reference in its entirety. As used herein, “PKC activity” refers to ability to cyclize a polyketide (i.e tetraketide) to an aromatic hydroxy acid (e.g., olivetolic acid or divarinic acid). In a preferred embodiment, the polypeptide having polyketide cyclase activity is a polypeptide with OA cyclase or DVA cyclase activities. In some embodiments, the polypeptide having polyketide cyclase activity comprises a polypeptide sequence having at least 70% identity to the polypeptide sequence of PKC1.0 (SEQ ID NO: 106), PKC1.1 (SEQ ID NO: 107), PKC4.33 (SEQ ID NO 108) or PKC11 (SEQ ID NO:109), or a functional fragment thereof. In other embodiments, the polypeptide having polyketide cyclase activity comprises a polypeptide sequence having at least 90% identity to SEQ ID NO: 107 (PKC1.1) or SEQ ID NO: 108 (PKC4.33) or PKC11 (SEQ ID NO:109). In still further embodiments, the polypeptide having polyketide cyclase activity is PKC1.1 (SEQ ID NO: 107) or PKC4.33 (SEQ ID NO: 108) or PKC11 (SEQ ID NO:109). In some embodiments, the polypeptide having polyketide cyclase activity has a polypeptide sequence having at least 75%, 80%, 85%, 90%, 95%, 99%, 99.5%, or 99.9% identity to of PKC1.0 (SEQ ID NO: 106), PKC1.1 (SEQ ID NO: 107), or PKC4.33 (SEQ ID NO 108) or PKC11 (SEQ ID NO:109).

In some embodiments, the PKC has at least 10%, 15%, 20%, 25%, 30%, 35%, 40%, 45%, 50%, 55%, 60%, 65%, 70%, 75%, 80%, 85%, 90%, 95%, or substantially 100% of the PKC activity of a naturally occurring PKC (e.g., PKC1 from Cannabis, PKC4 from Cannabis). In some embodiments, the PKC has at least 1.1-fold, 1.2-fold, 1.3-fold, 1.4-fold, 1.5-fold, 1.6-fold, 1.7-fold, 1.8-fold, 1.9-fold, 2-fold, 2.5-fold, 5-fold, 10-fold, or more PKC activity as compared to a naturally occurring PKC (e.g., PKC from Cannabis).

In some embodiments, the fusion protein comprising all three of the polypeptides having polyketide cyclase activity, prenyltransferase activity and geranyl diphosphate synthase activity, exhibits improved production of CBGA and/or CBGVA as compared to a control membrane bound prenyltransferase. In some embodiments, the fusion protein comprising all three of the polypeptides having polyketide cyclase activity, prenyltransferase activity and geranyl diphosphate synthase activity, exhibits improved production of CBGA and/or CBGVA as compared to a fusion protein comprising polypeptides having prenyltransferase and geranyl diphosphate synthase activities, but which lacks a polypeptide having polyketide cyclase activity.

In some embodiments, the fusion protein comprises, in order from the C-terminus, a polypeptide having PKC activity, a polypeptide having GPS activity, and a polypeptide having MPT activity (e.g., PKC-GPS-MPT). In some embodiments, the fusion protein comprises, in order from the C-terminus, a polypeptide having GPS activity, a polypeptide having PKC activity, and a polypeptide having MPT activity (e.g., GPS-PKC-MPT). In some embodiments, these fusion proteins further comprise one or more linkers between the polypeptides. The linkers are not limited and may be any linker disclosed herein.

In some embodiments, the fusion protein comprises the polypeptide sequence of GPS1.1-F11-PKC1.1-F11-MPT4 (SEQ ID NO: 92) or a polypeptide sequence having at least 75%, 80%, 85%, 90%, 95%, 99%, 99.5%, or 99.9% identity to GPS1.1-F11-PKC1.1-F11-MPT4 (SEQ ID NO: 92).

In some embodiments, the fusion protein comprises the polypeptide sequence of GPS1.1-F11-PKC1.1-F11-MPT4.1 (SEQ ID NO: 93) or a polypeptide sequence having at least 75%, 80%, 85%, 90%, 95%, 99%, 99.5%, or 99.9% identity to GPS1.1-F11-PKC1.1-F11-MPT4.1 (SEQ ID NO: 93).

In some embodiments, the fusion protein comprises the polypeptide sequence of GPS1.1-F11-PKC1.1-F11-MPT21.3 (SEQ ID NO: 94) or a polypeptide sequence having at least 75%, 80%, 85%, 90%, 95%, 99%, 99.5%, or 99.9% identity to GPS1.1-F11-PKC1.1-F11-MPT21.3 (SEQ ID NO: 94).

In some embodiments, the fusion protein comprises the polypeptide sequence of GPS1.1-F11-PKC4.33-F11-MPT4.1 (SEQ ID NO: 95), or a polypeptide sequence having at least 75%, 80%, 85%, 90%, 95%, 99%, 99.5%, or 99.9% identity to GPS1.1-F11-PKC4.33-F11-MPT4.1 (SEQ ID NO: 95).

In some embodiments, the fusion protein comprises the polypeptide sequence of GPS1.1-F11-PKC4.33-F11-MPT21.3 (SEQ ID NO: 96) or a polypeptide sequence having at least 75%, 80%, 85%, 90%, 95%, 99%, 99.5%, or 99.9% identity to GPS1.1-F11-PKC4.33-F11-MPT21.3 (SEQ ID NO: 96).

In some embodiments, the fusion protein comprises the polypeptide sequence of GPS1.1-PKC4.33-MPT4.1 (SEQ ID NO: 97), or a polypeptide sequence having at least 75%, 80%, 85%, 90%, 95%, 99%, 99.5%, or 99.9% identity to GPS1.1-PKC4.33-MPT4.1 (SEQ ID NO: 97).

In some embodiments, the fusion protein comprises the polypeptide sequence of GPS1.1-PKC4.33-MPT21.3 (SEQ ID NO: 98), or a polypeptide sequence having at least 75%, 80%, 85%, 90%, 95%, 99%, 99.5%, or 99.9% identity to GPS1.1-PKC4.33-MPT21.3 (SEQ ID NO: 98),

In some embodiments, the fusion protein comprises the polypeptide sequence of PKC1.1-F11-GPS1.1-F11-MPT4.1 (SEQ ID NO: 99), or a polypeptide sequence having at least 75%, 80%, 85%, 90%, 95%, 99%, 99.5%, or 99.9% identity to PKC1.1-F11-GPS1.1-F11-MPT4.1 (SEQ ID NO: 99).

In some embodiments, the fusion protein comprises the polypeptide sequence of PKC4.33-F11-GPS1.1-F11-MPT4.1 (SEQ ID NO: 100), or a polypeptide sequence having at least 75%, 80%, 85%, 90%, 95%, 99%, 99.5%, or 99.9% identity to PKC4.33-F11-GPS1.1-F11-MPT4.1 (SEQ ID NO: 100),

In some embodiments, the fusion protein comprises the polypeptide sequence of PKC4.33-GPS1.1-F11-MPT4.1 (SEQ ID NO: 101), or a polypeptide sequence having at least 75%, 80%, 85%, 90%, 95%, 99%, 99.5%, or 99.9% identity to PKC4.33-GPS1.1-F11-MPT4.1 (SEQ ID NO: 101).

In some embodiments, the fusion protein comprises the polypeptide sequence of PKC1.1-F11-GPS1.1-F11-MPT21.3 (SEQ ID NO: 102), or a polypeptide sequence having at least 75%, 80%, 85%, 90%, 95%, 99%, 99.5%, or 99.9% identity to PKC1.1-F11-GPS1.1-F11-MPT21.3 (SEQ ID NO: 102).

In some embodiments, the fusion protein comprises the polypeptide sequence of PKC4.33-F11-GPS1.1-F11-MPT21.3 (SEQ ID NO: 103), or a polypeptide sequence having at least 75%, 80%, 85%, 90%, 95%, 99%, 99.5%, or 99.9% identity to PKC4.33-F11-GPS1.1-F11-MPT21.3 (SEQ ID NO: 103).

In some embodiments, the fusion protein comprises the polypeptide sequence of PKC4.33-GPS1.1-F11-MPT21.3 (SEQ ID NO: 104), or a polypeptide sequence having at least 75%, 80%, 85%, 90%, 95%, 99%, 99.5%, or 99.9% identity to PKC4.33-GPS1.1-F11-MPT21.3 (SEQ ID NO: 104).

In some embodiments, the fusion protein comprises the polypeptide sequence of GPS1.1-F11-PKC1.1-F18-APT73.77 (SEQ ID NO: 110), or a polypeptide sequence having at least 75%, 80%, 85%, 90%, 95%, 99%, 99.5%, or 99.9% identity to GPS1.1-F11-PKC1.1-F18-APT73.77 (SEQ ID NO: 110).

In some embodiments, the fusion protein comprises the polypeptide sequence of GPS1.1-F11-PKC4.33-F18-APT73.77 (SEQ ID NO: 111), or a polypeptide sequence having at least 75%, 80%, 85%, 90%, 95%, 99%, 99.5%, or 99.9% identity to GPS1.1-F11-PKC4.33-F18-APT73.77 (SEQ ID NO: 111).

In some embodiments, the fusion protein comprises the polypeptide sequence of to GPS1.1-PKC4.33-APT73.77 (SEQ ID NO: 112), or a polypeptide sequence having at least 75%, 80%, 85%, 90%, 95%, 99%, 99.5%, or 99.9% identity to GPS1.1-PKC4.33-APT73.77 (SEQ ID NO: 112).

In some embodiments, the fusion protein comprises the polypeptide sequence of to GPS1.1-F11-PKC11-F11-MPT4 (SEQ ID NO: 113), or a polypeptide sequence having at least 75%, 80%, 85%, 90%, 95%, 99%, 99.5%, or 99.9% identity to GPS1.1-F11-PKC11-F11-MPT4 (SEQ ID NO: 113).

In some embodiments, the fusion protein comprises the polypeptide sequence of to GPS1.1-F11-PKC11-F11-MPT4.1 (SEQ ID NO: 114), or a polypeptide sequence having at least 75%, 80%, 85%, 90%, 95%, 99%, 99.5%, or 99.9% identity to GPS1.1-F11-PKC11-F11-MPT4.1 (SEQ ID NO: 114).

### Recombinant Cells and Cell Culture

Some aspects of the present disclosure are directed to a cell expressing an rMPT as described herein. Some aspects of the present disclosure are directed to a cell having an exogenous nucleic acid sequence coding for an rMPT as described herein.

Some aspects of the present disclosure are directed to a cell expressing a fusion protein as described herein. Some aspects of the present disclosure are directed to a cell having an exogenous nucleic acid sequence coding for a fusion protein as described herein.

Some aspects of the present disclosure are directed in a cell is expressing exogenous membrane transporters that can increase the uptake of aromatic acids including OA and DVA. In some embodiments these enzymes include hydroxybenzoate importers from eukaryotic organisms such as Candida (HBT1, UNIPROT: G8BL03 and HBT2, UNIPROT: G8BG57) or bacterial such as Acinetobacter (pcaK, UNIPROT: Q43975 or benK, UNIPROT: O30513) Corynbacterium (genK, UNIPROT: Q8NLB7 or Cg12385, UNIPOROT: Q8NN28) and other transporters in this family.

Some aspects of the present disclosure are directed in the deactivation of native aromatic acid or related general exporters resulting in the reduction of the rate of OA and DVA export from the cell. In some embodiments exporters in Saccharomyces include, but are not limited to, pdr12 (UNIPROT: Q02785), TPO1 (UNIPROT: Q07824) or other native exporters active in OA and DVA. In other embodiments exporters in Yarrowia can be down-regulated, knocked-down, or be inactivated. These exporter genes include, but are not limited to, to UNIPROT proteins: Q6C745, Q6CCM3, Q6CGV6, Q6C2K7, Q6CGW3, Q6C2G6, Q6CCJ1, Q6C539, Q6C5V9, Q6C4F7, Q6C8C4 Q6C2G6, and homologs with more than 95% protein sequence identity.

The cell is not limited and may be any suitable cell for expression. In some embodiments, the cell may be a microorganism or a plant. In some embodiments, the microorganism is a bacteria (e.g., E. coli), an algae, or a yeast. In some embodiments, the yeast is an oleaginous yeast (e.g., a Yarrowia lipolytica strain). In some embodiments, the bacteria is Escherichia coli.

Suitable cells may include, but are not limited to, Pichia pastoris, Pichia finlandica, Pichia trehalophila, Pichia koclamae, Pichia membranaefaciens, Pichia opuntiae, Pichia thermotolerans, Pichia salictaria, Pichia guercuum, Pichia pijperi, Pichia stiptis, Pichia methanolica, Pichia sp., Saccharomyces cerevisiae, Saccharomyces sp., Hansenula polymorpha (now known as Pichia angusta), Kluyveromyces sp., Kluyveromyces lactis, Kluyveromyces marxianus, Schizosaccharomyces pompe, Dekkera bruxellensis, Arxula adeninivorans, Candida albicans, Aspergillus nidulans, Aspergillus niger, Aspergillus oryzae, Trichoderma reesei, Chrysosporium lucknowense, Fusarium sp., Fusarium gramineum, Fusarium venenatum, Neurospora crassa, Chlamydomonas reinhardtii, Yarrowia lipolytica and the like. In some embodiments, the cell is a protease-deficient strain of Saccharomyces cerevisiae. In some embodiments, the cell is a eukaryotic cell other than a plant cell. In some embodiments, the cell is a plant cell. In some embodiments, the cell is a plant cell, where the plant cell is one that does not normally produce a cannabinoid, a cannabinoid derivative or analogue, a cannabinoid precursor, or a cannabinoid precursor derivative or analogue. In some embodiments, the cell is Saccharomyces cerevisiae. In some embodiments, the cell disclosed herein is cultured in vitro.

In some embodiments, the cell is a prokaryotic cell. Suitable prokaryotic cells may include, but are not limited to, any of a variety of laboratory strains of Escherichia coli, Lactobacillus sp., Salmonella sp., Shigella sp., and the like. See, e.g., Carrier et al, (1992) J. Immunol. 148:1176-1181; U.S. Pat. No. 6,447,784; and Sizemore et al. (1995) Science 270:299-302. Examples of Salmonella strains which can be employed may include, but are not limited to, Salmonella typhi and S. typhimurium. Suitable Shigella strains may include, but are not limited to, Shigella flexneri, Shigella sonnei, and Shigella disenteriae. Typically, the laboratory strain is one that is non-pathogenic. Non-limiting examples of other suitable bacteria may include, but are not limited to, Bacillus subtilis, Pseudomonas putida, Pseudomonas aeruginosa, Pseudomonas mevalonii, Rhodobacter sphaeroides, Rhodobacter capsulatus, Rhodospirillum rubrum, Rhodococcus sp., and the like.

An expression vector or vectors can be constructed to include exogenous nucleotide sequences coding for the rMPT or APT described herein operably linked to expression control sequences functional in the cell. Expression vectors applicable include, for example, plasmids, phage vectors, viral vectors, episomes and artificial chromosomes, including vectors and selection sequences or markers operable for stable integration into a host chromosome. Additionally, the expression vectors can include one or more selectable marker genes and appropriate expression control sequences. Selectable marker genes also can be included that, for example, provide resistance to antibiotics or toxins, complement auxotrophic deficiencies, or supply critical nutrients not in the culture media. Expression control sequences can include constitutive and inducible promoters, transcription enhancers, transcription terminators, and the like which are well known in the art. When two or more exogenous encoding nucleic acids are to be co- expressed, both nucleic acids can be inserted, for example, into a single expression vector or in separate expression vectors. For single vector expression, the encoding nucleic acids can be operationally linked to one common expression control sequence or linked to different expression control sequences, such as one inducible promoter and one constitutive promoter. The transformation of exogenous nucleic acid sequences can be confirmed using methods well known in the art. Such methods include, for example, nucleic acid analysis such as Northern blots or polymerase chain reaction (PCR) amplification of mRNA, or immunoblotting for expression of gene products, or other suitable analytical methods to test the expression of an introduced nucleic acid sequence or its corresponding gene product. It is understood by those skilled in the art that the exogenous nucleic acid is expressed in a sufficient amount to produce the desired product, and it is further understood that expression levels can be optimized to obtain sufficient expression using methods well known in the art and as disclosed herein.

The term “exogenous” is intended to mean that the referenced molecule or the referenced activity is introduced into the cell. The molecule can be introduced, for example, by introduction of an encoding nucleic acid into the host genetic material such as by integration into a host chromosome or as non-chromosomal genetic material such as a plasmid. Therefore, the term as it is used in reference to expression of an encoding nucleic acid refers to introduction of the encoding nucleic acid in an expressible form into the cell. When used in reference to a biosynthetic activity, the term refers to an activity that is introduced into the host. The source can be, for example, a homologous or heterologous encoding nucleic acid that expresses the referenced activity following introduction into the cell. Therefore, the term “endogenous” refers to a referenced molecule or activity that is present in the cell. Similarly, the term when used in reference to expression of an encoding nucleic acid refers to expression of an encoding nucleic acid contained within the microbial organism. The term “heterologous” refers to a molecule or activity derived from a source other than the referenced species whereas “homologous” refers to a molecule or activity derived from the host microbial organism. Accordingly, exogenous expression of an encoding nucleic acid can utilize either or both a heterologous or homologous encoding nucleic acid.

In some embodiments, the cell expressing rMPT or APT is capable of producing CBGA in the presence of GPP and OA (e.g., as metabolically produced by the cell or through the presence of OA). In some embodiments, the cell expressing rMPT or APT is capable of producing CBGA from a carbon source. In some embodiments, the cell expressing rMPT or APT is capable of producing CBGA from a carbon source in the presence of hexanoic acid.

In some embodiments, the cell expressing rMPT or APT is capable of producing CBGVA in the presence of GPP and DVA (e.g., as metabolically produced by the cell or through the presence of one or more of DVA in the media). In some embodiments, the cell expressing rMPT or APT is capable of producing CBGVA from a carbon source. In some embodiments, the cell expressing rMPT or APT is capable of producing CBGVA from a carbon source in the presence of butyric acid.

In some embodiments, the cell expressing rMPT or APT is capable of making a cannabinoid or analog thereof in the presence of a carbon source and, optionally, hexanoic or butyric acid.

In some embodiments, the cell expressing the fusion protein is capable of producing CBGA in the presence of GPP and OA (e.g., as metabolically produced by the cell or through the presence of OA in the media). In some embodiments, the cell expressing the fusion protein is capable of producing CBGA from a carbon source. In some embodiments, the cell expressing the fusion protein is capable of producing CBGA from a carbon source in the presence of hexanoic acid.

In some embodiments, the cell expressing the fusion protein is capable of producing CBGVA in the presence of GPP and DVA (e.g., as metabolically produced by the cell or through the presence of DVA in the media). In some embodiments, the cell expressing the fusion protein is capable of producing CBGVA from a carbon source. In some embodiments, the cell expressing the fusion protein is capable of producing CBGVA from a carbon source in the presence of butyric acid.

In some embodiments, the cell expressing the fusion protein is capable of making a cannabinoid or analog thereof in the presence of a carbon source and, optionally, hexanoic or butyric acid. Exemplary carbon sources include sugar carbons such as sucrose, glucose, mannitol, galactose, fructose, mannose, isomaltose, xylose, pannose, maltose, arabinose, cellobiose and 3-, 4-, or 5- oligomers thereof. Other carbon sources include alcohol carbon sources such as, ethanol, glycerol. Other carbon sources may contain a combination of the above carbon sources such as, for example, glucose/mannitol or glucose/ethanol. Other carbon sources include acid and esters such as acetate or formate, or fatty acids having four to twenty-two carbon atoms or fatty acid esters thereof. Other carbon sources can include renewal feedstocks and biomass. Exemplary renewal feedstocks include cellulosic biomass, hemicellulosic biomass and lignin feedstocks. Mixed carbon sources can also be used, such as a fatty acid and a sugar as described herein.

Depending on the cell, the appropriate culture medium may be used. For example, descriptions of various culture media may be found in “Manual of Methods for General Bacteriology” of the American Society for Bacteriology (Washington D.C., USA, 1981). As used here, “medium” as it relates to the growth source refers to the starting medium be it in a solid or liquid form. “Cultured medium”, on the other hand and as used here refers to medium (e.g. liquid medium) containing microbes that have been fermentatively grown and can include other cellular biomass. The medium generally includes one or more carbon sources, nitrogen sources, inorganic salts, vitamins and/or trace elements.

The culture conditions can include, for example, liquid culture procedures as well as fermentation and other large-scale culture procedures. Useful yields of the products can be obtained under aerobic culture conditions. An exemplary growth condition for achieving, one or more cannabinoid products includes aerobic culture or fermentation conditions. In certain embodiments, the microbial organism can be sustained, cultured or fermented under aerobic conditions.

Substantially aerobic conditions include, for example, a culture, batch fermentation or continuous fermentation such that the dissolved oxygen concentration in the medium remains between 5% and 100% of saturation. The percent of dissolved oxygen can be maintained by, for example, sparging air, pure oxygen or a mixture of air and oxygen.

The culture conditions can be scaled up and grown continuously for manufacturing cannabinoid product. Exemplary growth procedures include, for example, fed-batch fermentation and batch separation; fed-batch fermentation and continuous separation, or continuous fermentation and continuous separation. All of these processes are well known in the art. Fermentation procedures are particularly useful for the biosynthetic production of commercial quantities of cannabinoid product. Generally, and as with non-continuous culture procedures, the continuous and/or near-continuous production of cannabinoid product will include culturing a cannabinoid producing organism on sufficient nutrients and medium to sustain and/or nearly sustain growth in an exponential phase. Continuous culture under such conditions can include, for example, 1 day, 2, 3, 4, 5, 6 or 7 days or more. Additionally, continuous culture can include 1 week, 2, 3, 4 or 5 or more weeks and up to several months. Alternatively, the desired microorganism can be cultured for hours, if suitable for a particular application. It is to be understood that the continuous and/or near-continuous culture conditions also can include all time intervals in between these exemplary periods. It is further understood that the time of culturing the microbial organism is for a sufficient period of time to produce a sufficient amount of product for a desired purpose.

Fermentation procedures are well known in the art. Briefly, fermentation for the biosynthetic production of cannabinoid product can be utilized in, for example, fed-batch fermentation and batch separation; fed-batch fermentation and continuous separation, or continuous fermentation and continuous separation. Examples of batch and continuous fermentation procedures are well known in the art.

In some embodiments, the method comprises providing a cell as described herein comprising an exogenous nucleotide sequence coding for a rMPT or APT as described herein and culturing the cell to produce a cannabinoid, cannabinoid derivative, or cannabinoid analogue thereof.

In some embodiments, the method comprises providing a cell as described herein comprising an exogenous nucleotide sequence coding for a fusion protein described herein and culturing the cell to produce a cannabinoid, cannabinoid derivative, or cannabinoid analogue thereof. In some embodiments, the method comprises providing a cell as described herein comprising an exogenous nucleotide sequence coding for a fusion protein described herein and culturing the cell to produce the cannabinoid or analogue thereof.

In some embodiments, the method comprises providing a cell as described herein comprising an exogenous nucleotide sequence coding for an OA or DVA importer protein described herein and culturing the cell to produce a cannabinoid, cannabinoid derivative, or cannabinoid analogue thereof. In some embodiments, the method comprises providing a cell as described herein comprising an exogenous nucleotide sequence coding for an OA or DVA importer protein described herein and culturing the cell to produce the cannabinoid or analogue thereof.

In some embodiments, the method comprises providing a cell as described herein comprising an inactivated ore deleted nucleotide sequence coding for an OA or DVA exporter protein described herein and culturing the cell to produce a cannabinoid, cannabinoid derivative, or cannabinoid analogue thereof. In some embodiments, the method comprises providing a cell as described herein comprising an inactivated or deleted nucleotide sequence coding for an OA or DVA exporter protein described herein and culturing the cell to produce the cannabinoid or analogue thereof.

The cannabinoids, cannabinoid derivatives and cannabinoid analogues produced by the methods disclosed herein are not limited and may be any disclosed cannabinoid. In some embodiments, the cannabinoids, cannabinoid derivatives and cannabinoid analogues are selected from cannabigerolic acid, tetrahydrocannabinolic acid, tetrahydrocannabinol, cannabidiolic acid, cannabidiol, cannabigerol, cannabichromenic acid, cannabichromene, or an acid or derivative or analogue thereof.

In some embodiments, the methods further comprise a step of purifying or isolating the cannabinoids, derivatives or analogues thereof from the culture. Methods of isolation are not limited and may be any suitable method known in the art. Purification methods include, for example, extraction procedures (e.g., using supercritical carbon dioxide, ethanol or mixtures of these two), as well as methods that include continuous liquid-liquid extraction, pervaporation, evaporation, filtration, membrane filtration (including reverse osmosis, nanofiltration, ultrafiltration, and microfiltration), membrane filtration with diafiltration, membrane separation, reverse osmosis, electrodialysis, distillation, extractive distillation, reactive distillation, azeotropic distillation, crystallization and recrystallization, centrifugation, extractive filtration, ion exchange chromatography, size exclusion chromatography, adsorption chromatography, carbon adsorption, hydrogenation, and ultrafiltration or centrifugal partition chromatography (CPC).

In some embodiments, the cells are grown in stirred tank fermenters with feed supplementation (sugars with or without organic acids) where the dissolved oxygen, temperature, and pH are be controlled according to the optimal growth and production process. In some embodiments, aqueous non-miscible organic solvents are supplemented to dissolve added organic acids or extract the cannabinoid products as they are being synthesized. In some embodiments, these solvents may include, but are not limited to, isopropyl myristate (IPM), diisobutyl adipate, Bis(2-ethylhexyl) adipate, decane, dodecane, hexadecane or anther organic solvent with log P>5. The later number (log P) is defined as the log of a compound's partition between water and octanol and is a standard parameter of a compound's hydrophobicity (the larger the log P the less soluble in water). Depending on the fermentation process, the products can be isolated and purified using different methods.

If no organic cosolvent is used the targeted cannabinoid(s) precipitate together with the cell biomass after centrifugation, or is isolated in the solids after water removal using spray drying or other methods that remove water (i.e lyophilization, ultrafiltration etc). In one embodiment, an aqueous miscible organic solvent (ethanol, acetonitrile, etc.) is added to the cannabinoid containing cell pellet to dissolve the products. In some embodiments, a simple filtration, ultrafiltration or centrifugation can remove the cells and the aqueous/organic media evaporated to dryness or to a small volume from which the cannabinoid product will precipitate or crystalize. Alternatively, the cannabinoid containing cell pellet can be extracted with an aqueous immiscible organic solvent (ethyl acetate, heptane, decane, etc.) or supercritical carbon dioxide (with 0-10% Ethanol) to extract the cannabinoids. Evaporation of the organic solvent and a possible recrystallization will produce pure cannabinoid. If the cannabinoid products are not extracted by the previous methods and are trapped inside the cell, cells lysis may be required prior to extraction methods described above. In some embodiments, cells are disrupted using mechanical methods or by suspension in appropriate lysis buffers from which the cannabinoids can be extracted with an organic aqueous immiscible solvent (ethyl acetate, hexane, decane, methylene chloride, etc.). In other embodiments, cells may be suspended in an organic solvent (ethanol, methanol, methylene chloride, etc.) that extracts the cannabinoids from the cells.

In some embodiments, an organic solvent is required during growth that is separated at the end of the fermentation. Back extraction with alkaline aqueous solvent or a different organic solvent with low boiling point and high polarity (ethanol, acetonitrile, etc.) will remove the cannabinoids. Isolation can then involve a simple pH shift if water is used, or an evaporation if organic solvents are used. In both cases, a recrystallization step may be required at the end to improve purity of the product.

### EXAMPLES

### Example 1: MPT Engineering Strategy

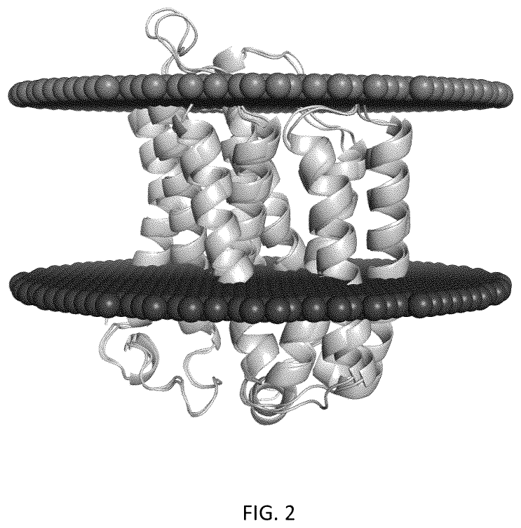

To create new novel sequences with improved properties, MPT1 and MPT4 were recombined. We used a structure-guided recombination approach like what has been described in the literature (Arnold F H, et al PNAS, 2017, 114(13), E2624) that recombines proteins based on their structural similarities. The first step of this approach required the development of structural models of both MPT1 and MPT4. These structural models and their alignment were created as described below and are shown in FIG. 2. Guided by these structural models, 160 novel chimeric sequences were designed by recombining sequence elements of MPT1 and MPT4, synthetic genes for each chimeric protein were synthesized, were cloned in a plasmid vector (pCL-SE-0337), transformed in Yarrowia and were screened for CBGA or CBGVA production in 96 well plates as described in Example 2. Three top hits were identified, MPT21, MPT26 and MPT31. The plasmids containing these mutants were isolated, re-sequenced and were transformed in fresh Yarrowia strain for re-screening (Example 2)

TABLE 1
sequence identity of rMPTs discovered

| | MPT4.1 | MPT21 | MPT26 | MPT31 |
|---|---|---|---|---|
| MPT4.1 | | 90.9 | 90.6 | 87.7 |
| MPT21 | 90.9 | | 93.2 | 90.3 |
| MPT26 | 90.6 | 93.2 | | 90.6 |
| MPT31 | 87.7 | 90.3 | 90.6 | |

### Example 2—Screening of MPT Library Hits for CBGA and CBGVA Activity: OA and DVA Feed

As described in detail below, plasmid pCL-SE-0337.MPT21,26,31 were transformed into strain sCL-SE-0128 and 4 separate colonies of each were patched and precultured for 48 h. Assay cultures consisted of minimal media with 100 mM MES pH 6.5, 0.5% ethanol and 2 mM olivetolic or divarinic acid (DVA). Cultures with olivetolic acid were quenched with equal volume of ethanol after 48 h total growth while cultures with DVA with quenched after 96 h total growth. Enzymes produced CBGA and FCBGA from olivetolic acid and CBGVA and FCBGVA from DVA. Averages and standard deviations were calculated from replicates.

TABLE 2
CBGA and CBGVA formation by MPT21, MPT26 and MPT31.
Products in μM accumulated in the in vivo assay.

| | OA Feed (48 h) | | DVA Feed (96 h) | |
|---|---|---|---|---|
| | | CBGA/ | | CBGVA/ |
| Strain | CBGA | FCBGA | CBGVA | FCBGVA |
| sCL-SE-0128 + | 310 ± 11 | 3.1 | 647 ± 45 | 6.0 |
| pCL-SE-0337.MPT21 | | | | |
| sCL-SE-0128 + | 261 ± 62 | 4.5 | 620 ± 134 | 13.7 |
| pCL-SE-0337.MPT26 | | | | |
| sCL-SE-0128 + | 175 ± 63 | 13.9 | 260 ± 79 | 22.6 |
| pCL-SE-0337.MPT31 | | | | |

The screening results confirmed the activity of the MPT mutants against both CBGA and CBGVA formation, and identified MPT21 as a particularly effective mutant for producing a robust titer of CBGA and CBGVA.

### Example 3: Engineering of MPTs for Further Improving Activity and Selectivity

To further improve the activity and selectivity of these enzymes further mutagenesis was performed. As described in below structure models were created and the substrate (OA and DVA) were docked in the active site. Based on these models, a number of amino acids were targeted for mutagenesis in each rMPT is shown in the following Table.

TABLE 3
mutagenesis of MPT4 and MPT21
MPT4 and MPT21

R22, P23, Y24, V25, V26, K27, G28, M29, S31, A33, F66, N67, A70,
A71, D80, I81, I84, N85, K86, P87, D88, L89, L91, V92, Y139,
S140, F154, L155, I158, S159, S160, V162, T194, V195, G197, M198,
I200, A201, F202, A203, K204, D208, I209, G211, D212, L267, A268,
L271, T275, L278, N282, A284, S285, R289, F292, I295, W296, L298,
Y299

### Example 4: Testing of Unfused rMPTs and sPTs for CBGA and CBGVA Formation

As described below, plasmids pCL-SE-0380 (MPT4), pCL-SE-0337.MPT4, pCL-SE-0338.APT73.74, and pCL-SE-0338.APT73.77 were transformed into strain sCL-SE-0128. Multiple colonies per transformation were precultured for 48 h in YNB containing glucose (2%), casamino acids (0.5%), and MES (100 mM pH 6.5). Pre-culture was used to inoculate assay medium comprised of YNB containing glucose (2%), casamino acids (0.5%), MES (100 mM pH 6.5), and olivetolic acid or divarinic acid (2 mM). Cultures were quenched with equal volume of ethanol after 48 h total growth and assayed for CBG(V)A and FCBG(V)A. Averages and standard deviations were calculated from replicates. The results are shown in the tables 4, 5 below:

TABLE 4
CBGA and FCBGA formation by MPT4, MPT4.1,
APT73.74 and APT73.77. Products in μM
accumulated in the in vivo assay (48 h quench)

| | Enzyme | | | CBGA/ |
|---|---|---|---|---|
| Strain | Expressed | CBGA | FCBGA | FCB [symbol] |
| sCL-SE-0128 + | MPT4 | 123 ± 13 | 23 ± 2 | 5.3 |
| pCL-SE-0380 | | | | |
| sCL-SE-0128 + | MPT4.1 | 168 ± 33 | 11 ± 2 | 14.6 |
| pCL-SE-0337.MPT4.1 | | | | |
| sCL-SE-0128 + | APT73.74 | 123 ± 5 | 18 ± 1 | 6.7 |
| pCL-SE-0338.APT73.74 | | | | |
| sCL-SE-0128 + | APT73.77 | 145 ± 13 | 55 ± 3 | 2.6 |
| pCL-SE-0338.APT73.77 | | | | |

[symbol]

indicates data missing or illegible when filed

TABLE 5
CBGVA and FCBGVA formation by MPT4, MPT4.1,
APT73.74 and APT73.77. Products in μM accumulated
in the in vivo assay (48 hour quench)

| | Enzyme | | | CBGVA/ |
|---|---|---|---|---|
| Strain | Expressed | CBGVA | FCBGVA | FCBGVA |
| sCL-SE-0128 + | MPT4 | 19 ± 2 | 0 ± 0 | NA |
| pCL-SE-0380 | | | | |
| sCL-SE-0128 + | MPT4.1 | 28 ± 8 | 0 ± 0 | NA |
| pCL-SE-0337.MPT4.1 | | | | |
| sCL-SE-0128 + | APT73.74 | 10 ± 0 | 0 ± 0 | NA |
| pCL-SE-0338.APT73.74 | | | | |
| sCL-SE-0128 + | APT73.77 | 10 ± 1 | 1 ± 1 | 12.5 |
| pCL-SE-0338.APT73.77 | | | | |

This example clearly shows that mutant MPT4.1 has both increased activity towards CBGA formation and produces less FCBGA byproduct compared to MPT4. Furthermore, APT73 mutants produce similar amounts of CBGA product but have different ratio of CBGA/FCBGA, with APT73.74 having better selectivity (CBGA/FCBGA) and APT73.77 having better overall activity (CBGA+FCBGA)

Interestingly, the much lower formation of CBGVA was produced by all enzymes, including the two rMPTs which contradicts the in vitro activity for OA and DVA described in Example 10. This difference is probably due to the difference of OA and DVA uptake by Yarrowia, the rate of export or a combination of both.

### Example 5: Comparison of MPT4.1 Activity With and Without Fusion With Erg20 and With MPT4 Fusion

As described below, plasmids pCL-SE-0337.MPT4.1 and pCL-SE-0753 were transformed into strain sCL-SE-0128. Multiple colonies per transformation were precultured for 24 h in YNB containing glucose (2%), casamino acids (0.5%), and MES (100 mM pH 6.5). Pre-culture was used to inoculate assay medium comprised of YNB containing glucose (6%), casamino acids (0.5%), MES (100 mM pH 6.5), and olivetolic acid (3 mM). Cultures were quenched with equal volume of ethanol after 48 h total growth and assayed for CBGA and FCBGA. Averages and standard deviations were calculated from replicates. The results are shown in the Table 6 below:

TABLE 6
CBGA and FCBGA formation by MPT4.1 and GPS1.1 fusions
with either MPT4 or MPT4.1. Products in μM accumulated
in the in vivo assay (48 h quench).

| | Enzyme | | | CBGA/ |
|---|---|---|---|---|
| Strain | Expressed | CBGA | FCBGA | FCBGA |
| sCL-SE-0128 + | MPT4.1 | 253 ± 18 | 26 ± 2 | 9.7 |
| pCL-SE-0337.MPT4.1 | | | | |
| sCL-SE-0128 + | GPS1.1-F11- | 425 ± 11 | 34 ± 1 | 12.6 |
| pCL-SE-0753 | MPT4.1 | | | |
| sCL-SE-0128 + | GPS1.1-F11- | 326 ± 16 | 62 ± 2 | 5.2 |
| pCL-SE-0406 | MPT4 | | | |

These results clearly show that MPT4.1 fusion of MPT4.1 with GPS1.1 increases both the titer of CBGA by 67% and the ratio of CBGA/FCBGA from 9.7 to 12.6. Furthermore MPT4.1 fusion outperforms the same fusion with MPT4 (similarly to unfused comparison described earlier).

### Example 6: Testing Linkers for Engineered GPS1.1 and MPT4 Fusion Proteins

As described in detail below, plasmids pCL-SE-0380 (MPT4), pCL-SE-0406, pCL-SE-0435, and pCL-SE-0437 were transformed into strain sCL-SE-0128. Four separate colonies per transformation were precultured for 24 h in YNB containing glycerol (2%), casamino acids (0.5%), and MES (100 mM pH 6.5). Pre-culture was used to inoculate assay medium comprised of YNB containing glycerol (2%), casamino acids (0.5%), MES (100 mM pH 6.5), and olivetolic acid (1 mM). Cultures were quenched after 72 h total growth by mixing with equal volume of EtOH. Enzymes produced CBGA and FCBGA. Averages and standard deviations were calculated from replicates. The results are shown in the table 7 below.

TABLE 7
CBGA and FCBGA formation by Erg20-MPT4 fusions
with different linkers. Products in μM accumulated
in the in vivo assay (72 h quench).

| | | | | CBGA/ |
|---|---|---|---|---|
| Strain | Enzyme Expressed | CBGA | FCBGA | FCBGA |
| sCL-SE-0128 + | MPT4 | 230 ± 15 | 51 ± 1 | 4.5 |
| pCL-SE-0380 | | | | |
| sCL-SE-0128 + | GPS1.1-F11-MPT4 | 393 ± 41 | 60 ± 5 | 6.6 |
| pCL-SE-0406 | | | | |
| sCL-SE-0128 + | GPS1.1-F5-MPT4 | 333 ± 129 | 49 ± 15 | 6.8 |
| pCL-SE-0435 | | | | |
| sCL-SE-0128 + | GPS1.1-F9-MPT4 | 338 ± 38 | 53 ± 1 | 6.3 |
| pCL-SE-0436 | | | | |
| sCL-SE-0128 + | GPS1.1-F10-MPT4 | 350 ± 17 | 54 ± 1 | 6.5 |
| pCL-SE-0437 | | | | |

sCL-SE-0128 does not contain prenyl-transferase activity. However, sCL-SE-0128 expresses HMGR and GPS1.1 to increase intracellular GPP levels for prenylation of OA to CBGA. The plasmids that were transformed contain either a gene expressing a prenyl-transferase (pCL-SE-0380 (MPT4)) or a gene expressing a fusion between GPS1.1 and a prenyl-transferase with different linker sequences. These results show that all fusions of MPT4 with GPS1.1, improve CBGA production and improve the ratio of CBGA to FCBGA.

### Example 7: Testing Fusions of Different GPSs With MPT4 (GPS-MPT4 Fusion Proteins)

As described below, plasmids pCL-SE-0337, 0406, 0452 and 0453 were transformed into strain sCL-SE-0128. Multiple colonies per transformation were precultured for 24 h in YNB containing glycerol (2%), casamino acids (0.5%), and MES (100 mM pH 6.5). Pre-culture was used to inoculate assay medium comprised of YNB containing glycerol (2%), casamino acids (0.5%), MES (100 mM pH 6.5), and olivetolic acid (1 mM). Cultures were quenched after 72 h total growth and assayed for CBGA and F-CBGA. Averages and standard deviations were calculated from replicates. The results are shown in the table 8 below:

TABLE 8
CBGA and FCBGA formation by MPT4 fusions with different GPSs.
Products in μM accumulated in the in vivo assay (72 h quench).

| | | | | CBGA/ |
|---|---|---|---|---|
| Strain | Enzyme Expressed | CBGA | FCBGA | FCBGA |
| sCL-SE-0128 + | None | 0 ± 0 | 0 ± 0 | |
| pCL-SE-0337 | | | | |
| sCL-SE-0128 + | GPS1.1-F11-MPT4 | 376 ± 41 | 51 ± 5 | 7.4 |
| pCL-SE-0406 | | | | |
| sCL-SE-0128 + | GPS3-F11-MPT4 | 255 ± 22 | 41 ± 3 | 6.2 |
| pCL-SE-0452 | | | | |
| sCL-SE-0128 + | GPS2-F11-MPT4 | 282 ± 69 | 11 ± 2 | 26.8 |
| pCL-SE-0453 | | | | |

sCL-SE-0128 does not contain prenyl-transferase activity. However, sCL-SE-0128 expresses HMGR and GPS1.1 to increase intracellular GPP levels for prenylation of OA to CBGA. The plasmids that were transformed contain a gene expressing a fusion between two different GPSs and a prenyl-transferase. The same linker is used for all constructs. The results above in combination with example 5 & 6 show that different GPSs can be successfully fused with MPTs) and that these fusions are a general approach to improve CBGA titers and/or the CBGA/ FCBGA ratio. Additionally, as illustrated by GPS2 (pCL-SE-0453), certain GPS-MPT combinations result in very high CBGA/ FCBGA ratios.

### Example 8—Fusion of Novel MPTs With GPS and OA Feed

As described below, plasmids pCL-SE-0406, pCL-SE-0664, pCL-SE-0663, pCL-SE-0662, pCL-SE-0380 (MPT4), pCL-SE-0337.MPT21, pCL-SE-0337.MPT26, and pCL-SE-0337.MPT31 were transformed into strain sCL-SE-0128. Multiple colonies per transformation were precultured for 48 h in YNB containing glucose (2%), casamino acids (0.5%), and MES (100 mM pH 6.5). Pre-culture was used to inoculate assay medium comprised of YNB containing glucose (2%), casamino acids (0.5%), MES (100 mM pH 6.5), and olivetolic acid (2 mM). Cultures were quenched with equal volume of ethanol after 48 h total growth and assayed for CBGA and FCBGA. Averages and standard deviations were calculated from replicates. The results are shown in the table 9 below:

TABLE 9
CBGA and FCBGA formation by MPT fusions with
GPS1.1 vs unfused MPTs. Products in μM
accumulated in the in vivo assay (48 h quench).

| | Enzyme | | | CBGA/ |
|---|---|---|---|---|
| Strain | Expressed | CBGA | FCBGA | FCBGA |
| sCL-SE-0128 + | GPS1.1- | 200 ± 3 | 24 ± 1 | 8.2 |
| pCL-SE-0406 | F11-MPT4 | | | |
| sCL-SE-0128 + | GPS1.1- | 156 ± 13 | 52 ± 5 | 3.0 |
| pCL-SE-0664 | F11-MPT21 | | | |
| sCL-SE-0128 + | GPS1.1- | 98 ± 8 | 21 ± 2 | 4.6 |
| pCL-SE-0663 | F11-MPT26 | | | |
| sCL-SE-0128 + | GPS1.1- | 53 ± 4 | 5 ± 0 | 10.8 |
| pCL-SE-0662 | F11-MPT31 | | | |
| sCL-SE-0128 + | MPT4 | 123 ± 13 | 23 ± 2 | 5.3 |
| pCL-SE-0380 | | | | |
| sCL-SE-0128 + | MPT21 | 109 ± 27 | 43 ± 10 | 2.6 |
| pCL-SE-0337.MPT21 | | | | |
| sCL-SE-0128 + | MPT26 | 61 ± 5 | 14 ± 1 | 4.4 |
| pCL-SE-0337.MPT26 | | | | |
| sCL-SE-0128 + | MPT31 | 40 ± 12 | 3 ± 2 | 13.4 |
| pCL-SE-0337.MPT31 | | | | |

As described below, plasmids pCL-SE-0337.GPS1.1.F11.MPT21, pCL-SE-0337.GPS1.1.L11.MPT21.1, pCL-SE-0337.GPS1.1.F11.MPT21.2, pCL-SE-0337.GPS1.1.F11.MPT21.3, pCL-SE-0337.GPS1.1.F11.MPT21.4, pCL-SE-0337.GPS1.1.F11.MPT21.5, pCL-SE-0337.GPS1.1.F11.MPT21.6, pCL-SE-0337.GPS1.1.F11.MPT21.7, pCL-SE-0337.GPS1.1.F11.MPT21.8, pCL-SE-0337.GPS1.1.F11.MPT21.9, pCL-SE-0337.GPS1.1.F11.MPT21.10, pCL-SE-0337.GPS1.1.F11.MPT21.11, pCL-SE-0337.GPS1.1.F11.MPT21.12, pCL-SE-0337.GPS1.1.F11.MPT21.13, pCL-SE-0337.GPS1.1.F11.MPT21.14, pCL-SE-0337.GPS1.1.F11.MPT21.15, pCL-SE-0337.GPS1.1.F11.MPT21.16, pCL-SE-0337.GPS1.1.F11.MPT21.17, pCL-SE-0337.GPS1.1.F11.MPT21.18, pCL-SE-0337.GPS1.1.F11.MPT21.19, pCL-SE-0337.GPS1.1.F11.MPT21.20, pCL-SE-0337.GPS1.1.F11.MPT21.22, and pCL-SE-0337.GPS1.1.F11.MPT4.1 were transformed into strain sCL-SE-0128. Multiple colonies per transformation were precultured for 48 h in YNB containing glucose (6%), casamino acids (0.5%), and MES (100 mM pH 6.5). Pre-culture was used to inoculate assay medium comprised of YNB containing glucose (6%), casamino acids (0.5%), MES (100 mM pH 6.5), and olivetolic acid (3 mM). Cultures were quenched with equal volume of ethanol after 48 h total growth and assayed for CBGA and FCBGA. Averages and standard deviations were calculated from replicates. The results are shown in the table 10 below:

TABLE 10
CBGA and FCBGA formation by MPT fusions with GPS1.1. Products
in μM accumulated in the in vivo assay (48 h quench).

| | | | | | C [symbol] |
|---|---|---|---|---|---|
| Strain | Enzyme Expressed | Mutation | CBGA | FCBGA | F [symbol] |
| sCL-SE-0128 + pCL-SE-0664 | GPS1.1-F11-MPT21 | | 276 ± 17 | 89 ± 4 | 3 [symbol] |
| sCL-SE-0128 + pCL-SE-0337.F11.MPT21.1 | GPS1.1-F11-MPT21.1 | Y24F | 236 ± 9 | 45 ± 1 | 5 [symbol] |
| sCL-SE-0128 + pCL-SE-0337.F11.MPT21.2 | GPS1.1-F11-MPT21.2 | Y24M | 202 ± 21 | 12 ± 1 | 1 [symbol] |
| sCL-SE-0128 + pCL-SE-0337.F11.MPT21.3 | GPS1.1-F11-MPT21.3 | V25A | 279 ± 30 | 35 ± 2 | 8 [symbol] |
| sCL-SE-0128 + pCL-SE-0337.F11.MPT21.4 | GPS1.1-F11-MPT21.4 | M29A | 207 ± 4 | 39 ± 1 | 5 [symbol] |
| sCL-SE-0128 + pCL-SE-0337.F11.MPT21.5 | GPS1.1-F11-MPT21.5 | S31T | 236 ± 3 | 43 ± 1 | 5 [symbol] |
| sCL-SE-0128 + pCL-SE-0337.F11.MPT21.6 | GPS1.1-F11-MPT21.6 | S31V | 174 ± 1 | 27 ± 0 | 6 [symbol] |
| sCL-SE-0128 + pCL-SE-0337.F11.MPT21.7 | GPS1.1-F11-MPT21.7 | S31A | 253 ± 24 | 72 ± 8 | 3 [symbol] |
| sCL-SE-0128 + pCL-SE-0337.F11.MPT21.8 | GPS1.1-F11-MPT21.8 | A71G | 223 ± 1 | 21 ± 0 | 1 [symbol] |
| sCL-SE-0128 + pCL-SE-0337.F11.MPT21.9 | GPS1.1-F11-MPT21.9 | A71S | 335 ± 17 | 54 ± 2 | 6 [symbol] |
| sCL-SE-0128 + pCL-SE-0337.F11.MPT21.10 | GPS1.1-F11-MPT21.10 | I81M | 242 ± 13 | 25 ± 1 | 9 [symbol] |
| sCL-SE-0128 + pCL-SE-0337.F11.MPT21.11 | GPS1.1-F11-MPT21.11 | I84V | 258 ± 9 | 67 ± 3 | 3 [symbol] |
| sCL-SE-0128 + pCL-SE-0337.F11.MPT21.12 | GPS1.1-F11-MPT21.12 | L155E | 27 ± 1 | 9 ± 0 | 3 [symbol] |
| sCL-SE-0128 + pCL-SE-0337.F11.MPT21.13 | GPS1.1-F11-MPT21.13 | S159A | 260 ± 12 | 69 ± 1 | 3 [symbol] |
| sCL-SE-0128 + pCL-SE-0337.F11.MPT21.14 | GPS1.1-F11-MPT21.14 | S159G | 287 ± 14 | 64 ± 1 | 4 [symbol] |
| sCL-SE-0128 + pCL-SE-0337.F11.MPT21.15 | GPS1.1-F11-MPT21.15 | S159V | 248 ± 1 | 65 ± 1 | 3 [symbol] |
| sCL-SE-0128 + pCL-SE-0337.F11.MPT21.16 | GPS1.1-F11-MPT21.16 | A201G | 225 ± 53 | 29 ± 5 | 7 [symbol] |
| sCL-SE-0128 + pCL-SE-0337.F11.MPT21.17 | GPS1.1-F11-MPT21.17 | T275Q | 249 ± 14 | 55 ± 2 | 4 [symbol] |
| sCL-SE-0128 + pCL-SE-0337.F11.MPT21.18 | GPS1.1-F11-MPT21.18 | T275A | 311 ± 12 | 77 ± 1 | 4 [symbol] |
| sCL-SE-0128 + pCL-SE-0337.F11.MPT21.19 | GPS1.1-F11-MPT21.19 | S285A | 253 ± 12 | 85 ± 3 | 3 [symbol] |
| sCL-SE-0128 + pCL-SE-0337.F11.MPT21.20 | GPS1.1-F11-MPT21.20 | S285G | 231 ± 1 | 46 ± 0 | 5 [symbol] |
| sCL-SE-0128 + pCL-SE-0337.F11.MPT21.22 | GPS1.1-F11-MPT21.22 | I295L | 296 ± 8 | 50 ± 2 | 5 [symbol] |
| sCL-SE-0128 + pCL-SE-0753 | GPS1.1-F11-MPT4.1 | | 358 ± 0 | 27 ± 0 | 1 [symbol] |

[symbol]

indicates data missing or illegible when filed

Mutagenesis of MPT21 identified enzymes with improved activity towards CBGA formation (i.e MPT21.9 & MPT21.18) and enzymes with significant activity and improved ratio of CBGA/FCBGA (i.e MPT21.2, MPT21.8 and MPT21.10). The mutations increasing activity and CBGA/FCBGA selectivity can be combined to further improve the performance of MPT21.

### Example 9: Fusion of Novel MPTs With GPS and DVA Feed

As described below, plasmids pCL-SE-0406, pCL-SE-0664, pCL-SE-0663, pCL-SE-0662, pCL-SE-0380 (MPT4), pCL-SE-0337.MPT21, were transformed into strain sCL-SE-0128. Multiple colonies per transformation were precultured for 48 h in YNB containing glucose (2%), casamino acids (0.5%), and MES (100 mM pH 6.5). Pre-culture was used to inoculate assay medium comprised of YNB containing glucose (2%), casamino acids (0.5%), MES (100 mM pH 6.5), and divarinic acid (2 mM). Cultures were quenched after 48 h total growth and assayed for CBGVA and FCBGVA. Averages and standard deviations were calculated from replicates. The results are shown in the table 11 below:

TABLE 11
CBGVA and FCBGVA formation by MPT fusions with GPS1.1 vs unfused
MPTs. Products in μM accumulated in the in vivo assay.

| Strain | Enzyme Expressed | CBGVA | FCBGVA | CBGVA/FCB [symbol] |
|---|---|---|---|---|
| sCL-SE-0128 + pCL-SE-0406 | GPS1.1-F11-MPT4 | 45 ± 2 | 0 ± 0 | NA |
| sCL-SE-0128 + pCL-SE-0664 | GPS1.1-F11-MPT21 | 28 ± 2 | 4 ± 0 | 7.2 |
| sCL-SE-0128 + pCL-SE-0380 | MPT4 | 19 ± 2 | 0 ± 0 | NA |
| sCL-SE-0128 + pCL-SE-0337.MPT21 | MPT21 | 9 ± 3 | 1 ± 1 | 10.8 |

[symbol]

indicates data missing or illegible when filed

As described below, plasmids pCL-SE-0337.GPS1.1.F11.MPT21, pCL-SE-0337.GPS1.1.L11.MPT21.1, pCL-SE-0337.GPS1.1.F11.MPT21.2, pCL-SE-0337.GPS1.1.F11.MPT21.3, pCL-SE-0337.GPS1.1.F11.MPT21.4, pCL-SE-0337.GPS1.1.F11.MPT21.5, pCL-SE-0337.GPS1.1.F11.MPT21.6, pCL-SE-0337.GPS1.1.F11.MPT21.7, pCL-SE-0337.GPS1.1.F11.MPT21.8, pCL-SE-0337.GPS1.1.F11.MPT21.9, pCL-SE-0337.GPS1.1.F11.MPT21.10, pCL-SE-0337.GPS1.1.F11.MPT21.11, pCL-SE-0337.GPS1.1.F11.MPT21.12, pCL-SE-0337.GPS1.1.F11.MPT21.13, pCL-SE-0337.GPS1.1.F11.MPT21.14, pCL-SE-0337.GPS1.1.F11.MPT21.15, pCL-SE-0337.GPS1.1.F11.MPT21.16, pCL-SE-0337.GPS1.1.F11.MPT21.17, pCL-SE-0337.GPS1.1.F11.MPT21.18, pCL-SE-0337.GPS1.1.F11.MPT21.19, pCL-SE-0337.GPS1.1.F11.MPT21.20, pCL-SE-0337.GPS1.1.F11.MPT21.22, and pCL-SE-0337.GPS1.1.F11.MPT4.1 were transformed into strain sCL-SE-0128. Multiple colonies per transformation were precultured for 48 h in YNB containing glucose (2%), casamino acids (0.5%), and MES (100 mM pH 6.5). Pre-culture was used to inoculate assay medium comprised of YNB containing glucose (2%), casamino acids (0.5%), MES (100 mM pH 6.5), and divarinic acid (2 mM). Cultures were quenched after 48 h total growth and assayed for CBGVA and FCBGVA. Averages and standard deviations were calculated from replicates. The results are shown in the table 12 below:

TABLE 12
CBGVA and FCBGVA formation by MPT fusions with GPS1.1.
Products in μM accumulated in the in vivo assay.

| | | | | C [symbol] / |
|---|---|---|---|---|
| | | | | F [symbol] |
| Strain | Enzyme Expressed | CBGVA | FCBGVA | A [symbol] |
| sCL-SE-0128 + pCL-SE-0664 | GPS1.1-F11-MPT21 | 58 ± 7 | 6 ± 1 | 9. [symbol] |
| sCL-SE-0128 + pCL-SE-0337.F11.MPT21.1 | GPS1.1-F11-MPT21.1 | 26 ± 2 | 0 ± 0 | N [symbol] |
| sCL-SE-0128 + pCL-SE-0337.F11.MPT21.2 | GPS1.1-F11-MPT21.2 | 13 ± 2 | 0 ± 0 | N [symbol] |
| sCL-SE-0128 + pCL-SE-0337.F11.MPT21.3 | GPS1.1-F11-MPT21.3 | 68 ± 12 | 0 ± 0 | N [symbol] |
| sCL-SE-0128 + pCL-SE-0337.F11.MPT21.4 | GPS1.1-F11-MPT21.4 | 20 ± 2 | 0 ± 0 | N [symbol] |
| sCL-SE-0128 + pCL-SE-0337.F11.MPT21.5 | GPS1.1-F11-MPT21.5 | 22 ± 2 | 0 ± 0 | N [symbol] |
| sCL-SE-0128 + pCL-SE-0337.F11.MPT21.6 | GPS1.1-F11-MPT21.6 | 11 ± 1 | 0 ± 0 | N [symbol] |
| sCL-SE-0128 + pCL-SE-0337.F11.MPT21.7 | GPS1.1-F11-MPT21.7 | 46 ± 12 | 1 ± 3 | 11 [symbol] |
| sCL-SE-0128 + pCL-SE-0337.F11.MPT21.8 | GPS1.1-F11-MPT21.8 | 22 ± 1 | 0 ± 0 | N [symbol] |
| sCL-SE-0128 + pCL-SE-0337.F11.MPT21.9 | GPS1.1-F11-MPT21.9 | 53 ± 5 | 0 ± 0 | N [symbol] |
| sCL-SE-0128 + pCL-SE-0337.F11.MPT21.10 | GPS1.1-F11-MPT21.10 | 36 ± 3 | 0 ± 0 | N [symbol] |
| sCL-SE-0128 + pCL-SE-0337.F11.MPT21.11 | GPS1.1-F11-MPT21.11 | 46 ± 5 | 1 ± 2 | 14 [symbol] |
| sCL-SE-0128 + pCL-SE-0337.F11.MPT21.12 | GPS1.1-F11-MPT21.12 | 0 ± 0 | 0 ± 0 | N [symbol] |
| sCL-SE-0128 + pCL-SE-0337.F11.MPT21.13 | GPS1.1-F11-MPT21.13 | 46 ± 3 | 0 ± 0 | N [symbol] |
| sCL-SE-0128 + pCL-SE-0337.F11.MPT21.14 | GPS1.1-F11-MPT21.14 | 60 ± 7 | 1 ± 2 | [symbol] |
| sCL-SE-0128 + pCL-SE-0337.F11.MPT21.15 | GPS1.1-F11-MPT21.15 | 39 ± 1 | 0 ± 0 | N [symbol] |
| sCL-SE-0128 + pCL-SE-0337.F11.MPT21.16 | GPS1.1-F11-MPT21.16 | 40 ± 9 | 0 ± 0 | N [symbol] |
| sCL-SE-0128 + pCL-SE-0337.F11.MPT21.17 | GPS1.1-F11-MPT21.17 | 34 ± 3 | 0 ± 0 | N [symbol] |
| sCL-SE-0128 + pCL-SE-0337.F11.MPT21.18 | GPS1.1-F11-MPT21.18 | 57 ± 5 | 3 ± 2 | 14 [symbol] |
| sCL-SE-0128 + pCL-SE-0337.F11.MPT21.19 | GPS1.1-F11-MPT21.19 | 48 ± 4 | 5 ± 0 | 10 [symbol] |
| sCL-SE-0128 + pCL-SE-0337.F11.MPT21.20 | GPS1.1-F11-MPT21.20 | 33 ± 2 | 0 ± 0 | N [symbol] |
| sCL-SE-0128 + pCL-SE-0337.F11.MPT21.22 | GPS1.1-F11-MPT21.22 | 45 ± 2 | 0 ± 0 | N [symbol] |
| sCL-SE-0128 + pCL-SE-0753 | GPS1.1-F11-MPT4.1 | 91 ± 6 | 0 ± 0 | N [symbol] |

[symbol]

indicates data missing or illegible when filed

### Example 10: In Vitro Activities of MPT4 and MPT21 With OA and DVA

Yarrowia cells sCL-SE-0128 expressing the fusion of GPS1.1 with MPT4 (pCL-SE-0406) and MPT21 (pCL-SE-0664) were grown at 30 mL culture at 30 C for 2 days in YNB with 20% glucose. The cells were collected by centrifugation, washed with homogenization buffer (MAK340-1KT Sigma Aldrich) and were resuspended in 2 mL of homogenization buffer. The suspended cells were transferred to 5 mL tubes containing 1 mL of glass beads and were homogenized by shaking for 5 min at max speed in a shaker (Retsch MM400). The lysed cells were transferred to new tubes and microsomes were prepared as described in the kit (MAK340-1KT). Activity assays were performed using microsomes from both enzymes and varying concentrations of OA and DVA.

TABLE 13
Kinetic properties of MPT4 and MPT21 with OA and DVA

| | OA | | DVA | |
|---|---|---|---|---|
| | K_{M} | k_{cat} | K_{M} | k_{cat} |
| Enzyme | (μM) | (μM/min) | (μM) | (μM/min) |
| GPS1.1-F11-MPT4 | 31.6 +/− 9.1 | 0.49 | 18.1 +/− 3.1 | 0.94 |
| GPS1.1-F11-MPT21 | 20.9 +/− 7.5 | 0.76 | 62.2 +/− 5.0 | 3.46 |

Although the activities for each substrate is not directly comparable between the two prenyl transferases because the enzymes are not purified to homogeneity, the relative activities of each enzyme for OA vs DVA can be compared. In both cases, the activity towards DVA is similar or higher than that of OA, something that is not observed in in vivo assays. This suggests that intracellular concentration of OA and DVA is responsible for the in vivo differences. The later is defined by the rate of uptake of the OA vs DVA vs the rate of export from the cell. This effect is being addressed by overexpression of OA and DVA putative import proteins, as well as inactivation of native Yarrowia exporters.

### Example 11: Testing of GPS-APT Fusions With OA Feed

Soluble prenyl transferase APT73.77 was fused with GPS1.1 at the N- or C-terminus with different linkers creating the plasmids shown in Table 14. These fusion proteins were expressed and screened as described in Example 8 (olivetolic acid feed)

TABLE 14
CBGA and FCBGA formation byAPT73.77 and GPS1.1 fusions with either APT73.77
or MPT4.1. Products in μM accumulated in the in vivo assay (48 h quench).

| Strain | Enzyme(s) Expressed | CBGA | FCBGA | CBGA/FCBG [symbol] |
|---|---|---|---|---|
| sCL-SE-0128 + pCL-SE-0753 | GPS1.1-F11-MPT4.1 | 365 ± 22 | 26 ± 1 | 14.3 |
| sCL-SE-0128 + pCL-SE-0338.APT73.77 | APT73.77 | 215 ± 11 | 32 ± 1 | 6.8 |
| sCL-SE-0128 + pCL-SE-0757 | APT73.77-F17-GPS1.1 | 242 ± 36 | 73 ± 9 | 3.3 |
| sCL-SE-0128 + pCL-SE-0776 | APT73.77-F18-GPS1.1 | 382 ± 26 | 149 ± 4 | 2.6 |
| sCL-SE-0128 + pCL-SE-0777 | GPS1.1-F16-APT73.77 | 421 ± 44 | 113 ± 12 | 3.7 |
| sCL-SE-0128 + pCL-SE-0778 | GPS1.1-F18-APT73.77 | 468 ± 24 | 121 ± 2 | 3.9 |

[symbol]

indicates data missing or illegible when filed

Both N- and C-terminal fusions of APT73.77 with GPS1.1 increased the flux of GPP and FPP from GPS1.1 to APT73.77, resulting in an increase of the final titers of CBGA and FCBGA. A similar effect was also observed when fusing membrane bound prenyl-transferases with GPS1.1 as shown in Examples 8 and 9. The linker sequence plays an important role in the final CBGA (and FCBGA) titers particularly when GPS1.1 is fused at its C-terminus with a soluble or membrane bound prenyltransferase. Further optimization of the linker sequences can further improve the activity of APT73 and its mutants.

### Example 12: Expression of Import/Export Proteins

As discussed in the previous examples, there is a discrepancy between the in vivo and the in vitro activities of MPT4 and MPT4.1 for DVA. Even though both enzymes have at least the same or better activity in in vitro assays for DVA compared to OA, and similar Km, they are both making much less CBGVA in vivo. One plausible explanation is that DVA is either entering the cell with much lower efficiency compared to OA, or that it is been secreted from the cell with higher efficiency than OA. It is not clear if OA and DVA are entering the cell by diffusion or are assisted by membrane importers.

In either case, overexpression of membrane importers for these enzymes should improve the intracellular concentration of DVA, and most likely that of OA, and therefore increase their conversion to CBGVA and CBGA. Membrane importers that are specific for aromatic acids and hydroxy-acids are known and have been identified in bacteria and yeasts (for example: Cillingova A, et al Sci Rep 2017, 7(1): 8998; Pernstich C, et al Protein Expr. Purif 2014, 101(100):68). Expression an importer with activity on OA and DVA in Yarrowia should increase the uptake and the titers of cannabinoid product. Even though import of OA and DVA may be diffusion controlled, the export of these compounds from the cell is most likely catalyzed by one of the many export proteins in yeasts and Yarrowia. A number of general exporter proteins have been identified in yeast some with ability to export a wide range of aromatic and hydrophobic molecules (for example pdr12: Nygard Y at al Yeast, 2014, 31(6): 219-232 or TPO1: Godincho C P et al Appl Microbiol Biotechnol 2017 101(12):5005-5018). Homologs of these and other known exporters have been identified in Yarrowia, that will be inactivated.

In a recent example, the opposite approach was taken when two native exporter genes in Yarrowia were overexpressed and the organism's tolerance to propionic acid was increased (Park Y-K, Nicaud J-M Yeast, 2020 37(1): 131-140). Several genes encoding membrane integrated proteins with putative activity in export of chemicals have been identified in Yarrowia and will be systematically inactivated to identify which are responsible for OA and DVA export from the cell and increase the cannabinoid titers. These will include, but are not limited to, the following genes: UNIPROT: Q6C745, Q6CCM3, Q6CGV6, Q6C2K7, Q6CGW3, Q6C2G6, Q6CCJ1, Q6C539, Q6C5V9, Q6C4F7, Q6C8C4 Q6C2G6.

### Example 13: Testing of GPS-PKC-MPT Fusions With Hexanoic Acid Feed

Strain SB-00311 (HCS2, PKS1.1 and PKC1.1) was co-transformed with 1) a construct (pCL-SE-0274) for expression of HMGR and GPS1.1 and 2) a construct (pCL-SE-0406) for expression of a fusion protein that contains, from N- to C-terminus, GPS1.1 and MPT4 linked using the F11 linker sequence or a construct (pCL-SE-0474) for expression of a fusion protein that contains, from N- to C-terminus, GPS1.1, PKC1.1 and MPT4, each linked using the F11 linker sequence.

Transformants were assessed for CBGA production from hexanoic acid. Assay cultures consisted of YNBD+CAA media with 2.5 mM hexanoic acid. After 24 h, an additional 5 mM hexanoic acid was added to each assay culture. Cultures were quenched after 48 h total growth. Table 15 shows the average and standard deviation of accumulated CBGA (μM) produced from 44 transformants with the GPS1.1-F11-MPT4 double fusion and 34 transformants with the GPS1.1-F11-PKC1.1-F11-MPT4 triple fusion. As can be seen, the addition of PKC1.1 to GPS1.1 and MPT4 to form a triple fusion resulted in improved production of CBGA compared to the GPS1.1-MPT4 double fusion.

TABLE 15
CBGA production by GPS1.1 fusions with MPT4 via a F11 linker
and PKC1.1 fusions with both MPT4 and GPS1.1. Products
in μM accumulated in the in vivo assay (48 h quench).

| Strain | MPT Fusion Expressed | CBGA |
|---|---|---|
| SB-00311 + pCL-SE-0274 + | GPS1.1-F11-MPT4 | 55.9 ± 26.4 |
| pCL-SE-0406 | | |
| SB-00311 + pCL-SE-0274 + | GPS1.1-F11-PKC1.1- | 77.4 ± 32.4 |
| pCL-SE-0474 | F11-MPT4 | |

### EXPERIMENTAL METHODS

### Cloning Methods, Vectors and Strains

E. coli Expression Plasmids

Genes for each enzyme were optimized for expression in E. coli, synthesized (Codex DNA), and cloned into the pM264-c vector (ATUM). Genes were sequenced verified and then subcloned into the pD441-NHT expression vector (ATUM) with an N-terminal His tag and TEV protease cleavage site under control of the T5 promoter. Plasmids were transformed into chemically competent E. coli BL21(DE3) cells (NEB), plated on LB agar plates with 50 μg/mL kanamycin, and grown overnight at 37° C. Colony PCR was used to verify gene fragment insertion and positive colonies were inoculated into liquid LB media with 50 μg/mL kanamycin and grown overnight at 37° C. and then diluted with glycerol to create stocks containing 25% glycerol which were stored at −80° C.

### Yarrowia Expression Plasmids

Genes for each enzyme were optimized for expression in Yarrowia, synthesized (Codex DNA), and cloned into the pM264-c vector (ATUM). Genes were sequenced verified and then subcloned into the SapI sites of pCL-SE-0331, pCL-SE-0332, or pCL-SE-0337. Plasmids were transformed into chemically competent E. coli NEB 10-beta cells (NEB), plated on LB agar plates with 50 μg/mL kanamycin or 100 ug/ml carbenicillin, and grown overnight at 37° C. Colony PCR was used to verify gene fragment insertion and positive colonies were inoculated into liquid LB media with the appropriate antibiotic. Cultures were grown overnight at 33° C. and then used for isolating plasmid DNA (Qiagen).

TABLE 16
plasmids with genetic elements:

| Plasmid | Host | Key gene(s) expressed |
|---|---|---|
| pCL-SE-0331 | Yarrowia | |
| pCL-SE-0332 | Yarrowia | |
| pCL-SE-0337 | Yarrowia | |
| pCL-SE-0337.MPT21 | Yarrowia | MPT21 |
| pCL-SE-0664 | Yarrowia | GPS1.1-F11-MPT21 |
| pCL-SE-0274 | Yarrowia | HMGR, GPS1.1 |
| pCL-SE-0337.MPT4.1 | Yarrowia | MPT4.1 |
| pCL-SE-0380 | Yarrowia | MPT4 |
| pCL-SE-0406 | Yarrowia | GPS1.1-F11-MPT4 |
| pCL-SE-0474 | Yarrowia | GPS1.1-F11-PKC1.1-F11-MPT4 |
| pCL-SE-0753 | Yarrowia | GPS1.1-F11-MPT4.1 |
| pCL-SE-0435 | Yarrowia | GPS1.1-F5-MPT4 |
| pCL-SE-0436 | Yarrowia | GPS1.1-F9-MPT4 |
| pCL-SE-0437 | Yarrowia | GPS1.1-F10-MPT4 |
| pCL-SE-0452 | Yarrowia | GPS3-F11-MPT4 |
| pCL-SE-0453 | Yarrowia | GPS2-F11-MPT4 |
| pCL-SE- | Yarrowia | GPS1.1-F11-MPT21.1 |
| 0337.F11.MPT21.1 | | |
| pCL-SE- | Yarrowia | GPS1.1-F11-MPT21.2 |
| 0337.F11.MPT21.2 | | |
| pCL-SE- | Yarrowia | GPS1.1-F11-MPT21.3 |
| 0337.F11.MPT21.3 | | |
| pCL-SE- | Yarrowia | GPS1.1-F11-MPT21.4 |
| 0337.F11.MPT21.4 | | |
| pCL-SE- | Yarrowia | GPS1.1-F11-MPT21.5 |
| 0337.F11.MPT21.5 | | |
| pCL-SE- | Yarrowia | GPS1.1-F11-MPT21.6 |
| 0337.F11.MPT21.6 | | |
| pCL-SE- | Yarrowia | GPS1.1-F11-MPT21.7 |
| 0337.F11.MPT21.7 | | |
| pCL-SE- | Yarrowia | GPS1.1-F11-MPT21.8 |
| 0337.F11.MPT21.8 | | |
| pCL-SE- | Yarrowia | GPS1.1-F11-MPT21.9 |
| 0337.F11.MPT21.9 | | |
| pCL-SE- | Yarrowia | GPS1.1-F11-MPT21.10 |
| 0337.F11.MPT21.10 | | |
| pCL-SE- | Yarrowia | GPS1.1-F11-MPT21.11 |
| 0337.F11.MPT21.11 | | |
| pCL-SE- | Yarrowia | GPS1.1-F11-MPT21.12 |
| 0337.F11.MPT21.12 | | |
| pCL-SE- | Yarrowia | GPS1.1-F11-MPT21.13 |
| 0337.F11.MPT21.13 | | |
| pCL-SE- | Yarrowia | GPS1.1-F11-MPT21.14 |
| 0337.F11.MPT21.14 | | |
| pCL-SE- | Yarrowia | GPS1.1-F11-MPT21.15 |
| 0337.F11.MPT21.15 | | |
| pCL-SE- | Yarrowia | GPS1.1-F11-MPT21.16 |
| 0337.F11.MPT21.16 | | |
| pCL-SE- | Yarrowia | GPS1.1-F11-MPT21.17 |
| 0337.F11.MPT21.17 | | |
| pCL-SE- | Yarrowia | GPS1.1-F11-MPT21.18 |
| 0337.F11.MPT21.18 | | |
| pCL-SE- | Yarrowia | GPS1.1-F11-MPT21.19 |
| 0337.F11.MPT21.19 | | |
| pCL-SE- | Yarrowia | GPS1.1-F11-MPT21.20 |
| 0337.F11.MPT21.20 | | |
| pCL-SE- | Yarrowia | GPS1.1-F11-MPT21.22 |
| 0337.F11.MPT21.22 | | |
| pCL-SE-0338.APT73.74 | Yarrowia | APT73.74 |
| pCL-SE-0338.APT73.77 | Yarrowia | APT73.77 |
| pCL-SE-0757 | Yarrowia | APT73.77-F17-GPS1.1 |
| pCL-SE-0776 | Yarrowia | APT73.77-F18-GPS1.1 |
| pCL-SE-0777 | Yarrowia | GPS1.1-F16-APT73.77 |
| pCL-SE-0778 | Yarrowia | GPS1.1-F18-APT73.77 |

TABLE 17
Yarrowia strains

| Strain | Key genes expressed |
|---|---|
| sCL-SE-0128 | HMGR, GPS1.1 |
| SB-00311 | HCS2, PKS1.1, PKC1.1 |

### Screening in Yarrowia

Overnight YPD (10 g/L yeast extract, 20 g/L peptone, 2% dextrose) cultures were inoculated from glycerol stocks of the appropriate strain and grown at 30° C. with 250 rpm shaking. Once cultures had reached an OD600 of 4-6, cultures were centrifuged at 500×g for 5 min, supernatants were discarded, and cell pellets were resuspended in equal volume of water. Resuspended cells were centrifuged at 500×g for 5 min, supernatants were discarded, and cells were resuspended in a volume (75 μL×OD×V_{culture}) of transformation cocktail (45% PEG-400, 0.1 M LiAc, 0.1 M DTT, and 25 ug/100 μL SS Salmon Sperm DNA). For each transformation, >1 μg of plasmid DNA was added to 55 μL cells/transformation cocktail and vortexed for 2 s. Transformations were incubated at 39° C. for 1 h with 250 rpm shaking. Transformations were resuspended in 750 μL YPD with 1 M sorbitol and recovered at 30° C. overnight with 250 rpm shaking. The next day, transformations were centrifuged at 500×g for 5 min, supernatants were discarded, and cell pellets were resuspended in 750 YPD. Resuspended transformations were plated on YPD with appropriate selection or YNBD (6.71 g/L yeast nitrogen base+nitrogen, 0.5% casamino acids, 2% dextrose) agar plates and grown at 30° C. for 2 days. Individual colonies were patched onto YPD plates with appropriate selection or YNBD plates and grown at 30° C. overnight. Patches were used to inoculate 0.5 mL YPD with appropriate selection or YNBD precultures in 96 w blocks and grown at 30° C. for 24-48 h with 1000 rpm shaking. For assays, 0.5 mL YPD with appropriate selection or YNBD cultures containing substrate were inoculated with 2 μL from precultures and grown at 30° C. for 2-4 days with 1000 rpm shaking with 2% glucose added every 24 h. Assay cultures were quenched by addition of 0.5 mL ethanol with 0.2% formic acid and 0.5 mg/mL pentyl-benzoic acid. Precipitates were pelleted by centrifuging at 4600×g for 10 min and then 200 μL was transferred to fresh plates, sealed, and analyzed via HPLC.

### Analytical Methods

All samples after quenching with equal volume of EtOH were centrifuged and were analyzed by HPLC-MS

All samples were quenched with equal volume of EtOH containing 0.2 mg/mL internal standard (3,5-Diisopropyl-2-hydroxybenzoic acid CAS #2215-21-6) centrifuged, and clarified solutions were analyzed by HPLC-MS

### Method A

Column: 2.1×50 mm COSMOCORE PBr (Nacalai USA, Inc.)
Mobile Phase: A; 0.1% formic acid in water, B; 0.1% formic acid in acetonitrile
Flow: 0.45 mL/min
Temp: % 50 Celsius
Gradient: 20%B at 0 min, 70%B at 2.3 min, 89% B at 4.2 min, 20% B at 4.3 min, 20%B at 6 min

TABLE 18
Detection: UV DAD and QToF MS

| Compound | Retention Time (min) |
|---|---|
| Butyric acid | 0.60 |
| Hexanoic acid | 1.53 |
| DOL (Divarinol) | 1.65 |
| DVA (Divarinic acid) | 1.89 |
| OL (Olivetol) | 2.32 |
| OA (Olivetolic acid) | 2.43 |
| Internal Std. | 3.09 |
| CBGVA | 3.57 |
| CBGA | 3.89 |
| F-CBGVA | 4.27 |
| F-CBGA | 4.58 |

All compounds were confirmed based on authentic standards, by retention time, UV profile and MS comparisons. For FCBGA and FCBGA no authentic standards were available so these compounds were identified based on UV profile, and MS analysis (molecular ion and fragmentation pattern)

### Modeling and Mutagenesis

As described in all engineering projects in this work, prior to any mutagenesis approach, structural models of the proteins were created. For this, a variety of commercial and free software packages are available that were used to make structure models using crystal structures of homologous proteins as templates. The selection of the template structures used in the homology modelling process considered three important factors: i) sequence identity between the template enzyme(s) and the target enzyme(s) [only those with >30% sequence identity were used]; ii) the atomic resolution at which the template enzyme(s) were solved; and iii) The percent of sequence coverage between the target enzyme and the template enzyme(s) (i.e., differences in the length of the enzymes). Using this approach 8 to 10 templates were used to generate the homology models. The homology models were evaluated for accuracy using specific software (MolProbity) and if necessary, further refinement and correction of the structure models was achieved using secondary software. Refinement of models entailed rotamer optimization and then the use of GROMACS and energy minimization. Specifically, the top model from multi-template-based modelling was placed in a cubic box with edges 2 nm from any part of the protein being modelled. Periodic boundary conditions were defined, the system was solvated (TIP5P water model; current updated version; gold standard for MD), and the charge of the system neutralized with Na2+ or C12− contingent on the protein and overall charge of the system. Models were then refined using the amber99sb-ildn force-field (widely used force-field for MD), and the simulation was conducted until the potential energy of the entire system converged. The energy minimized PDB was extracted without the neutralizing ions and explicit water molecules, and then subjected to quality improvement using MolProbity. In all cases, refinement improved the overall quality of the initial model significantly.

Finally, the appropriate substrates were docked in the active site using a AutoDock Vina software package and iterative changes to the grid search size. The top two (of a number of possible orientations) docking poses for substrates were selected based on calculated binding energy and the orientation in the active site that brings substrates at the right position for reaction. After this modeling exercise was completed, amino acids in the active site that are 5 Å from each substrate were identified and were selected for mutagenesis.

### Process Development for Making Cannabinoids Through Fermentation

The above CBGA synthases can be used in cell free reactions (in vitro) to produce CBGA and analogs by the feeding of the appropriate substrates or can be introduced into a recombinant organism (yeast, bacteria, fungus, algae, or plant) to improve the flux towards CBGA or any of its analogs. These recombinant organisms will contain the optimized genes described herein to synthesize olivetolic acid and CBGA (or their analogs) engineered mevalonate or MEP pathway to increase flux towards GPP or FPP. To improve flux and increase the intracellular concentration of GPP, mutant farnesyl pyrophosphate synthases may be used as have been described in yeast (Jian G-Z, et al Metabolic Engineering, 2017, 41, 57) or GPP specific synthases can be introduced (Schmidt A, Gershenzon J. Phytochemistry, 2008, 69, 49). Other enzymes in the mevalonate pathway (for example HMG-CoA reductase) may need to be manipulated (truncated or mutated) or be overexpressed. The formation of GPP/FPP and OA can occur when the organism is grown with simple carbon sources, such as glucose, sucrose, glycerol, or another simple or complex sugar mixture. External organic acids with carbon chains varying from 4 to more than 12 (in straight or branched chains) can also be supplemented during growth. With supplementation, introduction of the appropriate acid-CoA synthase may be required to produce the corresponding organic acid-CoAs that can then be used by PKS and PKC to produce OA analogs. The organism can also express the appropriate synthase that cyclizes CBGA or any of its analogs to other cannabinoids as shown in FIG. 2.

SEQUENCES

MPT1 (SEQ ID NO: 1)
MGLSSVCTFSFQTNYHTLLNPHNNNPKTSLLCYRHPKTPIKYSYNNFPSK
HCSTKSFHLQNKCSESLSIAKNSIRAATTNQTEPPESDNHSVATKILNFGKACWKLQRPYT
IIAFTSCACGLFGKELLHNTNLISWSLMFKAFFFLVAVLCIASFTTTINQIYDLHIDRINKPD
LPLASGEISVNTAWIMSIIVALFGLIITIKMKGGPLYIFGYCFGIFGGIVYSVPPFRWKQNPS
TAFLLNFLAHIITNFTFYYASRAALGLPFELRPSFTFLLAFMKSMGSALALIKDASDVEGD
TKFGISTLASKYGSRNLTLFCSGIVLLSYVAAILAGIIWPQAFNSNVMLLSHAILAFWLILQ
TRDFALTNYDPEAGRRFYEFMWKLYYAEYLVYVFI
MPT4 (SEQ ID NO: 2)
MSDNSIATKILNFGHTCWKLQRPYVVKGMISIACGLFGRELFNNRHLFSW
GLMWKAFFALVPILSFNFFAAIMNQIYDVDIDRINKPDLPLVSGEMSIETAWILSIIVALTG
LIVTIKLKSAPLFVFIYIFGIFAGFAYSVPPIRWKQYPFTNFLITISSHVGLAFTSYSATTSAL
GLPFVWRPAFSFIIAFMTVMGMTIAFAKDISDIEGDAKYGVSTVATKLGARNMTFVVSG
VLLLNYLVSISIGIIWPQVFKSNIMILSHAILAFCLIFQTRELALANYASAPSRQFFEFIWLL
YYAEYFVYVFI
MPT4.1 (point mutant) (SEQ ID NO: 52)
MSDNSIATKILNFGHTCWKLQRPYAVKGMISIACGLFGRELFNNRHLFSW
GLMWKAFFALVPILSFNFFAAIMNQIYDVDIDRINKPDLPLVSGEMSIETAWILSIIVALTG
LIVTIKLKSAPLFVFIYIFGIFAGFAYSVPPIRWKQYPFTNFLITISSHVGLAFTSYSATTSAL
GLPFVWRPAFSFIIAFMTVMGMTIAFAKDISDIEGDAKYGVSTVATKLGARNMTFVVSG
VLLLNYLVSISIGIIWPQVFKSNIMILSHAILAFCLIFQTRELALANYASAPSRQFFEFIWLL
YYAEYFVYVFI
MPT21 (SEQ ID NO: 22)
MSDNSIATKILNFGHTCWKLQRPYVVKGMISIACGLFGKELLHNTNLISW
GLMWKAFFALVPILSFNFFAAIMNQIYDVDIDRINKPDLPLVSGEMSIETAWILSIIVALTG
LIVTIKLKSAPLFVFIYIFGIFAGFAYSVPPIRWKQNPSTNFLITISSHVGLAFTSYYASRAAL
GLPFELRPSFTFLLAFMTVMGMTIAFAKDISDIEGDAKYGVSTVATKLGARNMTFVVSG
VLLLNYVAAILAGIIWPQAFNSNVMLLSHAILAFCLIFQTRELALANYASAPSRQFFEFIW
LLYYAEYFVYVFI
MPT26 (SEQ ID NO: 23)
MSDNSIATKILNFGHTCWKLQRPYVVKGMISIACGLFGKELLHNTNLISW
GLMWKAFFALVPILSFNFFAAIMNQIYDVDIDRINKPDLPLVSGEMSIETAWILSIIVALTG
LIVTIKLKSAPLFVFIYIFGIFAGFAYSVPPIRWKQNPSTNFLITISSHVGLAFTSYSATTSAL
GLPFVWRPAFSFIIAFMTVMGMTIAFAKDISDIEGDAKYGVSTVATKLGARNMTFVVSG
VLLLNYVAAILAGIIWPQAFNSNVMLLSHAILAFWLILQTRDFALTNYDPEAGRRFFEFIW
LLYYAEYFVYVFI
MPT31 (SEQ ID NO: 24)
MSDNSIATKILNFGHACWKLQRPYVVKGMISIACGLFGRELLHNTNLISW
GLMWKAFFALVPILSFNFFAAIMNQIYDLHIDRINKPDLPLASGEISVNTAWIMSIIVALTG
LIVTIKLKSAPLFVFIYIFGIFAGFAYSVPPIRWKQNPSTNFLITISSHVGLAFTSYYASRAAL
GLPFELRPSFTFLLAFMTVMGMTIAFAKDISDIEGDAKYGVSTVATKLGARNMTFVVSG
VLLLNYLVSISIGIIWPQVFKSNIMILSHAILAFWLILQTRDFALTNYDPEAGRRFFEFIWLL
YYAEYLVYVFI
APT73.74 (SEQ ID NO: 37)
MDEVYAAVEQTSRLLDVPCSPDRFEPVWKAFGDQLPDSHLVFSMAAGE
AHRGELDFDFSLRPEGADPYTTALEHGFIEPTDHPVGSVLAEVGKRFAIASYGVEYGVVG
GFKKSYAFFPLDDFPPLAQFAEVPSVPPCLAGHVETLTRLGFDDKVSIIGVNYRKNTLNV
YLAASAVDTGDKLALLRAFGYPEPDARVRQFIERSFRLYPTFNWDSSAAERICFAVHTQQ
PGELPAPHDEPTEAFARQVPHVYEGGREFVSGVALAPSGASYYKLAALYQKARRCLH
APT73.77 (SEQ ID NO: 38)
MDEVYAAVEQTSRLLDVPCSPDRFEPVWKAFGDQLPDSHLVFSMAAGE
AHRGELDFDFSLRPEGADPYTTALEHGFIEPTDHPVGSVLAEVGKRFAIASYGVEYGVVG
GFKKSYAFFPLDDFPPLAQFAEVPSVPPCLAGHVETLTRLGFDDKVSIIGVNYRKNTLNV
YLAASAVDTGDKLALLRAFGYPEPDARVRQFIERSFRLYPTFNWDSSAAERICFAVHTQQ
PGELPAPHDEPTEAFARQVPHVYEGGREFVSGVALAPSGASYYKLAALYQKARRCLD
APT89.38 (SEQ ID NO: 39)
MDEVYAAVERTSRLLDVPCSPDRFEPVWKAFGDQLPDSHLVFSMAAGE
AHRGELDFDFSLRPEGADPYTTALEHGFIEPTDHPVGSVLAEVNKRCEIASYGVEYGVVG
GFKKSYAFFPLDDFPPLAEFARIPSVPPCLAGHVDTLTRLGLDDKVSAIGVNYRKNTLNV
YLAASAVATDDKLALLRAFGYPEPDARVRQFIERSFRLYPTFNWDSSAAERICFAVHTQQ
PGELPAPHDEPTEAFAREVPHVYEGGREFVSGVALAPSGAAYYKLAAEYQKERRCL
GPS1.1-F16-APT73.74 (SEQ ID NO: 59)
MSKAKFESVFPRISEELVQLLRDEGLPQDAVQWFSDSLQYNCVGGKLNR
GLSVVDTYQLLTGKKELDDEEYYRLALLGWLIELLQAFWLVSDDIMDESKTRRGQPCW
YLKPKVGMIAIWDAFMLESGIYILLKKHFRQEKYYIDLVELFHDISFKTELGQLVDLLTAP
EDEVDLNRFSLDKHSFIVRYKTAYYSFYLPVVLAMYVAGITNPKDLQQAMDVLIPLGEY
FQVQDDYLDNFGDPEFIGKIGTDIQDNKCSWLVNKALQKATPEQRQILEDNYGVKDKSK
ELVIKKLYDDMKIEQDYLDYEEEVVGDIKKKIEQVDESRGFKKEVLNAFLAKIYKRQKG
GGSGGGSGGGSGGGGSMDEVYAAVEQTSRLLDVPCSPDRFEPVWKAFGDQLPDSHLVF
SMAAGEAHRGELDFDFSLRPEGADPYTTALEHGFIEPTDHPVGSVLAEVGKRFAIASYGV
EYGVVGGFKKSYAFFPLDDFPPLAQFAEVPSVPPCLAGHVETLTRLGFDDKVSIIGVNYR
KNTLNVYLAASAVDTGDKLALLRAFGYPEPDARVRQFIERSFRLYPTFNWDSSAAERICF
AVHTQQPGELPAPHDEPTEAFARQVPHVYEGGREFVSGVALAPSGASYYKLAALYQKA
RRCLH
APT73.74-F17-GPS1.1 (SEQ ID NO: 60)
MDEVYAAVEQTSRLLDVPCSPDRFEPVWKAFGDQLPDSHLVFSMAAGE
AHRGELDFDFSLRPEGADPYTTALEHGFIEPTDHPVGSVLAEVGKRFAIASYGVEYGVVG
GFKKSYAFFPLDDFPPLAQFAEVPSVPPCLAGHVETLTRLGFDDKVSIIGVNYRKNTLNV
YLAASAVDTGDKLALLRAFGYPEPDARVRQFIERSFRLYPTFNWDSSAAERICFAVHTQQ
PGELPAPHDEPTEAFARQVPHVYEGGREFVSGVALAPSGASYYKLAALYQKARRCLHG
GGGSMSKAKFESVFPRISEELVQLLRDEGLPQDAVQWFSDSLQYNCVGGKLNRGLSVVD
TYQLLTGKKELDDEEYYRLALLGWLIELLQAFWLVSDDIMDESKTRRGQPCWYLKPKV
GMIAIWDAFMLESGIYILLKKHFRQEKYYIDLVELFHDISFKTELGQLVDLLTAPEDEVDL
NRFSLDKHSFIVRYKTAYYSFYLPVVLAMYVAGITNPKDLQQAMDVLIPLGEYFQVQDD
YLDNFGDPEFIGKIGTDIQDNKCSWLVNKALQKATPEQRQILEDNYGVKDKSKELVIKKL
YDDMKIEQDYLDYEEEVVGDIKKKIEQVDESRGFKKEVLNAFLAKIYKRQK
GPS1.1-F18-APT73.74 (SEQ ID NO: 61)
MSKAKFESVFPRISEELVQLLRDEGLPQDAVQWFSDSLQYNCVGGKLNR
GLSVVDTYQLLTGKKELDDEEYYRLALLGWLIELLQAFWLVSDDIMDESKTRRGQPCW
YLKPKVGMIAIWDAFMLESGIYILLKKHFRQEKYYIDLVELFHDISFKTELGQLVDLLTAP
EDEVDLNRFSLDKHSFIVRYKTAYYSFYLPVVLAMYVAGITNPKDLQQAMDVLIPLGEY
FQVQDDYLDNFGDPEFIGKIGTDIQDNKCSWLVNKALQKATPEQRQILEDNYGVKDKSK
ELVIKKLYDDMKIEQDYLDYEEEVVGDIKKKIEQVDESRGFKKEVLNAFLAKIYKRQKG
GGGSLEDPAVWEAGKVVAKGVGTADITATTSNGLIASSEEADNAATSMDEVYAAVEQT
SRLLDVPCSPDRFEPVWKAFGDQLPDSHLVFSMAAGEAHRGELDFDFSLRPEGADPYTT
ALEHGFIEPTDHPVGSVLAEVGKRFAIASYGVEYGVVGGFKKSYAFFPLDDFPPLAQFAE
VPSVPPCLAGHVETLTRLGFDDKVSIIGVNYRKNTLNVYLAASAVDTGDKLALLRAFGY
PEPDARVRQFIERSFRLYPTFNWDSSAAERICFAVHTQQPGELPAPHDEPTEAFARQVPHV
YEGGREFVSGVALAPSGASYYKLAALYQKARRCLH
APT73.74-F18-GPS1.1 (SEQ ID NO: 62)
MDEVYAAVEQTSRLLDVPCSPDRFEPVWKAFGDQLPDSHLVFSMAAGE
AHRGELDFDFSLRPEGADPYTTALEHGFIEPTDHPVGSVLAEVGKRFAIASYGVEYGVVG
GFKKSYAFFPLDDFPPLAQFAEVPSVPPCLAGHVETLTRLGFDDKVSIIGVNYRKNTLNV
YLAASAVDTGDKLALLRAFGYPEPDARVRQFIERSFRLYPTFNWDSSAAERICFAVHTQQ
PGELPAPHDEPTEAFARQVPHVYEGGREFVSGVALAPSGASYYKLAALYQKARRCLHG
GGGSLEDPAVWEAGKVVAKGVGTADITATTSNGLIASSEEADNAATSMSKAKFESVFPR
ISEELVQLLRDEGLPQDAVQWFSDSLQYNCVGGKLNRGLSVVDTYQLLTGKKELDDEE
YYRLALLGWLIELLQAFWLVSDDIMDESKTRRGQPCWYLKPKVGMIAIWDAFMLESGIY
ILLKKHFRQEKYYIDLVELFHDISFKTELGQLVDLLTAPEDEVDLNRFSLDKHSFIVRYKT
AYYSFYLPVVLAMYVAGITNPKDLQQAMDVLIPLGEYFQVQDDYLDNFGDPEFIGKIGT
DIQDNKCSWLVNKALQKATPEQRQILEDNYGVKDKSKELVIKKLYDDMKIEQDYLDYE
EEVVGDIKKKIEQVDESRGFKKEVLNAFLAKIYKRQK
GPS1.1-F16-APT73.77 (SEQ ID NO: 63)
MSKAKFESVFPRISEELVQLLRDEGLPQDAVQWFSDSLQYNCVGGKLNR
GLSVVDTYQLLTGKKELDDEEYYRLALLGWLIELLQAFWLVSDDIMDESKTRRGQPCW
YLKPKVGMIAIWDAFMLESGIYILLKKHFRQEKYYIDLVELFHDISFKTELGQLVDLLTAP
EDEVDLNRFSLDKHSFIVRYKTAYYSFYLPVVLAMYVAGITNPKDLQQAMDVLIPLGEY
FQVQDDYLDNFGDPEFIGKIGTDIQDNKCSWLVNKALQKATPEQRQILEDNYGVKDKSK
ELVIKKLYDDMKIEQDYLDYEEEVVGDIKKKIEQVDESRGFKKEVLNAFLAKIYKRQKG
GGSGGGSGGGSGGGGSMDEVYAAVEQTSRLLDVPCSPDRFEPVWKAFGDQLPDSHLVF
SMAAGEAHRGELDFDFSLRPEGADPYTTALEHGFIEPTDHPVGSVLAEVGKRFAIASYGV
EYGVVGGFKKSYAFFPLDDFPPLAQFAEVPSVPPCLAGHVETLTRLGFDDKVSIIGVNYR
KNTLNVYLAASAVDTGDKLALLRAFGYPEPDARVRQFIERSFRLYPTFNWDSSAAERICF
AVHTQQPGELPAPHDEPTEAFARQVPHVYEGGREFVSGVALAPSGASYYKLAALYQKA
RRCLD
APT73.77-F17-GPS1.1 (SEQ ID NO: 64)
MDEVYAAVEQTSRLLDVPCSPDRFEPVWKAFGDQLPDSHLVFSMAAGE
AHRGELDFDFSLRPEGADPYTTALEHGFIEPTDHPVGSVLAEVGKRFAIASYGVEYGVVG
GFKKSYAFFPLDDFPPLAQFAEVPSVPPCLAGHVETLTRLGFDDKVSIIGVNYRKNTLNV
YLAASAVDTGDKLALLRAFGYPEPDARVRQFIERSFRLYPTFNWDSSAAERICFAVHTQQ
PGELPAPHDEPTEAFARQVPHVYEGGREFVSGVALAPSGASYYKLAALYQKARRCLDG
GGGSMSKAKFESVFPRISEELVQLLRDEGLPQDAVQWFSDSLQYNCVGGKLNRGLSVVD
TYQLLTGKKELDDEEYYRLALLGWLIELLQAFWLVSDDIMDESKTRRGQPCWYLKPKV
GMIAIWDAFMLESGIYILLKKHFRQEKYYIDLVELFHDISFKTELGQLVDLLTAPEDEVDL
NRFSLDKHSFIVRYKTAYYSFYLPVVLAMYVAGITNPKDLQQAMDVLIPLGEYFQVQDD
YLDNFGDPEFIGKIGTDIQDNKCSWLVNKALQKATPEQRQILEDNYGVKDKSKELVIKKL
YDDMKIEQDYLDYEEEVVGDIKKKIEQVDESRGFKKEVLNAFLAKIYKRQK
GPS1.1-F18-APT73.77 (SEQ ID NO: 65)
MSKAKFESVFPRISEELVQLLRDEGLPQDAVQWFSDSLQYNCVGGKLNR
GLSVVDTYQLLTGKKELDDEEYYRLALLGWLIELLQAFWLVSDDIMDESKTRRGQPCW
YLKPKVGMIAIWDAFMLESGIYILLKKHFRQEKYYIDLVELFHDISFKTELGQLVDLLTAP
EDEVDLNRFSLDKHSFIVRYKTAYYSFYLPVVLAMYVAGITNPKDLQQAMDVLIPLGEY
FQVQDDYLDNFGDPEFIGKIGTDIQDNKCSWLVNKALQKATPEQRQILEDNYGVKDKSK
ELVIKKLYDDMKIEQDYLDYEEEVVGDIKKKIEQVDESRGFKKEVLNAFLAKIYKRQKG
GGGSLEDPAVWEAGKVVAKGVGTADITATTSNGLIASSEEADNAATSMDEVYAAVEQT
SRLLDVPCSPDRFEPVWKAFGDQLPDSHLVFSMAAGEAHRGELDFDFSLRPEGADPYTT
ALEHGFIEPTDHPVGSVLAEVGKRFAIASYGVEYGVVGGFKKSYAFFPLDDFPPLAQFAE
VPSVPPCLAGHVETLTRLGFDDKVSIIGVNYRKNTLNVYLAASAVDTGDKLALLRAFGY
PEPDARVRQFIERSFRLYPTFNWDSSAAERICFAVHTQQPGELPAPHDEPTEAFARQVPHV
YEGGREFVSGVALAPSGASYYKLAALYQKARRCLD
APT73.77-F18-GPS1.1 (SEQ ID NO: 66)
MDEVYAAVEQTSRLLDVPCSPDRFEPVWKAFGDQLPDSHLVFSMAAGE
AHRGELDFDFSLRPEGADPYTTALEHGFIEPTDHPVGSVLAEVGKRFAIASYGVEYGVVG
GFKKSYAFFPLDDFPPLAQFAEVPSVPPCLAGHVETLTRLGFDDKVSIIGVNYRKNTLNV
YLAASAVDTGDKLALLRAFGYPEPDARVRQFIERSFRLYPTFNWDSSAAERICFAVHTQQ
PGELPAPHDEPTEAFARQVPHVYEGGREFVSGVALAPSGASYYKLAALYQKARRCLDG
GGGSLEDPAVWEAGKVVAKGVGTADITATTSNGLIASSEEADNAATSMSKAKFESVFPR
ISEELVQLLRDEGLPQDAVQWFSDSLQYNCVGGKLNRGLSVVDTYQLLTGKKELDDEE
YYRLALLGWLIELLQAFWLVSDDIMDESKTRRGQPCWYLKPKVGMIAIWDAFMLESGIY
ILLKKHFRQEKYYIDLVELFHDISFKTELGQLVDLLTAPEDEVDLNRFSLDKHSFIVRYKT
AYYSFYLPVVLAMYVAGITNPKDLQQAMDVLIPLGEYFQVQDDYLDNFGDPEFIGKIGT
DIQDNKCSWLVNKALQKATPEQRQILEDNYGVKDKSKELVIKKLYDDMKIEQDYLDYE
EEVVGDIKKKIEQVDESRGFKKEVLNAFLAKIYKRQK
GPS1.1-F16-APT89.38 (SEQ ID NO: 67)
MSKAKFESVFPRISEELVQLLRDEGLPQDAVQWFSDSLQYNCVGGKLNR
GLSVVDTYQLLTGKKELDDEEYYRLALLGWLIELLQAFWLVSDDIMDESKTRRGQPCW
YLKPKVGMIAIWDAFMLESGIYILLKKHFRQEKYYIDLVELFHDISFKTELGQLVDLLTAP
EDEVDLNRFSLDKHSFIVRYKTAYYSFYLPVVLAMYVAGITNPKDLQQAMDVLIPLGEY
FQVQDDYLDNFGDPEFIGKIGTDIQDNKCSWLVNKALQKATPEQRQILEDNYGVKDKSK
ELVIKKLYDDMKIEQDYLDYEEEVVGDIKKKIEQVDESRGFKKEVLNAFLAKIYKRQKG
GGSGGGSGGGSGGGGSMDEVYAAVERTSRLLDVPCSPDRFEPVWKAFGDQLPDSHLVF
SMAAGEAHRGELDFDFSLRPEGADPYTTALEHGFIEPTDHPVGSVLAEVNKRCEIASYGV
EYGVVGGFKKSYAFFPLDDFPPLAEFARIPSVPPCLAGHVDTLTRLGLDDKVSAIGVNYR
KNTLNVYLAASAVATDDKLALLRAFGYPEPDARVRQFIERSFRLYPTFNWDSSAAERICF
AVHTQQPGELPAPHDEPTEAFAREVPHVYEGGREFVSGVALAPSGAAYYKLAAEYQKE
RRCL
APT89.38-F17-GPS1.1 (SEQ ID NO: 68)
MDEVYAAVERTSRLLDVPCSPDRFEPVWKAFGDQLPDSHLVFSMAAGE
AHRGELDFDFSLRPEGADPYTTALEHGFIEPTDHPVGSVLAEVNKRCEIASYGVEYGVVG
GFKKSYAFFPLDDFPPLAEFARIPSVPPCLAGHVDTLTRLGLDDKVSAIGVNYRKNTLNV
YLAASAVATDDKLALLRAFGYPEPDARVRQFIERSFRLYPTFNWDSSAAERICFAVHTQQ
PGELPAPHDEPTEAFAREVPHVYEGGREFVSGVALAPSGAAYYKLAAEYQKERRCLGGG
GSMSKAKFESVFPRISEELVQLLRDEGLPQDAVQWFSDSLQYNCVGGKLNRGLSVVDTY
QLLTGKKELDDEEYYRLALLGWLIELLQAFWLVSDDIMDESKTRRGQPCWYLKPKVGM
IAIWDAFMLESGIYILLKKHFRQEKYYIDLVELFHDISFKTELGQLVDLLTAPEDEVDLNR
FSLDKHSFIVRYKTAYYSFYLPVVLAMYVAGITNPKDLQQAMDVLIPLGEYFQVQDDYL
DNFGDPEFIGKIGTDIQDNKCSWLVNKALQKATPEQRQILEDNYGVKDKSKELVIKKLY
DDMKIEQDYLDYEEEVVGDIKKKIEQVDESRGFKKEVLNAFLAKIYKRQK
GPS1.1-F18-APT89.38 (SEQ ID NO: 69)
MSKAKFESVFPRISEELVQLLRDEGLPQDAVQWFSDSLQYNCVGGKLNR
GLSVVDTYQLLTGKKELDDEEYYRLALLGWLIELLQAFWLVSDDIMDESKTRRGQPCW
YLKPKVGMIAIWDAFMLESGIYILLKKHFRQEKYYIDLVELFHDISFKTELGQLVDLLTAP
EDEVDLNRFSLDKHSFIVRYKTAYYSFYLPVVLAMYVAGITNPKDLQQAMDVLIPLGEY
FQVQDDYLDNFGDPEFIGKIGTDIQDNKCSWLVNKALQKATPEQRQILEDNYGVKDKSK
ELVIKKLYDDMKIEQDYLDYEEEVVGDIKKKIEQVDESRGFKKEVLNAFLAKIYKRQKG
GGGSLEDPAVWEAGKVVAKGVGTADITATTSNGLIASSEEADNAATSMDEVYAAVERT
SRLLDVPCSPDRFEPVWKAFGDQLPDSHLVFSMAAGEAHRGELDFDFSLRPEGADPYTT
ALEHGFIEPTDHPVGSVLAEVNKRCEIASYGVEYGVVGGFKKSYAFFPLDDFPPLAEFARI
PSVPPCLAGHVDTLTRLGLDDKVSAIGVNYRKNTLNVYLAASAVATDDKLALLRAFGYP
EPDARVRQFIERSFRLYPTFNWDSSAAERICFAVHTQQPGELPAPHDEPTEAFAREVPHV
YEGGREFVSGVALAPSGAAYYKLAAEYQKERRCL
APT89.38-F18-GPS1.1 (SEQ ID NO: 70)
MDEVYAAVERTSRLLDVPCSPDRFEPVWKAFGDQLPDSHLVFSMAAGE
AHRGELDFDFSLRPEGADPYTTALEHGFIEPTDHPVGSVLAEVNKRCEIASYGVEYGVVG
GFKKSYAFFPLDDFPPLAEFARIPSVPPCLAGHVDTLTRLGLDDKVSAIGVNYRKNTLNV
YLAASAVATDDKLALLRAFGYPEPDARVRQFIERSFRLYPTFNWDSSAAERICFAVHTQQ
PGELPAPHDEPTEAFAREVPHVYEGGREFVSGVALAPSGAAYYKLAAEYQKERRCLGGG
GSLEDPAVWEAGKVVAKGVGTADITATTSNGLIASSEEADNAATSMSKAKFESVFPRISE
ELVQLLRDEGLPQDAVQWFSDSLQYNCVGGKLNRGLSVVDTYQLLTGKKELDDEEYYR
LALLGWLIELLQAFWLVSDDIMDESKTRRGQPCWYLKPKVGMIAIWDAFMLESGIYILL
KKHFRQEKYYIDLVELFHDISFKTELGQLVDLLTAPEDEVDLNRFSLDKHSFIVRYKTAY
YSFYLPVVLAMYVAGITNPKDLQQAMDVLIPLGEYFQVQDDYLDNFGDPEFIGKIGTDIQ
DNKCSWLVNKALQKATPEQRQILEDNYGVKDKSKELVIKKLYDDMKIEQDYLDYEEEV
VGDIKKKIEQVDESRGFKKEVLNAFLAKIYKRQK
GPS1.1 (SEQ ID NO: 4)
MSKAKFESVFPRISEELVQLLRDEGLPQDAVQWFSDSLQYNCVGGKLNR
GLSVVDTYQLLTGKKELDDEEYYRLALLGWLIELLQAFWLVSDDIMDESKTRRGQPCW
YLKPKVGMIAIWDAFMLESGIYILLKKHFRQEKYYIDLVELFHDISFKTELGQLVDLLTAP
EDEVDLNRFSLDKHSFIVRYKTAYYSFYLPVVLAMYVAGITNPKDLQQAMDVLIPLGEY
FQVQDDYLDNFGDPEFIGKIGTDIQDNKCSWLVNKALQKATPEQRQILEDNYGVKDKSK
ELVIKKLYDDMKIEQDYLDYEEEVVGDIKKKIEQVDESRGFKKEVLNAFLAKIYKRQK
GPS2 (SEQ ID NO: 5)
MKDVSLSSFDAHDLDLDKFPEVVRDRLTQFLDAQELTIADIGAPVTDAV
AHLRSFVLNGGKRIRPLYAWAGFLAAQGHKNSSEKLESVLDAAASLEFIQACALIHDDII
DSSDTRRGAPTVHRAVEADHRANNFEGDPEHFGVSVSILAGDMALVWAEDMLQDSGLS
AEALARTRDAWRGMRTEVIGGQLLDIYLESHANESVELADSVNRFKTAAYTIARPLHLG
ASIAGGSPQLIDALLHYGHDIGIAFQLRDDLLGVFGDPAITGKPAGDDIREGKRTVLLALA
LQRADKQSPEAATAIRAGVGKVTSPEDIAVITEHIRATGAEEEVEQRISQLTESGLAHLDD
VDIPDEVRAQLRALAIRSTERRM
GPS3 (SEQ ID NO: 6)
MQLLNPPQKGKKAVEFDFNKYMDSKAMTVNEALNKAIPLRYPQKIYES
MRYSLLAGGKRVRPVLCIAACELVGGTEELAIPTACAIEMIHTMSLMHDDLPCIDNDDLR
RGKPTNHKIFGEDTAVTAGNALHSYAFEHIAVSTSKTVGADRILRMVSELGRATGSEGV
MGGQMVDIASEGDPSIDLQTLEWIHIHKTAMLLECSVVCGAIIGGASEIVIERARRYARCV
GLLFQVVDDILDVTKSSDELGKTAGKDLISDKATYPKLMGLEKAKEFSDELLNRAKGEL
SCFDPVKAAPLLGLADYVAFRQN
GPS1.1-F9-MPT4 (SEQ ID NO: 18)
MSKAKFESVFPRISEELVQLLRDEGLPQDAVQWFSDSLQYNCVGGKLNR
GLSVVDTYQLLTGKKELDDEEYYRLALLGWLIELLQAFWLVSDDIMDESKTRRGQPCW
YLKPKVGMIAIWDAFMLESGIYILLKKHFRQEKYYIDLVELFHDISFKTELGQLVDLLTAP
EDEVDLNRFSLDKHSFIVRYKTAYYSFYLPVVLAMYVAGITNPKDLQQAMDVLIPLGEY
FQVQDDYLDNFGDPEFIGKIGTDIQDNKCSWLVNKALQKATPEQRQILEDNYGVKDKSK
ELVIKKLYDDMKIEQDYLDYEEEVVGDIKKKIEQVDESRGFKKEVLNAFLAKIYKRQKG
GGGSGGGGSGGGGSGGGGSGGGGSGGGGSMSDNSIATKILNFGHTCWKLQRPYVVKG
MISIACGLFGRELFNNRHLFSWGLMWKAFFALVPILSFNFFAAIMNQIYDVDIDRINKPDL
PLVSGEMSIETAWILSIIVALTGLIVTIKLKSAPLFVFIYIFGIFAGFAYSVPPIRWKQYPFTN
FLITISSHVGLAFTSYSATTSALGLPFVWRPAFSFIIAFMTVMGMTIAFAKDISDIEGDAKY
GVSTVATKLGARNMTFVVSGVLLLNYLVSISIGIIWPQVFKSNIMILSHAILAFCLIFQTRE
LALANYASAPSRQFFEFIWLLYYAEYFVYVFI
GPS1.1-F5-MPT4 (SEQ ID NO: 17)
MSKAKFESVFPRISEELVQLLRDEGLPQDAVQWFSDSLQYNCVGGKLNR
GLSVVDTYQLLTGKKELDDEEYYRLALLGWLIELLQAFWLVSDDIMDESKTRRGQPCW
YLKPKVGMIAIWDAFMLESGIYILLKKHFRQEKYYIDLVELFHDISFKTELGQLVDLLTAP
EDEVDLNRFSLDKHSFIVRYKTAYYSFYLPVVLAMYVAGITNPKDLQQAMDVLIPLGEY
FQVQDDYLDNFGDPEFIGKIGTDIQDNKCSWLVNKALQKATPEQRQILEDNYGVKDKSK
ELVIKKLYDDMKIEQDYLDYEEEVVGDIKKKIEQVDESRGFKKEVLNAFLAKIYKRQKG
GGGSGGGGSGGGGSMSDNSIATKILNFGHTCWKLQRPYVVKGMISIACGLFGRELFNNR
HLFSWGLMWKAFFALVPILSFNFFAAIMNQIYDVDIDRINKPDLPLVSGEMSIETAWILSII
VALTGLIVTIKLKSAPLFVFIYIFGIFAGFAYSVPPIRWKQYPFTNFLITISSHVGLAFTSYSA
TTSALGLPFVWRPAFSFIIAFMTVMGMTIAFAKDISDIEGDAKYGVSTVATKLGARNMTF
VVSGVLLLNYLVSISIGIIWPQVFKSNIMILSHAILAFCLIFQTRELALANYASAPSRQFFEFI
WLLYYAEYFVYVFI
GPS1.1-F10-MPT4 (SEQ ID NO: 19)
MSKAKFESVFPRISEELVQLLRDEGLPQDAVQWFSDSLQYNCVGGKLNR
GLSVVDTYQLLTGKKELDDEEYYRLALLGWLIELLQAFWLVSDDIMDESKTRRGQPCW
YLKPKVGMIAIWDAFMLESGIYILLKKHFRQEKYYIDLVELFHDISFKTELGQLVDLLTAP
EDEVDLNRFSLDKHSFIVRYKTAYYSFYLPVVLAMYVAGITNPKDLQQAMDVLIPLGEY
FQVQDDYLDNFGDPEFIGKIGTDIQDNKCSWLVNKALQKATPEQRQILEDNYGVKDKSK
ELVIKKLYDDMKIEQDYLDYEEEVVGDIKKKIEQVDESRGFKKEVLNAFLAKIYKRQKG
GAEAAAKEAAAKAAEAAAKEAAAKAAEAAAKEAAAKAAEAAAKEAAAKAGGMSDN
SIATKILNFGHTCWKLQRPYVVKGMISIACGLFGRELFNNRHLFSWGLMWKAFFALVPIL
SFNFFAAIMNQIYDVDIDRINKPDLPLVSGEMSIETAWILSIIVALTGLIVTIKLKSAPLFVFI
YIFGIFAGFAYSVPPIRWKQYPFTNFLITISSHVGLAFTSYSATTSALGLPFVWRPAFSFIIAF
MTVMGMTIAFAKDISDIEGDAKYGVSTVATKLGARNMTFVVSGVLLLNYLVSISIGIIWP
QVFKSNIMILSHAILAFCLIFQTRELALANYASAPSRQFFEFIWLLYYAEYFVYVFI
GPS1.1-F11-MPT4 (SEQ ID NO: 16)
MSKAKFESVFPRISEELVQLLRDEGLPQDAVQWFSDSLQYNCVGGKLNR
GLSVVDTYQLLTGKKELDDEEYYRLALLGWLIELLQAFWLVSDDIMDESKTRRGQPCW
YLKPKVGMIAIWDAFMLESGIYILLKKHFRQEKYYIDLVELFHDISFKTELGQLVDLLTAP
EDEVDLNRFSLDKHSFIVRYKTAYYSFYLPVVLAMYVAGITNPKDLQQAMDVLIPLGEY
FQVQDDYLDNFGDPEFIGKIGTDIQDNKCSWLVNKALQKATPEQRQILEDNYGVKDKSK
ELVIKKLYDDMKIEQDYLDYEEEVVGDIKKKIEQVDESRGFKKEVLNAFLAKIYKRQKG
GAEAAAKEAAAKAGGSGGGSGGGGSGGSGGGGSGGGGSMSDNSIATKILNFGHTCWK
LQRPYVVKGMISIACGLFGRELFNNRHLFSWGLMWKAFFALVPILSFNFFAAIMNQIYDV
DIDRINKPDLPLVSGEMSIETAWILSIIVALTGLIVTIKLKSAPLFVFIYIFGIFAGFAYSVPPI
RWKQYPFTNFLITISSHVGLAFTSYSATTSALGLPFVWRPAFSFIIAFMTVMGMTIAFAKDI
SDIEGDAKYGVSTVATKLGARNMTFVVSGVLLLNYLVSISIGIIWPQVFKSNIMILSHAIL
AFCLIFQTRELALANYASAPSRQFFEFIWLLYYAEYFVYVFI
GPS3-F11-MPT4 (SEQ ID NO: 20)
MQLLNPPQKGKKAVEFDFNKYMDSKAMTVNEALNKAIPLRYPQKIYES
MRYSLLAGGKRVRPVLCIAACELVGGTEELAIPTACAIEMIHTMSLMHDDLPCIDNDDLR
RGKPTNHKIFGEDTAVTAGNALHSYAFEHIAVSTSKTVGADRILRMVSELGRATGSEGV
MGGQMVDIASEGDPSIDLQTLEWIHIHKTAMLLECSVVCGAIIGGASEIVIERARRYARCV
GLLFQVVDDILDVTKSSDELGKTAGKDLISDKATYPKLMGLEKAKEFSDELLNRAKGEL
SCFDPVKAAPLLGLADYVAFRQNGGAEAAAKEAAAKAGGSGGGSGGGGSGGSGGGGS
GGGGSMSDNSIATKILNFGHTCWKLQRPYVVKGMISIACGLFGRELFNNRHLFSWGLM
WKAFFALVPILSFNFFAAIMNQIYDVDIDRINKPDLPLVSGEMSIETAWILSIIVALTGLIVT
IKLKSAPLFVFIYIFGIFAGFAYSVPPIRWKQYPFTNFLITISSHVGLAFTSYSATTSALGLPF
VWRPAFSFIIAFMTVMGMTIAFAKDISDIEGDAKYGVSTVATKLGARNMTFVVSGVLLL
NYLVSISIGIIWPQVFKSNIMILSHAILAFCLIFQTRELALANYASAPSRQFFEFIWLLYYAE
YFVYVFI
GPS2-F11-MPT4 (SEQ ID NO: 21)
MKDVSLSSFDAHDLDLDKFPEVVRDRLTQFLDAQELTIADIGAPVTDAV
AHLRSFVLNGGKRIRPLYAWAGFLAAQGHKNSSEKLESVLDAAASLEFIQACALIHDDII
DSSDTRRGAPTVHRAVEADHRANNFEGDPEHFGVSVSILAGDMALVWAEDMLQDSGLS
AEALARTRDAWRGMRTEVIGGQLLDIYLESHANESVELADSVNRFKTAAYTIARPLHLG
ASIAGGSPQLIDALLHYGHDIGIAFQLRDDLLGVFGDPAITGKPAGDDIREGKRTVLLALA
LQRADKQSPEAATAIRAGVGKVTSPEDIAVITEHIRATGAEEEVEQRISQLTESGLAHLDD
VDIPDEVRAQLRALAIRSTERRMGGAEAAAKEAAAKAGGSGGGSGGGGSGGSGGGGSG
GGGSMSDNSIATKILNFGHTCWKLQRPYVVKGMISIACGLFGRELFNNRHLFSWGLMW
KAFFALVPILSFNFFAAIMNQIYDVDIDRINKPDLPLVSGEMSIETAWILSIIVALTGLIVTIK
LKSAPLFVFIYIFGIFAGFAYSVPPIRWKQYPFTNFLITISSHVGLAFTSYSATTSALGLPFV
WRPAFSFIIAFMTVMGMTIAFAKDISDIEGDAKYGVSTVATKLGARNMTFVVSGVLLLN
YLVSISIGIIWPQVFKSNIMILSHAILAFCLIFQTRELALANYASAPSRQFFEFIWLLYYAEY
FVYVFI
GPS1.1-F9-MPT4.1 (SEQ ID NO: 53)
MSKAKFESVFPRISEELVQLLRDEGLPQDAVQWFSDSLQYNCVGGKLNR
GLSVVDTYQLLTGKKELDDEEYYRLALLGWLIELLQAFWLVSDDIMDESKTRRGQPCW
YLKPKVGMIAIWDAFMLESGIYILLKKHFRQEKYYIDLVELFHDISFKTELGQLVDLLTAP
EDEVDLNRFSLDKHSFIVRYKTAYYSFYLPVVLAMYVAGITNPKDLQQAMDVLIPLGEY
FQVQDDYLDNFGDPEFIGKIGTDIQDNKCSWLVNKALQKATPEQRQILEDNYGVKDKSK
ELVIKKLYDDMKIEQDYLDYEEEVVGDIKKKIEQVDESRGFKKEVLNAFLAKIYKRQKG
GGGSGGGGSGGGGSGGGGSGGGGSGGGGSMSDNSIATKILNFGHTCWKLQRPYVVKG
MISIACGLFGRELFNNRHLFSWGLMWKAFFALVPILSFNFFAAIMNQIYDVDIDRINKPDL
PLVSGEMSIETAWILSIIVALTGLIVTIKLKSAPLFVFIYIFGIFAGFAYSVPPIRWKQYPFTN
FLITISSHVGLAFTSYSATTSALGLPFVWRPAFSFIIAFMTVMGMTIAFAKDISDIEGDAKY
GVSTVATKLGARNMTFVVSGVLLLNYLVSISIGIIWPQVFKSNIMILSHAILAFCLIFQTRE
LALANYASAPSRQFFEFIWLLYYAEYFVYVFI
GPS1.1-F5-MPT4.1 (SEQ ID NO: 54)
MSKAKFESVFPRISEELVQLLRDEGLPQDAVQWFSDSLQYNCVGGKLNR
GLSVVDTYQLLTGKKELDDEEYYRLALLGWLIELLQAFWLVSDDIMDESKTRRGQPCW
YLKPKVGMIAIWDAFMLESGIYILLKKHFRQEKYYIDLVELFHDISFKTELGQLVDLLTAP
EDEVDLNRFSLDKHSFIVRYKTAYYSFYLPVVLAMYVAGITNPKDLQQAMDVLIPLGEY
FQVQDDYLDNFGDPEFIGKIGTDIQDNKCSWLVNKALQKATPEQRQILEDNYGVKDKSK
ELVIKKLYDDMKIEQDYLDYEEEVVGDIKKKIEQVDESRGFKKEVLNAFLAKIYKRQKG
GGGSGGGGSGGGGSMSDNSIATKILNFGHTCWKLQRPYAVKGMISIACGLFGRELFNNR
HLFSWGLMWKAFFALVPILSFNFFAAIMNQIYDVDIDRINKPDLPLVSGEMSIETAWILSII
VALTGLIVTIKLKSAPLFVFIYIFGIFAGFAYSVPPIRWKQYPFTNFLITISSHVGLAFTSYSA
TTSALGLPFVWRPAFSFIIAFMTVMGMTIAFAKDISDIEGDAKYGVSTVATKLGARNMTF
VVSGVLLLNYLVSISIGIIWPQVFKSNIMILSHAILAFCLIFQTRELALANYASAPSRQFFEFI
WLLYYAEYFVYVFI
GPS1.1-F10-MPT4.1 (SEQ ID NO: 55)
MSKAKFESVFPRISEELVQLLRDEGLPQDAVQWFSDSLQYNCVGGKLNR
GLSVVDTYQLLTGKKELDDEEYYRLALLGWLIELLQAFWLVSDDIMDESKTRRGQPCW
YLKPKVGMIAIWDAFMLESGIYILLKKHFRQEKYYIDLVELFHDISFKTELGQLVDLLTAP
EDEVDLNRFSLDKHSFIVRYKTAYYSFYLPVVLAMYVAGITNPKDLQQAMDVLIPLGEY
FQVQDDYLDNFGDPEFIGKIGTDIQDNKCSWLVNKALQKATPEQRQILEDNYGVKDKSK
ELVIKKLYDDMKIEQDYLDYEEEVVGDIKKKIEQVDESRGFKKEVLNAFLAKIYKRQKG
GAEAAAKEAAAKAAEAAAKEAAAKAAEAAAKEAAAKAAEAAAKEAAAKAGGMSDN
SIATKILNFGHTCWKLQRPYAVKGMISIACGLFGRELFNNRHLFSWGLMWKAFFALVPIL
SFNFFAAIMNQIYDVDIDRINKPDLPLVSGEMSIETAWILSIIVALTGLIVTIKLKSAPLFVFI
YIFGIFAGFAYSVPPIRWKQYPFTNFLITISSHVGLAFTSYSATTSALGLPFVWRPAFSFIIAF
MTVMGMTIAFAKDISDIEGDAKYGVSTVATKLGARNMTFVVSGVLLLNYLVSISIGIIWP
QVFKSNIMILSHAILAFCLIFQTRELALANYASAPSRQFFEFIWLLYYAEYFVYVFI
GPS1.1-F11-MPT4.1 (SEQ ID NO: 56)
MSKAKFESVFPRISEELVQLLRDEGLPQDAVQWFSDSLQYNCVGGKLNR
GLSVVDTYQLLTGKKELDDEEYYRLALLGWLIELLQAFWLVSDDIMDESKTRRGQPCW
YLKPKVGMIAIWDAFMLESGIYILLKKHFRQEKYYIDLVELFHDISFKTELGQLVDLLTAP
EDEVDLNRFSLDKHSFIVRYKTAYYSFYLPVVLAMYVAGITNPKDLQQAMDVLIPLGEY
FQVQDDYLDNFGDPEFIGKIGTDIQDNKCSWLVNKALQKATPEQRQILEDNYGVKDKSK
ELVIKKLYDDMKIEQDYLDYEEEVVGDIKKKIEQVDESRGFKKEVLNAFLAKIYKRQKG
GAEAAAKEAAAKAGGSGGGSGGGGSGGSGGGGSGGGGSMSDNSIATKILNFGHTCWK
LQRPYAVKGMISIACGLFGRELFNNRHLFSWGLMWKAFFALVPILSFNFFAAIMNQIYDV
DIDRINKPDLPLVSGEMSIETAWILSIIVALTGLIVTIKLKSAPLFVFIYIFGIFAGFAYSVPPI
RWKQYPFTNFLITISSHVGLAFTSYSATTSALGLPFVWRPAFSFIIAFMTVMGMTIAFAKDI
SDIEGDAKYGVSTVATKLGARNMTFVVSGVLLLNYLVSISIGIIWPQVFKSNIMILSHAIL
AFCLIFQTRELALANYASAPSRQFFEFIWLLYYAEYFVYVFI
GPS3-F11-MPT4.1 (SEQ ID NO: 57)
MQLLNPPQKGKKAVEFDFNKYMDSKAMTVNEALNKAIPLRYPQKIYESMRYSLLAGGK
RVRPVLCIAACELVGGTEELAIPTACAIEMIHTMSLMHDDLPCIDNDDLRRGKPTNHKIFG
EDTAVTAGNALHSYAFEHIAVSTSKTVGADRILRMVSELGRATGSEGVMGGQMVDIASE
GDPSIDLQTLEWIHIHKTAMLLECSVVCGAIIGGASEIVIERARRYARCVGLLFQVVDDIL
DVTKSSDELGKTAGKDLISDKATYPKLMGLEKAKEFSDELLNRAKGELSCFDPVKAAPL
LGLADYVAFRQNGGAEAAAKEAAAKAGGSGGGSGGGGSGGSGGGGSGGGGSMSDNSI
ATKILNFGHTCWKLQRPYAVKGMISIACGLFGRELFNNRHLFSWGLMWKAFFALVPILS
FNFFAAIMNQIYDVDIDRINKPDLPLVSGEMSIETAWILSIIVALTGLIVTIKLKSAPLFVFIY
IFGIFAGFAYSVPPIRWKQYPFTNFLITISSHVGLAFTSYSATTSALGLPFVWRPAFSFIIAF
MTVMGMTIAFAKDISDIEGDAKYGVSTVATKLGARNMTFVVSGVLLLNYLVSISIGIIWP
QVFKSNIMILSHAILAFCLIFQTRELALANYASAPSRQFFEFIWLLYYAEYFVYVFI
GPS2-F11-MPT4.1 (SEQ ID NO: 58)
MKDVSLSSFDAHDLDLDKFPEVVRDRLTQFLDAQELTIADIGAPVTDAV
AHLRSFVLNGGKRIRPLYAWAGFLAAQGHKNSSEKLESVLDAAASLEFIQACALIHDDII
DSSDTRRGAPTVHRAVEADHRANNFEGDPEHFGVSVSILAGDMALVWAEDMLQDSGLS
AEALARTRDAWRGMRTEVIGGQLLDIYLESHANESVELADSVNRFKTAAYTIARPLHLG
ASIAGGSPQLIDALLHYGHDIGIAFQLRDDLLGVFGDPAITGKPAGDDIREGKRTVLLALA
LQRADKQSPEAATAIRAGVGKVTSPEDIAVITEHIRATGAEEEVEQRISQLTESGLAHLDD
VDIPDEVRAQLRALAIRSTERRMGGAEAAAKEAAAKAGGSGGGSGGGGSGGSGGGGSG
GGGSMSDNSIATKILNFGHTCWKLQRPYAVKGMISIACGLFGRELFNNRHLFSWGLMW
KAFFALVPILSFNFFAAIMNQIYDVDIDRINKPDLPLVSGEMSIETAWILSIIVALTGLIVTIK
LKSAPLFVFIYIFGIFAGFAYSVPPIRWKQYPFTNFLITISSHVGLAFTSYSATTSALGLPFV
WRPAFSFIIAFMTVMGMTIAFAKDISDIEGDAKYGVSTVATKLGARNMTFVVSGVLLLN
YLVSISIGIIWPQVFKSNIMILSHAILAFCLIFQTRELALANYASAPSRQFFEFIWLLYYAEY
FVYVFI
GPS1.1-F11-MPT21 (SEQ ID NO: 25)
MSKAKFESVFPRISEELVQLLRDEGLPQDAVQWFSDSLQYNCVGGKLNR
GLSVVDTYQLLTGKKELDDEEYYRLALLGWLIELLQAFWLVSDDIMDESKTRRGQPCW
YLKPKVGMIAIWDAFMLESGIYILLKKHFRQEKYYIDLVELFHDISFKTELGQLVDLLTAP
EDEVDLNRFSLDKHSFIVRYKTAYYSFYLPVVLAMYVAGITNPKDLQQAMDVLIPLGEY
FQVQDDYLDNFGDPEFIGKIGTDIQDNKCSWLVNKALQKATPEQRQILEDNYGVKDKSK
ELVIKKLYDDMKIEQDYLDYEEEVVGDIKKKIEQVDESRGFKKEVLNAFLAKIYKRQKG
GAEAAAKEAAAKAGGSGGGSGGGGSGGSGGGGSGGGGSMSDNSIATKILNFGHTCWK
LQRPYVVKGMISIACGLFGKELLHNTNLISWGLMWKAFFALVPILSFNFFAAIMNQIYDV
DIDRINKPDLPLVSGEMSIETAWILSIIVALTGLIVTIKLKSAPLFVFIYIFGIFAGFAYSVPPI
RWKQNPSTNFLITISSHVGLAFTSYYASRAALGLPFELRPSFTFLLAFMTVMGMTIAFAKD
ISDIEGDAKYGVSTVATKLGARNMTFVVSGVLLLNYVAAILAGIIWPQAFNSNVMLLSH
AILAFCLIFQTRELALANYASAPSRQFFEFIWLLYYAEYFVYVFI
GPS1.1-F11-MPT26 (SEQ ID NO: 26)
MSKAKFESVFPRISEELVQLLRDEGLPQDAVQWFSDSLQYNCVGGKLNR
GLSVVDTYQLLTGKKELDDEEYYRLALLGWLIELLQAFWLVSDDIMDESKTRRGQPCW
YLKPKVGMIAIWDAFMLESGIYILLKKHFRQEKYYIDLVELFHDISFKTELGQLVDLLTAP
EDEVDLNRFSLDKHSFIVRYKTAYYSFYLPVVLAMYVAGITNPKDLQQAMDVLIPLGEY
FQVQDDYLDNFGDPEFIGKIGTDIQDNKCSWLVNKALQKATPEQRQILEDNYGVKDKSK
ELVIKKLYDDMKIEQDYLDYEEEVVGDIKKKIEQVDESRGFKKEVLNAFLAKIYKRQKG
GAEAAAKEAAAKAGGSGGGSGGGGSGGSGGGGSGGGGSMSDNSIATKILNFGHTCWK
LQRPYVVKGMISIACGLFGKELLHNTNLISWGLMWKAFFALVPILSFNFFAAIMNQIYDV
DIDRINKPDLPLVSGEMSIETAWILSIIVALTGLIVTIKLKSAPLFVFIYIFGIFAGFAYSVPPI
RWKQNPSTNFLITISSHVGLAFTSYSATTSALGLPFVWRPAFSFIIAFMTVMGMTIAFAKDI
SDIEGDAKYGVSTVATKLGARNMTFVVSGVLLLNYVAAILAGIIWPQAFNSNVMLLSHA
ILAFWLILQTRDFALTNYDPEAGRRFFEFIWLLYYAEYFVYVFI
GPS1.1-F11-MPT31 (SEQ ID NO: 27)
MSKAKFESVFPRISEELVQLLRDEGLPQDAVQWFSDSLQYNCVGGKLNR
GLSVVDTYQLLTGKKELDDEEYYRLALLGWLIELLQAFWLVSDDIMDESKTRRGQPCW
YLKPKVGMIAIWDAFMLESGIYILLKKHFRQEKYYIDLVELFHDISFKTELGQLVDLLTAP
EDEVDLNRFSLDKHSFIVRYKTAYYSFYLPVVLAMYVAGITNPKDLQQAMDVLIPLGEY
FQVQDDYLDNFGDPEFIGKIGTDIQDNKCSWLVNKALQKATPEQRQILEDNYGVKDKSK
ELVIKKLYDDMKIEQDYLDYEEEVVGDIKKKIEQVDESRGFKKEVLNAFLAKIYKRQKG
GAEAAAKEAAAKAGGSGGGSGGGGSGGSGGGGSGGGGSMSDNSIATKILNFGHACWK
LQRPYVVKGMISIACGLFGRELLHNTNLISWGLMWKAFFALVPILSFNFFAAIMNQIYDL
HIDRINKPDLPLASGEISVNTAWIMSIIVALTGLIVTIKLKSAPLFVFIYIFGIFAGFAYSVPPI
RWKQNPSTNFLITISSHVGLAFTSYYASRAALGLPFELRPSFTFLLAFMTVMGMTIAFAKD
ISDIEGDAKYGVSTVATKLGARNMTFVVSGVLLLNYLVSISIGIIWPQVFKSNIMILSHAIL
AFWLILQTRDFALTNYDPEAGRRFFEFIWLLYYAEYLVYVFI
F1 (SEQ ID NO: 7)
GGGGSGGGGSAEAAAKAEAAAKAGGGGSGGGGS
F2 (SEQ ID NO: 8)
GGAEAAAKEAAAKAGGSGGGSGGGGSGGS
F3 (SEQ ID NO: 9)
GGAEAAAKEAAAKAAEAAAKEAAAKAGGGSPGPGPGGGS
F4 (SEQ ID NO: 10)
GSSSSSSGSSSSSSGSSSSSSGSSSSSSGSSSSSSG
F5 (SEQ ID NO: 11)
GGGGSGGGGSGGGGS
F6 (SEQ ID NO: 12)
GGEAAAKEAAAKEAAAKGG
F7 (SEQ ID NO: 13)
GGAEAAAKEAAAKAPAPAPAG
F8 (SEQ ID NO: 14)
GTPTPTPTPTG
F9 (SEQ ID NO: 15)
GGGGSGGGGSGGGGSGGGGSGGGGSGGGGS
F10 (SEQ ID NO: 28)
GGAEAAAKEAAAKAAEAAAKEAAAKAAEAAAKEAAAKAAEAAAKEA
AAKAGG
F11 (SEQ ID NO: 29)
GGAEAAAKEAAAKAGGSGGGSGGGGSGGSGGGGSGGGGS
F12 (SEQ ID NO: 30)
GGGGSGGGGS
F13 (SEQ ID NO: 31)
GGSGSAGSAAGSGEFGG
F14 (SEQ ID NO: 32)
GGAEAAAKEAAAKAPAPAPAEAAAKEAAAKAGG
F15 (SEQ ID NO: 33)
GGSGGAEAAAKEAAAKAGGSGG
F16 (SEQ ID NO: 34)
GGGSGGGSGGGSGGGGS
F17 (SEQ ID NO: 35)
GGGGS
F18 (SEQ ID NO: 36)
GGGGSLEDPAVWEAGKVVAKGVGTADITATTSNGLIASSEEADNAATS
MPT21.1 (SEQ ID NO: 71)
MSDNSIATKILNFGHTCWKLQRPFVVKGMISIACGLFGKELLHNTNLISW
GLMWKAFFALVPILSFNFFAAIMNQIYDVDIDRINKPDLPLVSGEMSIETAWILSIIVALTG
LIVTIKLKSAPLFVFIYIFGIFAGFAYSVPPIRWKQNPSTNFLITISSHVGLAFTSYYASRAAL
GLPFELRPSFTFLLAFMTVMGMTIAFAKDISDIEGDAKYGVSTVATKLGARNMTFVVSG
VLLLNYVAAILAGIIWPQAFNSNVMLLSHAILAFCLIFQTRELALANYASAPSRQFFEFIW
LLYYAEYFVYVFI
MPT21.2 (SEQ ID NO: 72)
MSDNSIATKILNFGHTCWKLQRPMVVKGMISIACGLFGKELLHNTNLISW
GLMWKAFFALVPILSFNFFAAIMNQIYDVDIDRINKPDLPLVSGEMSIETAWILSIIVALTG
LIVTIKLKSAPLFVFIYIFGIFAGFAYSVPPIRWKQNPSTNFLITISSHVGLAFTSYYASRAAL
GLPFELRPSFTFLLAFMTVMGMTIAFAKDISDIEGDAKYGVSTVATKLGARNMTFVVSG
VLLLNYVAAILAGIIWPQAFNSNVMLLSHAILAFCLIFQTRELALANYASAPSRQFFEFIW
LLYYAEYFVYVFI
MPT21.3 (SEQ ID NO: 73)
MSDNSIATKILNFGHTCWKLQRPYAVKGMISIACGLFGKELLHNTNLISW
GLMWKAFFALVPILSFNFFAAIMNQIYDVDIDRINKPDLPLVSGEMSIETAWILSIIVALTG
LIVTIKLKSAPLFVFIYIFGIFAGFAYSVPPIRWKQNPSTNFLITISSHVGLAFTSYYASRAAL
GLPFELRPSFTFLLAFMTVMGMTIAFAKDISDIEGDAKYGVSTVATKLGARNMTFVVSG
VLLLNYVAAILAGIIWPQAFNSNVMLLSHAILAFCLIFQTRELALANYASAPSRQFFEFIW
LLYYAEYFVYVFI
MPT21.4 (SEQ ID NO: 74)
MSDNSIATKILNFGHTCWKLQRPYVVKGAISIACGLFGKELLHNTNLISW
GLMWKAFFALVPILSFNFFAAIMNQIYDVDIDRINKPDLPLVSGEMSIETAWILSIIVALTG
LIVTIKLKSAPLFVFIYIFGIFAGFAYSVPPIRWKQNPSTNFLITISSHVGLAFTSYYASRAAL
GLPFELRPSFTFLLAFMTVMGMTIAFAKDISDIEGDAKYGVSTVATKLGARNMTFVVSG
VLLLNYVAAILAGIIWPQAFNSNVMLLSHAILAFCLIFQTRELALANYASAPSRQFFEFIW
LLYYAEYFVYVFI
MPT21.5 (SEQ ID NO: 75)
MSDNSIATKILNFGHTCWKLQRPYVVKGMITIACGLFGKELLHNTNLISW
GLMWKAFFALVPILSFNFFAAIMNQIYDVDIDRINKPDLPLVSGEMSIETAWILSIIVALTG
LIVTIKLKSAPLFVFIYIFGIFAGFAYSVPPIRWKQNPSTNFLITISSHVGLAFTSYYASRAAL
GLPFELRPSFTFLLAFMTVMGMTIAFAKDISDIEGDAKYGVSTVATKLGARNMTFVVSG
VLLLNYVAAILAGIIWPQAFNSNVMLLSHAILAFCLIFQTRELALANYASAPSRQFFEFIW
LLYYAEYFVYVFI
MPT21.6 (SEQ ID NO: 76)
MSDNSIATKILNFGHTCWKLQRPYVVKGMIVIACGLFGKELLHNTNLISW
GLMWKAFFALVPILSFNFFAAIMNQIYDVDIDRINKPDLPLVSGEMSIETAWILSIIVALTG
LIVTIKLKSAPLFVFIYIFGIFAGFAYSVPPIRWKQNPSTNFLITISSHVGLAFTSYYASRAAL
GLPFELRPSFTFLLAFMTVMGMTIAFAKDISDIEGDAKYGVSTVATKLGARNMTFVVSG
VLLLNYVAAILAGIIWPQAFNSNVMLLSHAILAFCLIFQTRELALANYASAPSRQFFEFIW
LLYYAEYFVYVFI
MPT21.7 (SEQ ID NO: 77)
MSDNSIATKILNFGHTCWKLQRPYVVKGMIAIACGLFGKELLHNTNLISW
GLMWKAFFALVPILSFNFFAAIMNQIYDVDIDRINKPDLPLVSGEMSIETAWILSIIVALTG
LIVTIKLKSAPLFVFIYIFGIFAGFAYSVPPIRWKQNPSTNFLITISSHVGLAFTSYYASRAAL
GLPFELRPSFTFLLAFMTVMGMTIAFAKDISDIEGDAKYGVSTVATKLGARNMTFVVSG
VLLLNYVAAILAGIIWPQAFNSNVMLLSHAILAFCLIFQTRELALANYASAPSRQFFEFIW
LLYYAEYFVYVFI
MPT21.8 (SEQ ID NO: 78)
MSDNSIATKILNFGHTCWKLQRPYVVKGMISIACGLFGKELLHNTNLISW
GLMWKAFFALVPILSFNFFAGIMNQIYDVDIDRINKPDLPLVSGEMSIETAWILSIIVALTG
LIVTIKLKSAPLFVFIYIFGIFAGFAYSVPPIRWKQNPSTNFLITISSHVGLAFTSYYASRAAL
GLPFELRPSFTFLLAFMTVMGMTIAFAKDISDIEGDAKYGVSTVATKLGARNMTFVVSG
VLLLNYVAAILAGIIWPQAFNSNVMLLSHAILAFCLIFQTRELALANYASAPSRQFFEFIW
LLYYAEYFVYVFI
MPT21.9 (SEQ ID NO: 79)
MSDNSIATKILNFGHTCWKLQRPYVVKGMISIACGLFGKELLHNTNLISW
GLMWKAFFALVPILSFNFFASIMNQIYDVDIDRINKPDLPLVSGEMSIETAWILSIIVALTG
LIVTIKLKSAPLFVFIYIFGIFAGFAYSVPPIRWKQNPSTNFLITISSHVGLAFTSYYASRAAL
GLPFELRPSFTFLLAFMTVMGMTIAFAKDISDIEGDAKYGVSTVATKLGARNMTFVVSG
VLLLNYVAAILAGIIWPQAFNSNVMLLSHAILAFCLIFQTRELALANYASAPSRQFFEFIW
LLYYAEYFVYVFI
MPT21.10 (SEQ ID NO: 80)
MSDNSIATKILNFGHTCWKLQRPYVVKGMISIACGLFGKELLHNTNLISW
GLMWKAFFALVPILSFNFFAAIMNQIYDVDMDRINKPDLPLVSGEMSIETAWILSIIVALT
GLIVTIKLKSAPLFVFIYIFGIFAGFAYSVPPIRWKQNPSTNFLITISSHVGLAFTSYYASRAA
LGLPFELRPSFTFLLAFMTVMGMTIAFAKDISDIEGDAKYGVSTVATKLGARNMTFVVSG
VLLLNYVAAILAGIIWPQAFNSNVMLLSHAILAFCLIFQTRELALANYASAPSRQFFEFIW
LLYYAEYFVYVFI
MPT21.11 (SEQ ID NO: 81)
MSDNSIATKILNFGHTCWKLQRPYVVKGMISIACGLFGKELLHNTNLISW
GLMWKAFFALVPILSFNFFAAIMNQIYDVDIDRVNKPDLPLVSGEMSIETAWILSIIVALT
GLIVTIKLKSAPLFVFIYIFGIFAGFAYSVPPIRWKQNPSTNFLITISSHVGLAFTSYYASRAA
LGLPFELRPSFTFLLAFMTVMGMTIAFAKDISDIEGDAKYGVSTVATKLGARNMTFVVSG
VLLLNYVAAILAGIIWPQAFNSNVMLLSHAILAFCLIFQTRELALANYASAPSRQFFEFIW
LLYYAEYFVYVFI
MPT21.12 (SEQ ID NO: 82)
MSDNSIATKILNFGHTCWKLQRPYVVKGMISIACGLFGKELLHNTNLISW
GLMWKAFFALVPILSFNFFAAIMNQIYDVDIDRINKPDLPLVSGEMSIETAWILSIIVALTG
LIVTIKLKSAPLFVFIYIFGIFAGFAYSVPPIRWKQNPSTNFEITISSHVGLAFTSYYASRAAL
GLPFELRPSFTFLLAFMTVMGMTIAFAKDISDIEGDAKYGVSTVATKLGARNMTFVVSG
VLLLNYVAAILAGIIWPQAFNSNVMLLSHAILAFCLIFQTRELALANYASAPSRQFFEFIW
LLYYAEYFVYVFI
MPT21.13 (SEQ ID NO: 83)
MSDNSIATKILNFGHTCWKLQRPYVVKGMISIACGLFGKELLHNTNLISW
GLMWKAFFALVPILSFNFFAAIMNQIYDVDIDRINKPDLPLVSGEMSIETAWILSIIVALTG
LIVTIKLKSAPLFVFIYIFGIFAGFAYSVPPIRWKQNPSTNFLITIASHVGLAFTSYYASRAA
LGLPFELRPSFTFLLAFMTVMGMTIAFAKDISDIEGDAKYGVSTVATKLGARNMTFVVSG
VLLLNYVAAILAGIIWPQAFNSNVMLLSHAILAFCLIFQTRELALANYASAPSRQFFEFIW
LLYYAEYFVYVFI
MPT21.14 (SEQ ID NO: 84)
MSDNSIATKILNFGHTCWKLQRPYVVKGMISIACGLFGKELLHNTNLISW
GLMWKAFFALVPILSFNFFAAIMNQIYDVDIDRINKPDLPLVSGEMSIETAWILSIIVALTG
LIVTIKLKSAPLFVFIYIFGIFAGFAYSVPPIRWKQNPSTNFLITIGSHVGLAFTSYYASRAA
LGLPFELRPSFTFLLAFMTVMGMTIAFAKDISDIEGDAKYGVSTVATKLGARNMTFVVSG
VLLLNYVAAILAGIIWPQAFNSNVMLLSHAILAFCLIFQTRELALANYASAPSRQFFEFIW
LLYYAEYFVYVFI
MPT21.15 (SEQ ID NO: 85)
MSDNSIATKILNFGHTCWKLQRPYVVKGMISIACGLFGKELLHNTNLISW
GLMWKAFFALVPILSFNFFAAIMNQIYDVDIDRINKPDLPLVSGEMSIETAWILSIIVALTG
LIVTIKLKSAPLFVFIYIFGIFAGFAYSVPPIRWKQNPSTNFLITIVSHVGLAFTSYYASRAA
LGLPFELRPSFTFLLAFMTVMGMTIAFAKDISDIEGDAKYGVSTVATKLGARNMTFVVSG
VLLLNYVAAILAGIIWPQAFNSNVMLLSHAILAFCLIFQTRELALANYASAPSRQFFEFIW
LLYYAEYFVYVFI
MPT21.16 (SEQ ID NO: 86)
MSDNSIATKILNFGHTCWKLQRPYVVKGMISIACGLFGKELLHNTNLISW
GLMWKAFFALVPILSFNFFAAIMNQIYDVDIDRINKPDLPLVSGEMSIETAWILSIIVALTG
LIVTIKLKSAPLFVFIYIFGIFAGFAYSVPPIRWKQNPSTNFLITISSHVGLAFTSYYASRAAL
GLPFELRPSFTFLLAFMTVMGMTIGFAKDISDIEGDAKYGVSTVATKLGARNMTFVVSG
VLLLNYVAAILAGIIWPQAFNSNVMLLSHAILAFCLIFQTRELALANYASAPSRQFFEFIW
LLYYAEYFVYVFI
MPT21.17 (SEQ ID NO: 87)
MSDNSIATKILNFGHTCWKLQRPYVVKGMISIACGLFGKELLHNTNLISW
GLMWKAFFALVPILSFNFFAAIMNQIYDVDIDRINKPDLPLVSGEMSIETAWILSIIVALTG
LIVTIKLKSAPLFVFIYIFGIFAGFAYSVPPIRWKQNPSTNFLITISSHVGLAFTSYYASRAAL
GLPFELRPSFTFLLAFMTVMGMTIAFAKDISDIEGDAKYGVSTVATKLGARNMTFVVSG
VLLLNYVAAILAGIIWPQAFNSNVMLLSHAILAFCLIFQQRELALANYASAPSRQFFEFIW
LLYYAEYFVYVFI
MPT21.18 (SEQ ID NO: 88)
MSDNSIATKILNFGHTCWKLQRPYVVKGMISIACGLFGKELLHNTNLISW
GLMWKAFFALVPILSFNFFAAIMNQIYDVDIDRINKPDLPLVSGEMSIETAWILSIIVALTG
LIVTIKLKSAPLFVFIYIFGIFAGFAYSVPPIRWKQNPSTNFLITISSHVGLAFTSYYASRAAL
GLPFELRPSFTFLLAFMTVMGMTIAFAKDISDIEGDAKYGVSTVATKLGARNMTFVVSG
VLLLNYVAAILAGIIWPQAFNSNVMLLSHAILAFCLIFQARELALANYASAPSRQFFEFIW
LLYYAEYFVYVFI
MPT21.19 (SEQ ID NO: 89)
MSDNSIATKILNFGHTCWKLQRPYVVKGMISIACGLFGKELLHNTNLISW
GLMWKAFFALVPILSFNFFAAIMNQIYDVDIDRINKPDLPLVSGEMSIETAWILSIIVALTG
LIVTIKLKSAPLFVFIYIFGIFAGFAYSVPPIRWKQNPSTNFLITISSHVGLAFTSYYASRAAL
GLPFELRPSFTFLLAFMTVMGMTIAFAKDISDIEGDAKYGVSTVATKLGARNMTFVVSG
VLLLNYVAAILAGIIWPQAFNSNVMLLSHAILAFCLIFQTRELALANYAAAPSRQFFEFIW
LLYYAEYFVYVFI
MPT21.20 (SEQ ID NO: 90)
MSDNSIATKILNFGHTCWKLQRPYVVKGMISIACGLFGKELLHNTNLISW
GLMWKAFFALVPILSFNFFAAIMNQIYDVDIDRINKPDLPLVSGEMSIETAWILSIIVALTG
LIVTIKLKSAPLFVFIYIFGIFAGFAYSVPPIRWKQNPSTNFLITISSHVGLAFTSYYASRAAL
GLPFELRPSFTFLLAFMTVMGMTIAFAKDISDIEGDAKYGVSTVATKLGARNMTFVVSG
VLLLNYVAAILAGIIWPQAFNSNVMLLSHAILAFCLIFQTRELALANYAGAPSRQFFEFIW
LLYYAEYFVYVFI
MPT21.22 (SEQ ID NO: 91)
MSDNSIATKILNFGHTCWKLQRPYVVKGMISIACGLFGKELLHNTNLISW
GLMWKAFFALVPILSFNFFAAIMNQIYDVDIDRINKPDLPLVSGEMSIETAWILSIIVALTG
LIVTIKLKSAPLFVFIYIFGIFAGFAYSVPPIRWKQNPSTNFLITISSHVGLAFTSYYASRAAL
GLPFELRPSFTFLLAFMTVMGMTIAFAKDISDIEGDAKYGVSTVATKLGARNMTFVVSG
VLLLNYVAAILAGIIWPQAFNSNVMLLSHAILAFCLIFQTRELALANYASAPSRQFFEFLW
LLYYAEYFVYVFI
GPS1.1-F11-PKC1.1-F11-MPT4 (SEQ ID NO: 92)
MSKAKFESVFPRISEELVQLLRDEGLPQDAVQWFSDSLQYNCVGGKLNR
GLSVVDTYQLLTGKKELDDEEYYRLALLGWLIELLQAFWLVSDDIMDESKTRRGQPCW
YLKPKVGMIAIWDAFMLESGIYILLKKHFRQEKYYIDLVELFHDISFKTELGQLVDLLTAP
EDEVDLNRFSLDKHSFIVRYKTAYYSFYLPVVLAMYVAGITNPKDLQQAMDVLIPLGEY
FQVQDDYLDNFGDPEFIGKIGTDIQDNKCSWLVNKALQKATPEQRQILEDNYGVKDKSK
ELVIKKLYDDMKIEQDYLDYEEEVVGDIKKKIEQVDESRGFKKEVLNAFLAKIYKRQKG
GAEAAAKEAAAKAGGSGGGSGGGGSGGSGGGGSGGGGSMAVKHLIVLKFKDEITEAQ
KEEFFKTFVNLVNIIPAMKDVYWGKDVTQKNKEEGYTHIVEVTFESVETIQDYIIHPAHV
GFGDVYRSFWEKLLIFDYTPRKGGAEAAAKEAAAKAGGSGGGSGGGGSGGSGGGGSG
GGGSMSDNSIATKILNFGHTCWKLQRPYVVKGMISIACGLFGRELFNNRHLFSWGLMW
KAFFALVPILSFNFFAAIMNQIYDVDIDRINKPDLPLVSGEMSIETAWILSIIVALTGLIVTIK
LKSAPLFVFIYIFGIFAGFAYSVPPIRWKQYPFTNFLITISSHVGLAFTSYSATTSALGLPFV
WRPAFSFIIAFMTVMGMTIAFAKDISDIEGDAKYGVSTVATKLGARNMTFVVSGVLLLN
YLVSISIGIIWPQVFKSNIMILSHAILAFCLIFQTRELALANYASAPSRQFFEFIWLLYYAEY
FVYVFI
GPS1.1-F11-PKC1.1-F11-MPT4.1 (SEQ ID NO: 93)
MSKAKFESVFPRISEELVQLLRDEGLPQDAVQWFSDSLQYNCVGGKLNR
GLSVVDTYQLLTGKKELDDEEYYRLALLGWLIELLQAFWLVSDDIMDESKTRRGQPCW
YLKPKVGMIAIWDAFMLESGIYILLKKHFRQEKYYIDLVELFHDISFKTELGQLVDLLTAP
EDEVDLNRFSLDKHSFIVRYKTAYYSFYLPVVLAMYVAGITNPKDLQQAMDVLIPLGEY
FQVQDDYLDNFGDPEFIGKIGTDIQDNKCSWLVNKALQKATPEQRQILEDNYGVKDKSK
ELVIKKLYDDMKIEQDYLDYEEEVVGDIKKKIEQVDESRGFKKEVLNAFLAKIYKRQKG
GAEAAAKEAAAKAGGSGGGSGGGGSGGSGGGGSGGGGSMAVKHLIVLKFKDEITEAQ
KEEFFKTFVNLVNIIPAMKDVYWGKDVTQKNKEEGYTHIVEVTFESVETIQDYIIHPAHV
GFGDVYRSFWEKLLIFDYTPRKGGAEAAAKEAAAKAGGSGGGSGGGGSGGSGGGGSG
GGGSMSDNSIATKILNFGHTCWKLQRPYAVKGMISIACGLFGRELFNNRHLFSWGLMW
KAFFALVPILSFNFFAAIMNQIYDVDIDRINKPDLPLVSGEMSIETAWILSIIVALTGLIVTIK
LKSAPLFVFIYIFGIFAGFAYSVPPIRWKQYPFTNFLITISSHVGLAFTSYSATTSALGLPFV
WRPAFSFIIAFMTVMGMTIAFAKDISDIEGDAKYGVSTVATKLGARNMTFVVSGVLLLN
YLVSISIGIIWPQVFKSNIMILSHAILAFCLIFQTRELALANYASAPSRQFFEFIWLLYYAEY
FVYVFI
GPS1.1-F11-PKC1.1-F11-MPT21.3 (SEQ ID NO: 94)
MSKAKFESVFPRISEELVQLLRDEGLPQDAVQWFSDSLQYNCVGGKLNR
GLSVVDTYQLLTGKKELDDEEYYRLALLGWLIELLQAFWLVSDDIMDESKTRRGQPCW
YLKPKVGMIAIWDAFMLESGIYILLKKHFRQEKYYIDLVELFHDISFKTELGQLVDLLTAP
EDEVDLNRFSLDKHSFIVRYKTAYYSFYLPVVLAMYVAGITNPKDLQQAMDVLIPLGEY
FQVQDDYLDNFGDPEFIGKIGTDIQDNKCSWLVNKALQKATPEQRQILEDNYGVKDKSK
ELVIKKLYDDMKIEQDYLDYEEEVVGDIKKKIEQVDESRGFKKEVLNAFLAKIYKRQKG
GAEAAAKEAAAKAGGSGGGSGGGGSGGSGGGGSGGGGSMAVKHLIVLKFKDEITEAQ
KEEFFKTFVNLVNIIPAMKDVYWGKDVTQKNKEEGYTHIVEVTFESVETIQDYIIHPAHV
GFGDVYRSFWEKLLIFDYTPRKGGAEAAAKEAAAKAGGSGGGSGGGGSGGSGGGGSG
GGGSMSDNSIATKILNFGHTCWKLQRPYAVKGMISIACGLFGKELLHNTNLISWGLMWK
AFFALVPILSFNFFAAIMNQIYDVDIDRINKPDLPLVSGEMSIETAWILSIIVALTGLIVTIKL
KSAPLFVFIYIFGIFAGFAYSVPPIRWKQNPSTNFLITISSHVGLAFTSYYASRAALGLPFEL
RPSFTFLLAFMTVMGMTIAFAKDISDIEGDAKYGVSTVATKLGARNMTFVVSGVLLLNY
VAAILAGIIWPQAFNSNVMLLSHAILAFCLIFQTRELALANYASAPSRQFFEFIWLLYYAE
YFVYVFI
GPS1.1-F11-PKC4.33-F11-MPT4.1 (SEQ ID NO: 95)
MSKAKFESVFPRISEELVQLLRDEGLPQDAVQWFSDSLQYNCVGGKLNR
GLSVVDTYQLLTGKKELDDEEYYRLALLGWLIELLQAFWLVSDDIMDESKTRRGQPCW
YLKPKVGMIAIWDAFMLESGIYILLKKHFRQEKYYIDLVELFHDISFKTELGQLVDLLTAP
EDEVDLNRFSLDKHSFIVRYKTAYYSFYLPVVLAMYVAGITNPKDLQQAMDVLIPLGEY
FQVQDDYLDNFGDPEFIGKIGTDIQDNKCSWLVNKALQKATPEQRQILEDNYGVKDKSK
ELVIKKLYDDMKIEQDYLDYEEEVVGDIKKKIEQVDESRGFKKEVLNAFLAKIYKRQKG
GAEAAAKEAAAKAGGSGGGSGGGGSGGSGGGGSGGGGSMGEANKGVVKHVIILKFKE
GITEAQKEEMFKTYVNLVNLVPAMKAVQWGKLEVNNKLGNGGYTHIVESTFESVETIQ
DYIIHPAHVGFGDVYRSFWEKLLIFDYTPTIVLPNSSYGGAEAAAKEAAAKAGGSGGGSG
GGGSGGSGGGGSGGGGSMSDNSIATKILNFGHTCWKLQRPYAVKGMISIACGLFGRELF
NNRHLFSWGLMWKAFFALVPILSFNFFAAIMNQIYDVDIDRINKPDLPLVSGEMSIETAWI
LSIIVALTGLIVTIKLKSAPLFVFIYIFGIFAGFAYSVPPIRWKQYPFTNFLITISSHVGLAFTS
YSATTSALGLPFVWRPAFSFIIAFMTVMGMTIAFAKDISDIEGDAKYGVSTVATKLGARN
MTFVVSGVLLLNYLVSISIGIIWPQVFKSNIMILSHAILAFCLIFQTRELALANYASAPSRQF
FEFIWLLYYAEYFVYVFI
GPS1.1-F11-PKC4.33-F11-MPT21.3 (SEQ ID NO: 96)
MSKAKFESVFPRISEELVQLLRDEGLPQDAVQWFSDSLQYNCVGGKLNR
GLSVVDTYQLLTGKKELDDEEYYRLALLGWLIELLQAFWLVSDDIMDESKTRRGQPCW
YLKPKVGMIAIWDAFMLESGIYILLKKHFRQEKYYIDLVELFHDISFKTELGQLVDLLTAP
EDEVDLNRFSLDKHSFIVRYKTAYYSFYLPVVLAMYVAGITNPKDLQQAMDVLIPLGEY
FQVQDDYLDNFGDPEFIGKIGTDIQDNKCSWLVNKALQKATPEQRQILEDNYGVKDKSK
ELVIKKLYDDMKIEQDYLDYEEEVVGDIKKKIEQVDESRGFKKEVLNAFLAKIYKRQKG
GAEAAAKEAAAKAGGSGGGSGGGGSGGSGGGGSGGGGSMGEANKGVVKHVIILKFKE
GITEAQKEEMFKTYVNLVNLVPAMKAVQWGKLEVNNKLGNGGYTHIVESTFESVETIQ
DYIIHPAHVGFGDVYRSFWEKLLIFDYTPTIVLPNSSYGGAEAAAKEAAAKAGGSGGGSG
GGGSGGSGGGGSGGGGSMSDNSIATKILNFGHTCWKLQRPYAVKGMISIACGLFGKELL
HNTNLISWGLMWKAFFALVPILSFNFFAAIMNQIYDVDIDRINKPDLPLVSGEMSIETAWI
LSIIVALTGLIVTIKLKSAPLFVFIYIFGIFAGFAYSVPPIRWKQNPSTNFLITISSHVGLAFTS
YYASRAALGLPFELRPSFTFLLAFMTVMGMTIAFAKDISDIEGDAKYGVSTVATKLGARN
MTFVVSGVLLLNYVAAILAGIIWPQAFNSNVMLLSHAILAFCLIFQTRELALANYASAPSR
QFFEFIWLLYYAEYFVYVFI
GPS1.1-PKC4.33-MPT4.1 (SEQ ID NO: 97)
MSKAKFESVFPRISEELVQLLRDEGLPQDAVQWFSDSLQYNCVGGKLNR
GLSVVDTYQLLTGKKELDDEEYYRLALLGWLIELLQAFWLVSDDIMDESKTRRGQPCW
YLKPKVGMIAIWDAFMLESGIYILLKKHFRQEKYYIDLVELFHDISFKTELGQLVDLLTAP
EDEVDLNRFSLDKHSFIVRYKTAYYSFYLPVVLAMYVAGITNPKDLQQAMDVLIPLGEY
FQVQDDYLDNFGDPEFIGKIGTDIQDNKCSWLVNKALQKATPEQRQILEDNYGVKDKSK
ELVIKKLYDDMKIEQDYLDYEEEVVGDIKKKIEQVDESRGFKKEVLNAFLAKIYKRQKM
GEANKGVVKHVIILKFKEGITEAQKEEMFKTYVNLVNLVPAMKAVQWGKLEVNNKLG
NGGYTHIVESTFESVETIQDYIIHPAHVGFGDVYRSFWEKLLIFDYTPTIVLPNSSYMSDNS
IATKILNFGHTCWKLQRPYAVKGMISIACGLFGRELFNNRHLFSWGLMWKAFFALVPILS
FNFFAAIMNQIYDVDIDRINKPDLPLVSGEMSIETAWILSIIVALTGLIVTIKLKSAPLFVFIY
IFGIFAGFAYSVPPIRWKQYPFTNFLITISSHVGLAFTSYSATTSALGLPFVWRPAFSFIIAF
MTVMGMTIAFAKDISDIEGDAKYGVSTVATKLGARNMTFVVSGVLLLNYLVSISIGIIWP
QVFKSNIMILSHAILAFCLIFQTRELALANYASAPSRQFFEFIWLLYYAEYFVYVFI
GPS1.1-PKC4.33-MPT21.3 (SEQ ID NO: 98)
MSKAKFESVFPRISEELVQLLRDEGLPQDAVQWFSDSLQYNCVGGKLNR
GLSVVDTYQLLTGKKELDDEEYYRLALLGWLIELLQAFWLVSDDIMDESKTRRGQPCW
YLKPKVGMIAIWDAFMLESGIYILLKKHFRQEKYYIDLVELFHDISFKTELGQLVDLLTAP
EDEVDLNRFSLDKHSFIVRYKTAYYSFYLPVVLAMYVAGITNPKDLQQAMDVLIPLGEY
FQVQDDYLDNFGDPEFIGKIGTDIQDNKCSWLVNKALQKATPEQRQILEDNYGVKDKSK
ELVIKKLYDDMKIEQDYLDYEEEVVGDIKKKIEQVDESRGFKKEVLNAFLAKIYKRQKM
GEANKGVVKHVIILKFKEGITEAQKEEMFKTYVNLVNLVPAMKAVQWGKLEVNNKLG
NGGYTHIVESTFESVETIQDYIIHPAHVGFGDVYRSFWEKLLIFDYTPTIVLPNSSYMSDNS
IATKILNFGHTCWKLQRPYAVKGMISIACGLFGKELLHNTNLISWGLMWKAFFALVPILS
FNFFAAIMNQIYDVDIDRINKPDLPLVSGEMSIETAWILSIIVALTGLIVTIKLKSAPLFVFIY
IFGIFAGFAYSVPPIRWKQNPSTNFLITISSHVGLAFTSYYASRAALGLPFELRPSFTFLLAF
MTVMGMTIAFAKDISDIEGDAKYGVSTVATKLGARNMTFVVSGVLLLNYVAAILAGIIW
PQAFNSNVMLLSHAILAFCLIFQTRELALANYASAPSRQFFEFIWLLYYAEYFVYVFI
PKC1.1-F11-GPS1.1-F11-MPT4.1 (SEQ ID NO: 99)
MAVKHLIVLKFKDEITEAQKEEFFKTFVNLVNIIPAMKDVYWGKDVTQK
NKEEGYTHIVEVTFESVETIQDYIIHPAHVGFGDVYRSFWEKLLIFDYTPRKGGAEAAAK
EAAAKAGGSGGGSGGGGSGGSGGGGSGGGGSMSKAKFESVFPRISEELVQLLRDEGLPQ
DAVQWFSDSLQYNCVGGKLNRGLSVVDTYQLLTGKKELDDEEYYRLALLGWLIELLQA
FWLVSDDIMDESKTRRGQPCWYLKPKVGMIAIWDAFMLESGIYILLKKHFRQEKYYIDL
VELFHDISFKTELGQLVDLLTAPEDEVDLNRFSLDKHSFIVRYKTAYYSFYLPVVLAMYV
AGITNPKDLQQAMDVLIPLGEYFQVQDDYLDNFGDPEFIGKIGTDIQDNKCSWLVNKAL
QKATPEQRQILEDNYGVKDKSKELVIKKLYDDMKIEQDYLDYEEEVVGDIKKKIEQVDE
SRGFKKEVLNAFLAKIYKRQKGGAEAAAKEAAAKAGGSGGGSGGGGSGGSGGGGSGG
GGSMSDNSIATKILNFGHTCWKLQRPYAVKGMISIACGLFGRELFNNRHLFSWGLMWK
AFFALVPILSFNFFAAIMNQIYDVDIDRINKPDLPLVSGEMSIETAWILSIIVALTGLIVTIKL
KSAPLFVFIYIFGIFAGFAYSVPPIRWKQYPFTNFLITISSHVGLAFTSYSATTSALGLPFVW
RPAFSFIIAFMTVMGMTIAFAKDISDIEGDAKYGVSTVATKLGARNMTFVVSGVLLLNYL
VSISIGIIWPQVFKSNIMILSHAILAFCLIFQTRELALANYASAPSRQFFEFIWLLYYAEYFV
YVFI
PKC4.33-F11-GPS1.1-F11-MPT4.1 (SEQ ID NO: 100)
MGEANKGVVKHVIILKFKEGITEAQKEEMFKTYVNLVNLVPAMKAVQW
GKLEVNNKLGNGGYTHIVESTFESVETIQDYIIHPAHVGFGDVYRSFWEKLLIFDYTPTIV
LPNSSYGGAEAAAKEAAAKAGGSGGGSGGGGSGGSGGGGSGGGGSMSKAKFESVFPRI
SEELVQLLRDEGLPQDAVQWFSDSLQYNCVGGKLNRGLSVVDTYQLLTGKKELDDEEY
YRLALLGWLIELLQAFWLVSDDIMDESKTRRGQPCWYLKPKVGMIAIWDAFMLESGIYI
LLKKHFRQEKYYIDLVELFHDISFKTELGQLVDLLTAPEDEVDLNRFSLDKHSFIVRYKTA
YYSFYLPVVLAMYVAGITNPKDLQQAMDVLIPLGEYFQVQDDYLDNFGDPEFIGKIGTDI
QDNKCSWLVNKALQKATPEQRQILEDNYGVKDKSKELVIKKLYDDMKIEQDYLDYEEE
VVGDIKKKIEQVDESRGFKKEVLNAFLAKIYKRQKGGAEAAAKEAAAKAGGSGGGSGG
GGSGGSGGGGSGGGGSMSDNSIATKILNFGHTCWKLQRPYAVKGMISIACGLFGRELFN
NRHLFSWGLMWKAFFALVPILSFNFFAAIMNQIYDVDIDRINKPDLPLVSGEMSIETAWIL
SIIVALTGLIVTIKLKSAPLFVFIYIFGIFAGFAYSVPPIRWKQYPFTNFLITISSHVGLAFTSY
SATTSALGLPFVWRPAFSFIIAFMTVMGMTIAFAKDISDIEGDAKYGVSTVATKLGARNM
TFVVSGVLLLNYLVSISIGIIWPQVFKSNIMILSHAILAFCLIFQTRELALANYASAPSRQFF
EFIWLLYYAEYFVYVFI
PKC4.33-GPS1.1-F11-MPT4.1 (SEQ ID NO: 101)
MGEANKGVVKHVIILKFKEGITEAQKEEMFKTYVNLVNLVPAMKAVQW
GKLEVNNKLGNGGYTHIVESTFESVETIQDYIIHPAHVGFGDVYRSFWEKLLIFDYTPTIV
LPNSSYMSKAKFESVFPRISEELVQLLRDEGLPQDAVQWFSDSLQYNCVGGKLNRGLSV
VDTYQLLTGKKELDDEEYYRLALLGWLIELLQAFWLVSDDIMDESKTRRGQPCWYLKP
KVGMIAIWDAFMLESGIYILLKKHFRQEKYYIDLVELFHDISFKTELGQLVDLLTAPEDEV
DLNRFSLDKHSFIVRYKTAYYSFYLPVVLAMYVAGITNPKDLQQAMDVLIPLGEYFQVQ
DDYLDNFGDPEFIGKIGTDIQDNKCSWLVNKALQKATPEQRQILEDNYGVKDKSKELVI
KKLYDDMKIEQDYLDYEEEVVGDIKKKIEQVDESRGFKKEVLNAFLAKIYKRQKGGAE
AAAKEAAAKAGGSGGGSGGGGSGGSGGGGSGGGGSMSDNSIATKILNFGHTCWKLQRP
YAVKGMISIACGLFGRELFNNRHLFSWGLMWKAFFALVPILSFNFFAAIMNQIYDVDIDR
INKPDLPLVSGEMSIETAWILSIIVALTGLIVTIKLKSAPLFVFIYIFGIFAGFAYSVPPIRWK
QYPFTNFLITISSHVGLAFTSYSATTSALGLPFVWRPAFSFIIAFMTVMGMTIAFAKDISDIE
GDAKYGVSTVATKLGARNMTFVVSGVLLLNYLVSISIGIIWPQVFKSNIMILSHAILAFCLI
FQTRELALANYASAPSRQFFEFIWLLYYAEYFVYVFI
PKC1.1-F11-GPS1.1-F11-MPT21.3 (SEQ ID NO: 102)
MAVKHLIVLKFKDEITEAQKEEFFKTFVNLVNIIPAMKDVYWGKDVTQK
NKEEGYTHIVEVTFESVETIQDYIIHPAHVGFGDVYRSFWEKLLIFDYTPRKGGAEAAAK
EAAAKAGGSGGGSGGGGSGGSGGGGSGGGGSMSKAKFESVFPRISEELVQLLRDEGLPQ
DAVQWFSDSLQYNCVGGKLNRGLSVVDTYQLLTGKKELDDEEYYRLALLGWLIELLQA
FWLVSDDIMDESKTRRGQPCWYLKPKVGMIAIWDAFMLESGIYILLKKHFRQEKYYIDL
VELFHDISFKTELGQLVDLLTAPEDEVDLNRFSLDKHSFIVRYKTAYYSFYLPVVLAMYV
AGITNPKDLQQAMDVLIPLGEYFQVQDDYLDNFGDPEFIGKIGTDIQDNKCSWLVNKAL
QKATPEQRQILEDNYGVKDKSKELVIKKLYDDMKIEQDYLDYEEEVVGDIKKKIEQVDE
SRGFKKEVLNAFLAKIYKRQKGGAEAAAKEAAAKAGGSGGGSGGGGSGGSGGGGSGG
GGSMSDNSIATKILNFGHTCWKLQRPYAVKGMISIACGLFGKELLHNTNLISWGLMWKA
FFALVPILSFNFFAAIMNQIYDVDIDRINKPDLPLVSGEMSIETAWILSIIVALTGLIVTIKLK
SAPLFVFIYIFGIFAGFAYSVPPIRWKQNPSTNFLITISSHVGLAFTSYYASRAALGLPFELR
PSFTFLLAFMTVMGMTIAFAKDISDIEGDAKYGVSTVATKLGARNMTFVVSGVLLLNYV
AAILAGIIWPQAFNSNVMLLSHAILAFCLIFQTRELALANYASAPSRQFFEFIWLLYYAEY
FVYVFI
PKC4.33-F11-GPS1.1-F11-MPT21.3 (SEQ ID NO: 103)
MGEANKGVVKHVIILKFKEGITEAQKEEMFKTYVNLVNLVPAMKAVQW
GKLEVNNKLGNGGYTHIVESTFESVETIQDYIIHPAHVGFGDVYRSFWEKLLIFDYTPTIV
LPNSSYGGAEAAAKEAAAKAGGSGGGSGGGGSGGSGGGGSGGGGSMSKAKFESVFPRI
SEELVQLLRDEGLPQDAVQWFSDSLQYNCVGGKLNRGLSVVDTYQLLTGKKELDDEEY
YRLALLGWLIELLQAFWLVSDDIMDESKTRRGQPCWYLKPKVGMIAIWDAFMLESGIYI
LLKKHFRQEKYYIDLVELFHDISFKTELGQLVDLLTAPEDEVDLNRFSLDKHSFIVRYKTA
YYSFYLPVVLAMYVAGITNPKDLQQAMDVLIPLGEYFQVQDDYLDNFGDPEFIGKIGTDI
QDNKCSWLVNKALQKATPEQRQILEDNYGVKDKSKELVIKKLYDDMKIEQDYLDYEEE
VVGDIKKKIEQVDESRGFKKEVLNAFLAKIYKRQKGGAEAAAKEAAAKAGGSGGGSGG
GGSGGSGGGGSGGGGSMSDNSIATKILNFGHTCWKLQRPYAVKGMISIACGLFGKELLH
NTNLISWGLMWKAFFALVPILSFNFFAAIMNQIYDVDIDRINKPDLPLVSGEMSIETAWIL
SIIVALTGLIVTIKLKSAPLFVFIYIFGIFAGFAYSVPPIRWKQNPSTNFLITISSHVGLAFTSY
YASRAALGLPFELRPSFTFLLAFMTVMGMTIAFAKDISDIEGDAKYGVSTVATKLGARN
MTFVVSGVLLLNYVAAILAGIIWPQAFNSNVMLLSHAILAFCLIFQTRELALANYASAPSR
QFFEFIWLLYYAEYFVYVFI
PKC4.33-GPS1.1-F11-MPT21.3 (SEQ ID NO: 104)
MGEANKGVVKHVIILKFKEGITEAQKEEMFKTYVNLVNLVPAMKAVQW
GKLEVNNKLGNGGYTHIVESTFESVETIQDYIIHPAHVGFGDVYRSFWEKLLIFDYTPTIV
LPNSSYMSKAKFESVFPRISEELVQLLRDEGLPQDAVQWFSDSLQYNCVGGKLNRGLSV
VDTYQLLTGKKELDDEEYYRLALLGWLIELLQAFWLVSDDIMDESKTRRGQPCWYLKP
KVGMIAIWDAFMLESGIYILLKKHFRQEKYYIDLVELFHDISFKTELGQLVDLLTAPEDEV
DLNRFSLDKHSFIVRYKTAYYSFYLPVVLAMYVAGITNPKDLQQAMDVLIPLGEYFQVQ
DDYLDNFGDPEFIGKIGTDIQDNKCSWLVNKALQKATPEQRQILEDNYGVKDKSKELVI
KKLYDDMKIEQDYLDYEEEVVGDIKKKIEQVDESRGFKKEVLNAFLAKIYKRQKGGAE
AAAKEAAAKAGGSGGGSGGGGSGGSGGGGSGGGGSMSDNSIATKILNFGHTCWKLQRP
YAVKGMISIACGLFGKELLHNTNLISWGLMWKAFFALVPILSFNFFAAIMNQIYDVDIDRI
NKPDLPLVSGEMSIETAWILSIIVALTGLIVTIKLKSAPLFVFIYIFGIFAGFAYSVPPIRWKQ
NPSTNFLITISSHVGLAFTSYYASRAALGLPFELRPSFTFLLAFMTVMGMTIAFAKDISDIE
GDAKYGVSTVATKLGARNMTFVVSGVLLLNYVAAILAGIIWPQAFNSNVMLLSHAILAF
CLIFQTRELALANYASAPSRQFFEFIWLLYYAEYFVYVFI
HCS2 (SEQ ID NO: 105)
MASEENDLVFPSKEFSGQALVSSPQQYMEMHKRSMDDPAAFWSDIASEF
YWKQKWGDQVFSENLDVRKGPISIEWFKGGITNICYNCLDKNVEAGLGDKTAIHWEGN
ELGVDASLTYSELLQRVCQLANYLKDNGVKKGDAVVIYLPMLMELPIAMLACARIGAV
HSVVFAGFSADSLAQRIVDCKPNVILTCNAVKRGPKTINLKAIVDAALDQSSKDGVSVGI
CLTYDNSLATTRENTKWQNGRDVWWQDVISQYPTSCEVEWVDAEDPLFLLYTSGSTGK
PKGVLHTTGGYMIYTATTFKYAFDYKSTDVYWCTADCGWIGGHSYVTYGPMLNGATV
VVFEGAPNYPDPGRCWDIVDKYKVSIFYTAPTLVRSLMRDDDKFVTRHSRKSLRVLGSA
GEPINPSAWRWFFNVVGDSRCPISDTWGQTETGGFMITPLPGAWPQKPGSATFPFFGVQP
VIVDEKGNEIEGECSGYLCVKGSWPGAFRTLFGDHERYETTYFKPFAGYYFSGDGCSRD
KDGYYWLTGRVDDVINVSGHRIGTAEVESALVLHPQCAEAAVVGIEHEVKGQGIYAFVT
LLEGVPYSEELRKSLVLMVRNQIGAFAAPDRIHWAPGLPKTRSGKIMRRILRKIASRQLEE
LGDTSTLADPSVVDQLIALADV
PKC1.0 (SEQ ID NO: 106)
MAVKHLIVLKFKDEITEAQKEEFFKTYVNLVNIIPAMKDVYWGKDVTQK
NKEEGYTHIVEVTFESVETIQDYIIHPAHVGFGDVYRSFWEKLLIFDYTPRK
PKC1.1 (SEQ ID NO: 107)
MAVKHLIVLKFKDEITEAQKEEFFKTFVNLVNIIPAMKDVYWGKDVTQK
NKEEGYTHIVEVTFESVETIQDYIIHPAHVGFGDVYRSFWEKLLIFDYTPRK
PKC4.33 (SEQ ID NO: 108)
MGEANKGVVKHVIILKFKEGITEAQKEEMFKTYVNLVNLVPAMKAVQW
GKLEVNNKLGNGGYTHIVESTFESVETIQDYIIHPAHVGFGDVYRSFWEKLLIFDYTPTIV
LPNSSY
PKC11 (SEQ ID NO: 109)
MAVKHLIVLKFKDEITEAQKEEFFKTYVNLVNIIPAMKDVYWGKLEVNNKLGNGGYTHI
VEVTFESVETIQDYIIHPAHVGFGDVYRSFWEKLLIFDYTPRK
GPS1.1-F11-PKC1.1-F18-APT73.77 (SEQ ID NO: 110)
MSKAKFESVFPRISEELVQLLRDEGLPQDAVQWFSDSLQYNCVGGKLNR
GLSVVDTYQLLTGKKELDDEEYYRLALLGWLIELLQAFWLVSDDIMDESKTRRGQPCW
YLKPKVGMIAIWDAFMLESGIYILLKKHFRQEKYYIDLVELFHDISFKTELGQLVDLLTAP
EDEVDLNRFSLDKHSFIVRYKTAYYSFYLPVVLAMYVAGITNPKDLQQAMDVLIPLGEY
FQVQDDYLDNFGDPEFIGKIGTDIQDNKCSWLVNKALQKATPEQRQILEDNYGVKDKSK
ELVIKKLYDDMKIEQDYLDYEEEVVGDIKKKIEQVDESRGFKKEVLNAFLAKIYKRQKG
GAEAAAKEAAAKAGGSGGGSGGGGSGGSGGGGSGGGGSMAVKHLIVLKFKDEITEAQ
KEEFFKTFVNLVNIIPAMKDVYWGKDVTQKNKEEGYTHIVEVTFESVETIQDYIIHPAHV
GFGDVYRSFWEKLLIFDYTPRKGGGGSLEDPAVWEAGKVVAKGVGTADITATTSNGLIA
SSEEADNAATSMDEVYAAVEQTSRLLDVPCSPDRFEPVWKAFGDQLPDSHLVFSMAAG
EAHRGELDFDFSLRPEGADPYTTALEHGFIEPTDHPVGSVLAEVGKRFAIASYGVEYGVV
GGFKKSYAFFPLDDFPPLAQFAEVPSVPPCLAGHVETLTRLGFDDKVSIIGVNYRKNTLN
VYLAASAVDTGDKLALLRAFGYPEPDARVRQFIERSFRLYPTFNWDSSAAERICFAVHTQ
QPGELPAPHDEPTEAFARQVPHVYEGGREFVSGVALAPSGASYYKLAALYQKARRCLH
GPS1.1-F11-PKC4.33-F18-APT73.77 (SEQ ID NO: 111)
MSKAKFESVFPRISEELVQLLRDEGLPQDAVQWFSDSLQYNCVGGKLNR
GLSVVDTYQLLTGKKELDDEEYYRLALLGWLIELLQAFWLVSDDIMDESKTRRGQPCW
YLKPKVGMIAIWDAFMLESGIYILLKKHFRQEKYYIDLVELFHDISFKTELGQLVDLLTAP
EDEVDLNRFSLDKHSFIVRYKTAYYSFYLPVVLAMYVAGITNPKDLQQAMDVLIPLGEY
FQVQDDYLDNFGDPEFIGKIGTDIQDNKCSWLVNKALQKATPEQRQILEDNYGVKDKSK
ELVIKKLYDDMKIEQDYLDYEEEVVGDIKKKIEQVDESRGFKKEVLNAFLAKIYKRQKG
GAEAAAKEAAAKAGGSGGGSGGGGSGGSGGGGSGGGGSMGEANKGVVKHVIILKFKE
GITEAQKEEMFKTYVNLVNLVPAMKAVQWGKLEVNNKLGNGGYTHIVESTFESVETIQ
DYIIHPAHVGFGDVYRSFWEKLLIFDYTPTIVLPNSSYGGGGSLEDPAVWEAGKVVAKG
VGTADITATTSNGLIASSEEADNAATSMDEVYAAVEQTSRLLDVPCSPDRFEPVWKAFG
DQLPDSHLVFSMAAGEAHRGELDFDFSLRPEGADPYTTALEHGFIEPTDHPVGSVLAEVG
KRFAIASYGVEYGVVGGFKKSYAFFPLDDFPPLAQFAEVPSVPPCLAGHVETLTRLGFDD
KVSIIGVNYRKNTLNVYLAASAVDTGDKLALLRAFGYPEPDARVRQFIERSFRLYPTFNW
DSSAAERICFAVHTQQPGELPAPHDEPTEAFARQVPHVYEGGREFVSGVALAPSGASYY
KLAALYQKARRCLH
GPS1.1-PKC4.33-APT73.77 (SEQ ID NO: 112)
MSKAKFESVFPRISEELVQLLRDEGLPQDAVQWFSDSLQYNCVGGKLNR
GLSVVDTYQLLTGKKELDDEEYYRLALLGWLIELLQAFWLVSDDIMDESKTRRGQPCW
YLKPKVGMIAIWDAFMLESGIYILLKKHFRQEKYYIDLVELFHDISFKTELGQLVDLLTAP
EDEVDLNRFSLDKHSFIVRYKTAYYSFYLPVVLAMYVAGITNPKDLQQAMDVLIPLGEY
FQVQDDYLDNFGDPEFIGKIGTDIQDNKCSWLVNKALQKATPEQRQILEDNYGVKDKSK
ELVIKKLYDDMKIEQDYLDYEEEVVGDIKKKIEQVDESRGFKKEVLNAFLAKIYKRQKM
GEANKGVVKHVIILKFKEGITEAQKEEMFKTYVNLVNLVPAMKAVQWGKLEVNNKLG
NGGYTHIVESTFESVETIQDYIIHPAHVGFGDVYRSFWEKLLIFDYTPTIVLPNSSYMDEV
YAAVEQTSRLLDVPCSPDRFEPVWKAFGDQLPDSHLVFSMAAGEAHRGELDFDFSLRPE
GADPYTTALEHGFIEPTDHPVGSVLAEVGKRFAIASYGVEYGVVGGFKKSYAFFPLDDFP
PLAQFAEVPSVPPCLAGHVETLTRLGFDDKVSIIGVNYRKNTLNVYLAASAVDTGDKLA
LLRAFGYPEPDARVRQFIERSFRLYPTFNWDSSAAERICFAVHTQQPGELPAPHDEPTEAF
ARQVPHVYEGGREFVSGVALAPSGASYYKLAALYQKARRCLH
GPS1.1-F11-PKC11-F11-MPT4 (SEQ ID NO: 113)
MSKAKFESVFPRISEELVQLLRDEGLPQDAVQWFSDSLQYNCVGGKLNR
GLSVVDTYQLLTGKKELDDEEYYRLALLGWLIELLQAFWLVSDDIMDESKTRRGQPCW
YLKPKVGMIAIWDAFMLESGIYILLKKHFRQEKYYIDLVELFHDISFKTELGQLVDLLTAP
EDEVDLNRFSLDKHSFIVRYKTAYYSFYLPVVLAMYVAGITNPKDLQQAMDVLIPLGEY
FQVQDDYLDNFGDPEFIGKIGTDIQDNKCSWLVNKALQKATPEQRQILEDNYGVKDKSK
ELVIKKLYDDMKIEQDYLDYEEEVVGDIKKKIEQVDESRGFKKEVLNAFLAKIYKRQKG
GAEAAAKEAAAKAGGSGGGSGGGGSGGSGGGGSGGGGSMAVKHLIVLKFKDEITEAQ
KEEFFKTYVNLVNIIPAMKDVYWGKLEVNNKLGNGGYTHIVEVTFESVETIQDYIIHPAH
VGFGDVYRSFWEKLLIFDYTPRKGGAEAAAKEAAAKAGGSGGGSGGGGSGGSGGGGS
GGGGSMSDNSIATKILNFGHTCWKLQRPYVVKGMISIACGLFGRELFNNRHLFSWGLM
WKAFFALVPILSFNFFAAIMNQIYDVDIDRINKPDLPLVSGEMSIETAWILSIIVALTGLIVT
IKLKSAPLFVFIYIFGIFAGFAYSVPPIRWKQYPFTNFLITISSHVGLAFTSYSATTSALGLPF
VWRPAFSFIIAFMTVMGMTIAFAKDISDIEGDAKYGVSTVATKLGARNMTFVVSGVLLL
NYLVSISIGIIWPQVFKSNIMILSHAILAFCLIFQTRELALANYASAPSRQFFEFIWLLYYAE
YFVYVFI
GPS1.1-F11-PKC11-F11-MPT4.1 (SEQ ID NO: 114)
MSKAKFESVFPRISEELVQLLRDEGLPQDAVQWFSDSLQYNCVGGKLNR
GLSVVDTYQLLTGKKELDDEEYYRLALLGWLIELLQAFWLVSDDIMDESKTRRGQPCW
YLKPKVGMIAIWDAFMLESGIYILLKKHFRQEKYYIDLVELFHDISFKTELGQLVDLLTAP
EDEVDLNRFSLDKHSFIVRYKTAYYSFYLPVVLAMYVAGITNPKDLQQAMDVLIPLGEY
FQVQDDYLDNFGDPEFIGKIGTDIQDNKCSWLVNKALQKATPEQRQILEDNYGVKDKSK
ELVIKKLYDDMKIEQDYLDYEEEVVGDIKKKIEQVDESRGFKKEVLNAFLAKIYKRQKG
GAEAAAKEAAAKAGGSGGGSGGGGSGGSGGGGSGGGGSMAVKHLIVLKFKDEITEAQ
KEEFFKTYVNLVNIIPAMKDVYWGKLEVNNKLGNGGYTHIVEVTFESVETIQDYIIHPAH
VGFGDVYRSFWEKLLIFDYTPRKGGAEAAAKEAAAKAGGSGGGSGGGGSGGSGGGGS
GGGGSMSDNSIATKILNFGHTCWKLQRPYAVKGMISIACGLFGRELFNNRHLFSWGLM
WKAFFALVPILSFNFFAAIMNQIYDVDIDRINKPDLPLVSGEMSIETAWILSIIVALTGLIVT
IKLKSAPLFVFIYIFGIFAGFAYSVPPIRWKQYPFTNFLITISSHVGLAFTSYSATTSALGLPF
VWRPAFSFIIAFMTVMGMTIAFAKDISDIEGDAKYGVSTVATKLGARNMTFVVSGVLLL
NYLVSISIGIIWPQVFKSNIMILSHAILAFCLIFQTRELALANYASAPSRQFFEFIWLLYYAE
YFVYVFI

1-63. (canceled)

64. A recombinant prenyltransferase, wherein the recombinant prenyltransferase is a recombinant membrane-bound prenyltransferase (rMPT) or a recombinant aromatic prenyltransferase (APT) comprising an amino acid sequence having at least one amino acid modification as compared to a naturally occurring membrane-bound or aromatic prenyltransferase, wherein the rMPT comprises an amino acid sequence with at least 90% identity to SEQ ID NO: 22 (MPT21), SEQ ID NO: 52 (MPT4.1), SEQ ID NO: 23 (MPT26), or SEQ ID NO: 24 (MPT31), and the APT comprises an amino acid sequence with at least 90% identity to APT73.74 (SEQ ID NO: 37), APT73.77 (SEQ ID NO: 38), or APT89.38 (SEQ ID NO: 39), or a functional fragment or variant thereof.

65. The recombinant prenyltransferase of claim 64, wherein the rMPT comprises an amino acid sequence having at least one amino acid modification as compared to SEQ ID NO: 22 (MPT21), wherein the at least one amino acid modification comprises a deletion, insertion or substitution at an amino acid position selected from R22, P23, Y24, V25, V26, K27, G28, M29, S31, A33, F66, N67, A70, A71, D80, I81, I84, N85, K86, P87, D88, L89, L91. V92, Y139, S140, F154, L155, I158, S159, S160, V162, T194, V195, G197, M198, I200, A201, F202, A203, K204, D208, I209, G211, D212, L267, A268, L271, T275, L278, N282. A284, S285, R289, F292, I295, W296, L298, and Y299.

66. The recombinant prenyltransferase of claim 64, wherein the rMPT comprises an amino acid sequence having at least one amino acid modification as compared to SEQ ID 52 (MPT4.1), wherein the at least one amino acid modification comprises a deletion, insertion or substitution at an amino acid position selected from R22, P23, Y24, V25, V26, K27, G28, M29, S31, A33, F66, N67, A70, A71, D80, I81, I84, N85, K86, P87, D88, L89, L91, V92, Y139, S140, F154, L155, I158, S159, S160, V162, T194, V195, G197, M198, I200, A201, F202, A203, K204, D208, I209, G211, D212, L267, A268, L271, T275, L278, N282, A284, S285, R289, F292, I295, W296, L298, and Y299.

67. The recombinant prenyltransferase of claim 64, wherein the rMPT comprises an amino acid sequence having at least one amino acid modification as compared to SEQ ID NO: 23 (MPT26), wherein the at least one amino acid modification is selected from R22, P23, Y24, V25, V26, K27, G28, M29, S31, A33, F66, N67, A70, A71, I81, I84, N85, K86, P87, D88, L89, L91, Y139, S140, F154, L155, I158, S159, S160, V162, T194, V195, G197, M198, I200, A201, F202, A203, K204, D208, I209, G211, D212, L267, A268, L271, T275, FL278, N282, A284, D284, S285P285, R289, F292, I295, W296, L298, and Y299.

68. The recombinant prenyltransferase of claim 64, wherein the rMPT comprises an amino acid sequence having at least one amino acid modification as compared to SEQ ID NO: 24 (MPT31), wherein the at least one amino acid modification is selected from R22, P23, Y24, V25, V26, K27, G28, M29, S31, A33, F66, N67, A70, A71, D80 (H80-MPT31), I81, I84, N85, K86, P87, D88, L89, L91, A92, Y139, S140, F154, L155, I158, S159, S160, V162, T194, V195, G197, M198, I200, A201, F202, A203, K204, D208, I209, G211, D212, L267, A268, L271, T275, FL278, N282, A284D284, S285P285, R289, F292, I295, W296, L298, and Y299.

69. A cell comprising the recombinant prenyltransferase of claim 64, wherein the cell is capable of producing CBGA in the presence of GPP and OA, or is capable of producing CBGVA in the presence of GPP and DVA.

70. The cell of claim 69, wherein the cell expresses an exogenous membrane transporter that improves OA or DVA uptake.

71. The cell of claim 70, wherein one or more genes in the cell encoding a protein that exports OA or DVA is down-regulated or deactivated.

72. A cell of claim 69, wherein the cell is capable of forming acetyl-CoA from a carboxylic acid.

73. The cell of claim 69, wherein the cell encodes an exogenous hexanoyl-CoA synthetase and/or butyryl-CoA synthase.

74. The cell of claim 69, wherein the cell is a yeast cell.

75. The recombinant prenyltransferase of claim 64, wherein the rMPT or APT is fused with a polypeptide having Geranyl diphosphate synthase activity.

76. The recombinant prenyltransferase of claim 75, wherein the polypeptide having Geranyl diphosphate synthase activity comprises a polypeptide of SEQ ID NO: 4 (GPS1.1), SEQ ID NO: 5 (GPS2), or SEQ ID NO: 6 (GPS3), or a functional fragment or functional variant thereof.

77. The recombinant prenyltransferase of claim 76, wherein the fusion protein further comprises a linker polypeptide between the polypeptide having Geranyl diphosphate synthase activity and the polypeptide having prenyltransferase activity.

78. The fusion protein of claim 75, wherein the fusion protein comprises the polypeptide sequence of GPS1.1-F11-MPT4 (SEQ ID NO: 16), GPS1.1-F5-MPT4 (SEQ ID NO: 17), GPS1.1-F9-MPT4 (SEQ ID NO: 18), GPS1.1-F10-MPT4 (SEQ ID NO: 19), GPS3-F11-MPT4 (SEQ ID NO: 20), GPS2-F11-MPT4 (SEQ ID NO: 21), GPS1.1-F5-MPT4.1 (SEQ ID NO: 54), GPS1.1-F9-MPT4.1 (SEQ ID NO: 53),GPS1.1-F10-MPT4.1 (SEQ ID NO: 55), GPS1.1-F11-MPT4.1 (SEQ ID NO: 56), GPS3-F11-MPT4.1 (SEQ ID NO: 57), GPS2-F11-MPT4.1 (SEQ ID NO: 58), GPS1.1-F16-APT73.74 (SEQ ID NO: 59), APT73.74-F17-GPS1.1 (SEQ ID NO: 60), GPS1.1-F18-APT73.74 (SEQ ID NO: 61), APT73.74-F18-GPS1.1 (SEQ ID NO: 62), GPS1.1-F16-APT73.77 (SEQ ID NO: 63), APT73.77-F17-GPS1.1 (SEQ ID NO: 64), GPS1.1-F18-APT73.77 (SEQ ID NO: 65), APT73.77-F18-GPS1.1 (SEQ ID NO: 66), GPS1.1-F16-APT89.38 (SEQ ID NO: 67), APT89.38-F17-GPS1.1 (SEQ ID NO: 68), GPS1.1-F18-APT89.38 (SEQ ID NO: 69), APT89.38-F18-GPS1.1 (SEQ ID NO: 70), or a functional fragment or variant thereof.

79. A fusion protein of claim 75, further comprising a polypeptide having polyketide cyclase (PKC) activity.

80. The fusion protein of claim 79, wherein the polypeptide having polyketide cyclase activity comprises a polypeptide sequence having at least 90% identity to the polypeptide sequence of PKC1.0 (SEQ ID NO: 106), PKC1.1 (SEQ ID NO: 107), or PKC4.33 (SEQ ID NO 108), SEQ ID NO: 107 (PKC1.1) or SEQ ID NO: 108 (PKC4.33) or SEQ ID 109 (PKC11).

81. The fusion protein of claim 79, wherein the fusion protein comprising all three of the polypeptides having polyketide cyclase activity, prenyltransferase activity and geranyl diphosphate synthase activity, exhibits improved production of CBGA and/or CBGVA as compared to a control membrane bound prenyltransferase or as compared to a fusion protein comprising polypeptides having prenyltransferase and geranyl diphosphate synthase activities and which lacks a polypeptide having polyketide cyclase activity.

82. The fusion protein of claim 81, wherein the fusion protein comprises the polypeptide sequence of GPS1.1-PKC4.33-APT73.77 (SEQ ID NO: 112), GPS1.1-F11-PKC4.33-F18-APT73.77 (SEQ ID NO: 111), GPS1.1-F11-PKC1.1-F18-APT73.77 (SEQ ID NO: 110), PKC4.33-GPS1.1-F11-MPT21.3 (SEQ ID NO: 104), PKC4.33-F11-GPS1.1-F11-MPT21.3 (SEQ ID NO: 103), PKC1.1-F11-GPS1.1-F11-MPT21.3 (SEQ ID NO: 102), PKC4.33-GPS1.1-F11-MPT4.1 (SEQ ID NO: 101), PKC4.33-F11-GPS1.1-F11-MPT4.1 (SEQ ID NO: 100), PKC1.1-F11-GPS1.1-F11-MPT4.1 (SEQ ID NO: 99), GPS1.1-PKC4.33-MPT21.3 (SEQ ID NO: 98), GPS1.1-PKC4.33-MPT4.1 (SEQ ID NO: 97), GPS1.1-F11-PKC4.33-F11-MPT21.3 (SEQ ID NO: 96), GPS1.1-F11-PKC4.33-F11-MPT4.1 (SEQ ID NO: 95), GPS1.1-F11-PKC1.1-F11-MPT21.3 (SEQ ID NO: 94), GPS1.1-F11-PKC1.1-F11-MPT4.1 (SEQ ID NO: 93), or GPS1.1-F11-PKC1.1-F11-MPT4 (SEQ ID NO: 92), GPS1.1-F11-PKC11-F11-MPT4 (SEQ ID NO: 113) or GPS1.1-F11-PKC11-F11-MPT4.1 (SEQ ID NO: 114).

83. A method of producing CBGA or CBGVA comprising contacting the cell of claim 64 with a carbon source under suitable conditions to produce CBGA or CBGVA.


SEQUENCE LISTING (114 sequences)

SEQ ID NO: 1 - protein, 395 aa, Unknown
  MPT1 (membrane-bound prenyltransferase)
MGLSSVCTFSFQTNYHTLLNPHNNNPKTSLLCYRHPKTPIKYSYNNFPSKHCSTKSFHLQNKCSESLSIAKNSIRAATTNQTEPPESDNHSVATKILNFGKACWKLQRPYTIIAFTSCACGLFGKELLHNTNLISWSLMFKAFFFLVAVLCIASFTTTINQIYDLHIDRINKPDLPLASGEISVNTAWIMSIIVALFGLIITIKMKGGPLYIFGYCFGIFGGIVYSVPPFRWKQNPSTAFLLNFLAHIITNFTFYYASRAALGLPFELRPSFTFLLAFMKSMGSALALIKDASDVEGDTKFGISTLASKYGSRNLTLFCSGIVLLSYVAAILAGIIWPQAFNSNVMLLSHAILAFWLILQTRDFALTNYDPEAGRRFYEFMWKLYYAEYLVYVFI

SEQ ID NO: 2 - protein, 309 aa, Unknown
  MPT4 (membrane-bound prenyltransferase)
MSDNSIATKILNFGHTCWKLQRPYVVKGMISIACGLFGRELFNNRHLFSWGLMWKAFFALVPILSFNFFAAIMNQIYDVDIDRINKPDLPLVSGEMSIETAWILSIIVALTGLIVTIKLKSAPLFVFIYIFGIFAGFAYSVPPIRWKQYPFTNFLITISSHVGLAFTSYSATTSALGLPFVWRPAFSFIIAFMTVMGMTIAFAKDISDIEGDAKYGVSTVATKLGARNMTFVVSGVLLLNYLVSISIGIIWPQVFKSNIMILSHAILAFCLIFQTRELALANYASAPSRQFFEFIWLLYYAEYFVYVFI

SEQ ID NO: 3 - skipped

SEQ ID NO: 4 - protein, 344 aa, Unknown
  GPS1.1 (geranyl diphosphate synthase)
MSKAKFESVFPRISEELVQLLRDEGLPQDAVQWFSDSLQYNCVGGKLNRGLSVVDTYQLLTGKKELDDEEYYRLALLGWLIELLQAFWLVSDDIMDESKTRRGQPCWYLKPKVGMIAIWDAFMLESGIYILLKKHFRQEKYYIDLVELFHDISFKTELGQLVDLLTAPEDEVDLNRFSLDKHSFIVRYKTAYYSFYLPVVLAMYVAGITNPKDLQQAMDVLIPLGEYFQVQDDYLDNFGDPEFIGKIGTDIQDNKCSWLVNKALQKATPEQRQILEDNYGVKDKSKELVIKKLYDDMKIEQDYLDYEEEVVGDIKKKIEQVDESRGFKKEVLNAFLAKIYKRQK

SEQ ID NO: 5 - protein, 371 aa, Unknown
  GPS2 (geranyl diphosphate synthase
MKDVSLSSFDAHDLDLDKFPEVVRDRLTQFLDAQELTIADIGAPVTDAVAHLRSFVLNGGKRIRPLYAWAGFLAAQGHKNSSEKLESVLDAAASLEFIQACALIHDDIIDSSDTRRGAPTVHRAVEADHRANNFEGDPEHFGVSVSILAGDMALVWAEDMLQDSGLSAEALARTRDAWRGMRTEVIGGQLLDIYLESHANESVELADSVNRFKTAAYTIARPLHLGASIAGGSPQLIDALLHYGHDIGIAFQLRDDLLGVFGDPAITGKPAGDDIREGKRTVLLALALQRADKQSPEAATAIRAGVGKVTSPEDIAVITEHIRATGAEEEVEQRISQLTESGLAHLDDVDIPDEVRAQLRALAIRSTERRM

SEQ ID NO: 6 - protein, 310 aa, Unknown
  GPS3 (geranyl diphosphate synthase)
MQLLNPPQKGKKAVEFDFNKYMDSKAMTVNEALNKAIPLRYPQKIYESMRYSLLAGGKRVRPVLCIAACELVGGTEELAIPTACAIEMIHTMSLMHDDLPCIDNDDLRRGKPTNHKIFGEDTAVTAGNALHSYAFEHIAVSTSKTVGADRILRMVSELGRATGSEGVMGGQMVDIASEGDPSIDLQTLEWIHIHKTAMLLECSVVCGAIIGGASEIVIERARRYARCVGLLFQVVDDILDVTKSSDELGKTAGKDLISDKATYPKLMGLEKAKEFSDELLNRAKGELSCFDPVKAAPLLGLADYVAFRQN

SEQ ID NO: 7 - protein, 33 aa, Artificial
  F1 linker
GGGGSGGGGSAEAAAKAEAAAKAGGGGSGGGGS

SEQ ID NO: 8 - protein, 29 aa, Artificial
  F2 linker
GGAEAAAKEAAAKAGGSGGGSGGGGSGGS

SEQ ID NO: 9 - protein, 39 aa, Artificial
  F3 linker
GGAEAAAKEAAAKAAEAAAKEAAAKAGGGSPGPGPGGGS

SEQ ID NO: 10 - protein, 36 aa, Artificial
  F4 linker
GSSSSSSGSSSSSSGSSSSSSGSSSSSSGSSSSSSG

SEQ ID NO: 11 - protein, 15 aa, Artificial
  F5 linker
GGGGSGGGGSGGGGS

SEQ ID NO: 12 - protein, 19 aa, Artificial
  F6 linker
GGEAAAKEAAAKEAAAKGG

SEQ ID NO: 13 - protein, 21 aa, Artificial
  F7 linker
GGAEAAAKEAAAKAPAPAPAG

SEQ ID NO: 14 - protein, 11 aa, Artificial
  F8 linker
GTPTPTPTPTG

SEQ ID NO: 15 - protein, 30 aa, Artificial
  F9 linker
GGGGSGGGGSGGGGSGGGGSGGGGSGGGGS

SEQ ID NO: 16 - protein, 692 aa, Artificial
  GPS1.1-F11-MPT4, geranyl diphosphate synthase, membrane-bound prenyltransferase fusion protein
MSKAKFESVFPRISEELVQLLRDEGLPQDAVQWFSDSLQYNCVGGKLNRGLSVVDTYQLLTGKKELDDEEYYRLALLGWLIELLQAFWLVSDDIMDESKTRRGQPCWYLKPKVGMIAIWDAFMLESGIYILLKKHFRQEKYYIDLVELFHDISFKTELGQLVDLLTAPEDEVDLNRFSLDKHSFIVRYKTAYYSFYLPVVLAMYVAGITNPKDLQQAMDVLIPLGEYFQVQDDYLDNFGDPEFIGKIGTDIQDNKCSWLVNKALQKATPEQRQILEDNYGVKDKSKELVIKKLYDDMKIEQDYLDYEEEVVGDIKKKIEQVDESRGFKKEVLNAFLAKIYKRQKGGAEAAAKEAAAKAGGSGGGSGGGGSGGSGGGGSGGGGSMSDNSIATKILNFGHTCWKLQRPYVVKGMISIACGLFGRELFNNRHLFSWGLMWKAFFALVPILSFNFFAAIMNQIYDVDIDRINKPDLPLVSGEMSIETAWILSIIVALTGLIVTIKLKSAPLFVFIYIFGIFAGFAYSVPPIRWKQYPFTNFLITISSHVGLAFTSYSATTSALGLPFVWRPAFSFIIAFMTVMGMTIAFAKDISDIEGDAKYGVSTVATKLGARNMTFVVSGVLLLNYLVSISIGIIWPQVFKSNIMILSHAILAFCLIFQTRELALANYASAPSRQFFEFIWLLYYAEYFVYVFI

SEQ ID NO: 17 - protein, 668 aa, Artificial
  GPS1.1-F5-MPT4, geranyl diphosphate synthase, membrane-bound prenyltransferase fusion protein
MSKAKFESVFPRISEELVQLLRDEGLPQDAVQWFSDSLQYNCVGGKLNRGLSVVDTYQLLTGKKELDDEEYYRLALLGWLIELLQAFWLVSDDIMDESKTRRGQPCWYLKPKVGMIAIWDAFMLESGIYILLKKHFRQEKYYIDLVELFHDISFKTELGQLVDLLTAPEDEVDLNRFSLDKHSFIVRYKTAYYSFYLPVVLAMYVAGITNPKDLQQAMDVLIPLGEYFQVQDDYLDNFGDPEFIGKIGTDIQDNKCSWLVNKALQKATPEQRQILEDNYGVKDKSKELVIKKLYDDMKIEQDYLDYEEEVVGDIKKKIEQVDESRGFKKEVLNAFLAKIYKRQKGGGGSGGGGSGGGGSMSDNSIATKILNFGHTCWKLQRPYVVKGMISIACGLFGRELFNNRHLFSWGLMWKAFFALVPILSFNFFAAIMNQIYDVDIDRINKPDLPLVSGEMSIETAWILSIIVALTGLIVTIKLKSAPLFVFIYIFGIFAGFAYSVPPIRWKQYPFTNFLITISSHVGLAFTSYSATTSALGLPFVWRPAFSFIIAFMTVMGMTIAFAKDISDIEGDAKYGVSTVATKLGARNMTFVVSGVLLLNYLVSISIGIIWPQVFKSNIMILSHAILAFCLIFQTRELALANYASAPSRQFFEFIWLLYYAEYFVYVFI

SEQ ID NO: 18 - protein, 683 aa, Artificial
  GPS1.1-F9-MPT4, geranyl diphosphate synthase, membrane-bound prenyltransferase fusion protein
MSKAKFESVFPRISEELVQLLRDEGLPQDAVQWFSDSLQYNCVGGKLNRGLSVVDTYQLLTGKKELDDEEYYRLALLGWLIELLQAFWLVSDDIMDESKTRRGQPCWYLKPKVGMIAIWDAFMLESGIYILLKKHFRQEKYYIDLVELFHDISFKTELGQLVDLLTAPEDEVDLNRFSLDKHSFIVRYKTAYYSFYLPVVLAMYVAGITNPKDLQQAMDVLIPLGEYFQVQDDYLDNFGDPEFIGKIGTDIQDNKCSWLVNKALQKATPEQRQILEDNYGVKDKSKELVIKKLYDDMKIEQDYLDYEEEVVGDIKKKIEQVDESRGFKKEVLNAFLAKIYKRQKGGGGSGGGGSGGGGSGGGGSGGGGSGGGGSMSDNSIATKILNFGHTCWKLQRPYVVKGMISIACGLFGRELFNNRHLFSWGLMWKAFFALVPILSFNFFAAIMNQIYDVDIDRINKPDLPLVSGEMSIETAWILSIIVALTGLIVTIKLKSAPLFVFIYIFGIFAGFAYSVPPIRWKQYPFTNFLITISSHVGLAFTSYSATTSALGLPFVWRPAFSFIIAFMTVMGMTIAFAKDISDIEGDAKYGVSTVATKLGARNMTFVVSGVLLLNYLVSISIGIIWPQVFKSNIMILSHAILAFCLIFQTRELALANYASAPSRQFFEFIWLLYYAEYFVYVFI

SEQ ID NO: 19 - protein, 705 aa, Artificial
  GPS1.1-F10-MPT4, geranyl diphosphate synthase, membrane-bound prenyltransferase fusion protein
MSKAKFESVFPRISEELVQLLRDEGLPQDAVQWFSDSLQYNCVGGKLNRGLSVVDTYQLLTGKKELDDEEYYRLALLGWLIELLQAFWLVSDDIMDESKTRRGQPCWYLKPKVGMIAIWDAFMLESGIYILLKKHFRQEKYYIDLVELFHDISFKTELGQLVDLLTAPEDEVDLNRFSLDKHSFIVRYKTAYYSFYLPVVLAMYVAGITNPKDLQQAMDVLIPLGEYFQVQDDYLDNFGDPEFIGKIGTDIQDNKCSWLVNKALQKATPEQRQILEDNYGVKDKSKELVIKKLYDDMKIEQDYLDYEEEVVGDIKKKIEQVDESRGFKKEVLNAFLAKIYKRQKGGAEAAAKEAAAKAAEAAAKEAAAKAAEAAAKEAAAKAAEAAAKEAAAKAGGMSDNSIATKILNFGHTCWKLQRPYVVKGMISIACGLFGRELFNNRHLFSWGLMWKAFFALVPILSFNFFAAIMNQIYDVDIDRINKPDLPLVSGEMSIETAWILSIIVALTGLIVTIKLKSAPLFVFIYIFGIFAGFAYSVPPIRWKQYPFTNFLITISSHVGLAFTSYSATTSALGLPFVWRPAFSFIIAFMTVMGMTIAFAKDISDIEGDAKYGVSTVATKLGARNMTFVVSGVLLLNYLVSISIGIIWPQVFKSNIMILSHAILAFCLIFQTRELALANYASAPSRQFFEFIWLLYYAEYFVYVFI

SEQ ID NO: 20 - protein, 658 aa, Artificial
  GPS3-F11-MPT4, geranyl diphosphate synthase, membrane-bound prenyltransferase fusion protein
MQLLNPPQKGKKAVEFDFNKYMDSKAMTVNEALNKAIPLRYPQKIYESMRYSLLAGGKRVRPVLCIAACELVGGTEELAIPTACAIEMIHTMSLMHDDLPCIDNDDLRRGKPTNHKIFGEDTAVTAGNALHSYAFEHIAVSTSKTVGADRILRMVSELGRATGSEGVMGGQMVDIASEGDPSIDLQTLEWIHIHKTAMLLECSVVCGAIIGGASEIVIERARRYARCVGLLFQVVDDILDVTKSSDELGKTAGKDLISDKATYPKLMGLEKAKEFSDELLNRAKGELSCFDPVKAAPLLGLADYVAFRQNGGAEAAAKEAAAKAGGSGGGSGGGGSGGSGGGGSGGGGSMSDNSIATKILNFGHTCWKLQRPYVVKGMISIACGLFGRELFNNRHLFSWGLMWKAFFALVPILSFNFFAAIMNQIYDVDIDRINKPDLPLVSGEMSIETAWILSIIVALTGLIVTIKLKSAPLFVFIYIFGIFAGFAYSVPPIRWKQYPFTNFLITISSHVGLAFTSYSATTSALGLPFVWRPAFSFIIAFMTVMGMTIAFAKDISDIEGDAKYGVSTVATKLGARNMTFVVSGVLLLNYLVSISIGIIWPQVFKSNIMILSHAILAFCLIFQTRELALANYASAPSRQFFEFIWLLYYAEYFVYVFI

SEQ ID NO: 21 - protein, 719 aa, Artificial
  GPS2-F11-MPT4, geranyl diphosphate synthase, membrane-bound prenyltransferase fusion protein
MKDVSLSSFDAHDLDLDKFPEVVRDRLTQFLDAQELTIADIGAPVTDAVAHLRSFVLNGGKRIRPLYAWAGFLAAQGHKNSSEKLESVLDAAASLEFIQACALIHDDIIDSSDTRRGAPTVHRAVEADHRANNFEGDPEHFGVSVSILAGDMALVWAEDMLQDSGLSAEALARTRDAWRGMRTEVIGGQLLDIYLESHANESVELADSVNRFKTAAYTIARPLHLGASIAGGSPQLIDALLHYGHDIGIAFQLRDDLLGVFGDPAITGKPAGDDIREGKRTVLLALALQRADKQSPEAATAIRAGVGKVTSPEDIAVITEHIRATGAEEEVEQRISQLTESGLAHLDDVDIPDEVRAQLRALAIRSTERRMGGAEAAAKEAAAKAGGSGGGSGGGGSGGSGGGGSGGGGSMSDNSIATKILNFGHTCWKLQRPYVVKGMISIACGLFGRELFNNRHLFSWGLMWKAFFALVPILSFNFFAAIMNQIYDVDIDRINKPDLPLVSGEMSIETAWILSIIVALTGLIVTIKLKSAPLFVFIYIFGIFAGFAYSVPPIRWKQYPFTNFLITISSHVGLAFTSYSATTSALGLPFVWRPAFSFIIAFMTVMGMTIAFAKDISDIEGDAKYGVSTVATKLGARNMTFVVSGVLLLNYLVSISIGIIWPQVFKSNIMILSHAILAFCLIFQTRELALANYASAPSRQFFEFIWLLYYAEYFVYVFI

SEQ ID NO: 22 - protein, 309 aa, Artificial
  MPT21 (recombinant membrane-bound prenyltransferase)
MSDNSIATKILNFGHTCWKLQRPYVVKGMISIACGLFGKELLHNTNLISWGLMWKAFFALVPILSFNFFAAIMNQIYDVDIDRINKPDLPLVSGEMSIETAWILSIIVALTGLIVTIKLKSAPLFVFIYIFGIFAGFAYSVPPIRWKQNPSTNFLITISSHVGLAFTSYYASRAALGLPFELRPSFTFLLAFMTVMGMTIAFAKDISDIEGDAKYGVSTVATKLGARNMTFVVSGVLLLNYVAAILAGIIWPQAFNSNVMLLSHAILAFCLIFQTRELALANYASAPSRQFFEFIWLLYYAEYFVYVFI

SEQ ID NO: 23 - protein, 309 aa, Artificial
  MPT26 (recombinant membrane-bound prenyltransferase)
MSDNSIATKILNFGHTCWKLQRPYVVKGMISIACGLFGKELLHNTNLISWGLMWKAFFALVPILSFNFFAAIMNQIYDVDIDRINKPDLPLVSGEMSIETAWILSIIVALTGLIVTIKLKSAPLFVFIYIFGIFAGFAYSVPPIRWKQNPSTNFLITISSHVGLAFTSYSATTSALGLPFVWRPAFSFIIAFMTVMGMTIAFAKDISDIEGDAKYGVSTVATKLGARNMTFVVSGVLLLNYVAAILAGIIWPQAFNSNVMLLSHAILAFWLILQTRDFALTNYDPEAGRRFFEFIWLLYYAEYFVYVFI

SEQ ID NO: 24 - protein, 309 aa, Artificial
  MPT31 (recombinant membrane-bound prenyltransferase)
MSDNSIATKILNFGHACWKLQRPYVVKGMISIACGLFGRELLHNTNLISWGLMWKAFFALVPILSFNFFAAIMNQIYDLHIDRINKPDLPLASGEISVNTAWIMSIIVALTGLIVTIKLKSAPLFVFIYIFGIFAGFAYSVPPIRWKQNPSTNFLITISSHVGLAFTSYYASRAALGLPFELRPSFTFLLAFMTVMGMTIAFAKDISDIEGDAKYGVSTVATKLGARNMTFVVSGVLLLNYLVSISIGIIWPQVFKSNIMILSHAILAFWLILQTRDFALTNYDPEAGRRFFEFIWLLYYAEYLVYVFI

SEQ ID NO: 25 - protein, 692 aa, Artificial
  GPS1.1-F11-MPT21, geranyl diphosphate synthase, recombinant membrane-bound prenyltransferase fusion protein
MSKAKFESVFPRISEELVQLLRDEGLPQDAVQWFSDSLQYNCVGGKLNRGLSVVDTYQLLTGKKELDDEEYYRLALLGWLIELLQAFWLVSDDIMDESKTRRGQPCWYLKPKVGMIAIWDAFMLESGIYILLKKHFRQEKYYIDLVELFHDISFKTELGQLVDLLTAPEDEVDLNRFSLDKHSFIVRYKTAYYSFYLPVVLAMYVAGITNPKDLQQAMDVLIPLGEYFQVQDDYLDNFGDPEFIGKIGTDIQDNKCSWLVNKALQKATPEQRQILEDNYGVKDKSKELVIKKLYDDMKIEQDYLDYEEEVVGDIKKKIEQVDESRGFKKEVLNAFLAKIYKRQKGGAEAAAKEAAAKAGGSGGGSGGGGSGGSGGGGSGGGGSMSDNSIATKILNFGHTCWKLQRPYVVKGMISIACGLFGKELLHNTNLISWGLMWKAFFALVPILSFNFFAAIMNQIYDVDIDRINKPDLPLVSGEMSIETAWILSIIVALTGLIVTIKLKSAPLFVFIYIFGIFAGFAYSVPPIRWKQNPSTNFLITISSHVGLAFTSYYASRAALGLPFELRPSFTFLLAFMTVMGMTIAFAKDISDIEGDAKYGVSTVATKLGARNMTFVVSGVLLLNYVAAILAGIIWPQAFNSNVMLLSHAILAFCLIFQTRELALANYASAPSRQFFEFIWLLYYAEYFVYVFI

SEQ ID NO: 26 - protein, 692 aa, Artificial
  GPS1.1-F11-MPT26, geranyl diphosphate synthase, recombinant membrane-bound prenyltransferase fusion protein
MSKAKFESVFPRISEELVQLLRDEGLPQDAVQWFSDSLQYNCVGGKLNRGLSVVDTYQLLTGKKELDDEEYYRLALLGWLIELLQAFWLVSDDIMDESKTRRGQPCWYLKPKVGMIAIWDAFMLESGIYILLKKHFRQEKYYIDLVELFHDISFKTELGQLVDLLTAPEDEVDLNRFSLDKHSFIVRYKTAYYSFYLPVVLAMYVAGITNPKDLQQAMDVLIPLGEYFQVQDDYLDNFGDPEFIGKIGTDIQDNKCSWLVNKALQKATPEQRQILEDNYGVKDKSKELVIKKLYDDMKIEQDYLDYEEEVVGDIKKKIEQVDESRGFKKEVLNAFLAKIYKRQKGGAEAAAKEAAAKAGGSGGGSGGGGSGGSGGGGSGGGGSMSDNSIATKILNFGHTCWKLQRPYVVKGMISIACGLFGKELLHNTNLISWGLMWKAFFALVPILSFNFFAAIMNQIYDVDIDRINKPDLPLVSGEMSIETAWILSIIVALTGLIVTIKLKSAPLFVFIYIFGIFAGFAYSVPPIRWKQNPSTNFLITISSHVGLAFTSYSATTSALGLPFVWRPAFSFIIAFMTVMGMTIAFAKDISDIEGDAKYGVSTVATKLGARNMTFVVSGVLLLNYVAAILAGIIWPQAFNSNVMLLSHAILAFWLILQTRDFALTNYDPEAGRRFFEFIWLLYYAEYFVYVFI

SEQ ID NO: 27 - protein, 692 aa, Artificial
  GPS1.1-F11-MPT31, geranyl diphosphate synthase, recombinant membrane-bound prenyltransferase fusion protein
MSKAKFESVFPRISEELVQLLRDEGLPQDAVQWFSDSLQYNCVGGKLNRGLSVVDTYQLLTGKKELDDEEYYRLALLGWLIELLQAFWLVSDDIMDESKTRRGQPCWYLKPKVGMIAIWDAFMLESGIYILLKKHFRQEKYYIDLVELFHDISFKTELGQLVDLLTAPEDEVDLNRFSLDKHSFIVRYKTAYYSFYLPVVLAMYVAGITNPKDLQQAMDVLIPLGEYFQVQDDYLDNFGDPEFIGKIGTDIQDNKCSWLVNKALQKATPEQRQILEDNYGVKDKSKELVIKKLYDDMKIEQDYLDYEEEVVGDIKKKIEQVDESRGFKKEVLNAFLAKIYKRQKGGAEAAAKEAAAKAGGSGGGSGGGGSGGSGGGGSGGGGSMSDNSIATKILNFGHACWKLQRPYVVKGMISIACGLFGRELLHNTNLISWGLMWKAFFALVPILSFNFFAAIMNQIYDLHIDRINKPDLPLASGEISVNTAWIMSIIVALTGLIVTIKLKSAPLFVFIYIFGIFAGFAYSVPPIRWKQNPSTNFLITISSHVGLAFTSYYASRAALGLPFELRPSFTFLLAFMTVMGMTIAFAKDISDIEGDAKYGVSTVATKLGARNMTFVVSGVLLLNYLVSISIGIIWPQVFKSNIMILSHAILAFWLILQTRDFALTNYDPEAGRRFFEFIWLLYYAEYLVYVFI

SEQ ID NO: 28 - protein, 52 aa, Artificial
  F10 linker
GGAEAAAKEAAAKAAEAAAKEAAAKAAEAAAKEAAAKAAEAAAKEAAAKAGG

SEQ ID NO: 29 - protein, 39 aa, Artificial
  F11 linker
GGAEAAAKEAAAKAGGSGGGSGGGGSGGSGGGGSGGGGS

SEQ ID NO: 30 - protein, 10 aa, Artificial
  F12 linker
GGGGSGGGGS

SEQ ID NO: 31 - protein, 17 aa, Artificial
  F13 linker
GGSGSAGSAAGSGEFGG

SEQ ID NO: 32 - protein, 33 aa, Artificial
  F14 linker
GGAEAAAKEAAAKAPAPAPAEAAAKEAAAKAGG

SEQ ID NO: 33 - protein, 22 aa, Artificial
  F15 linker
GGSGGAEAAAKEAAAKAGGSGG

SEQ ID NO: 34 - protein, 17 aa, Artificial
  F16 linker
GGGSGGGSGGGSGGGGS

SEQ ID NO: 35 - protein, 5 aa, Artificial
  F17 linker
GGGGS

SEQ ID NO: 36 - protein, 48 aa, Artificial
  F18 linker
GGGGSLEDPAVWEAGKVVAKGVGTADITATTSNGLIASSEEADNAATS

SEQ ID NO: 37 - protein, 285 aa, Artificial
  APT73.74 (mutant soluble aromatic prenyltransferase)
MDEVYAAVEQTSRLLDVPCSPDRFEPVWKAFGDQLPDSHLVFSMAAGEAHRGELDFDFSLRPEGADPYTTALEHGFIEPTDHPVGSVLAEVGKRFAIASYGVEYGVVGGFKKSYAFFPLDDFPPLAQFAEVPSVPPCLAGHVETLTRLGFDDKVSIIGVNYRKNTLNVYLAASAVDTGDKLALLRAFGYPEPDARVRQFIERSFRLYPTFNWDSSAAERICFAVHTQQPGELPAPHDEPTEAFARQVPHVYEGGREFVSGVALAPSGASYYKLAALYQKARRCLH

SEQ ID NO: 38 - protein, 285 aa, Artificial
  APT73.77 (mutant soluble aromatic prenyltransferase)
MDEVYAAVEQTSRLLDVPCSPDRFEPVWKAFGDQLPDSHLVFSMAAGEAHRGELDFDFSLRPEGADPYTTALEHGFIEPTDHPVGSVLAEVGKRFAIASYGVEYGVVGGFKKSYAFFPLDDFPPLAQFAEVPSVPPCLAGHVETLTRLGFDDKVSIIGVNYRKNTLNVYLAASAVDTGDKLALLRAFGYPEPDARVRQFIERSFRLYPTFNWDSSAAERICFAVHTQQPGELPAPHDEPTEAFARQVPHVYEGGREFVSGVALAPSGASYYKLAALYQKARRCLD

SEQ ID NO: 39 - protein, 284 aa, Unknown
  APT89.38 (soluble aromatic prenyltransferase)
MDEVYAAVERTSRLLDVPCSPDRFEPVWKAFGDQLPDSHLVFSMAAGEAHRGELDFDFSLRPEGADPYTTALEHGFIEPTDHPVGSVLAEVNKRCEIASYGVEYGVVGGFKKSYAFFPLDDFPPLAEFARIPSVPPCLAGHVDTLTRLGLDDKVSAIGVNYRKNTLNVYLAASAVATDDKLALLRAFGYPEPDARVRQFIERSFRLYPTFNWDSSAAERICFAVHTQQPGELPAPHDEPTEAFAREVPHVYEGGREFVSGVALAPSGAAYYKLAAEYQKERRCL

SEQ ID NO: 40-51 - skipped (12 entries)

SEQ ID NO: 52 - protein, 309 aa, Artificial
  MPT4.1 (point mutant, membrane-bound prenyltransferase)
MSDNSIATKILNFGHTCWKLQRPYAVKGMISIACGLFGRELFNNRHLFSWGLMWKAFFALVPILSFNFFAAIMNQIYDVDIDRINKPDLPLVSGEMSIETAWILSIIVALTGLIVTIKLKSAPLFVFIYIFGIFAGFAYSVPPIRWKQYPFTNFLITISSHVGLAFTSYSATTSALGLPFVWRPAFSFIIAFMTVMGMTIAFAKDISDIEGDAKYGVSTVATKLGARNMTFVVSGVLLLNYLVSISIGIIWPQVFKSNIMILSHAILAFCLIFQTRELALANYASAPSRQFFEFIWLLYYAEYFVYVFI

SEQ ID NO: 53 - protein, 683 aa, Artificial; identical to SEQ ID NO: 18
  GPS1.1-F9-MPT4.1, geranyl diphosphate synthase, point mutant membrane-bound prenyltransferase fusion protein

SEQ ID NO: 54 - protein, 668 aa, Artificial
  GPS1.1-F5-MPT4.1, geranyl diphosphate synthase, point mutant membrane-bound prenyltransferase fusion protein
MSKAKFESVFPRISEELVQLLRDEGLPQDAVQWFSDSLQYNCVGGKLNRGLSVVDTYQLLTGKKELDDEEYYRLALLGWLIELLQAFWLVSDDIMDESKTRRGQPCWYLKPKVGMIAIWDAFMLESGIYILLKKHFRQEKYYIDLVELFHDISFKTELGQLVDLLTAPEDEVDLNRFSLDKHSFIVRYKTAYYSFYLPVVLAMYVAGITNPKDLQQAMDVLIPLGEYFQVQDDYLDNFGDPEFIGKIGTDIQDNKCSWLVNKALQKATPEQRQILEDNYGVKDKSKELVIKKLYDDMKIEQDYLDYEEEVVGDIKKKIEQVDESRGFKKEVLNAFLAKIYKRQKGGGGSGGGGSGGGGSMSDNSIATKILNFGHTCWKLQRPYAVKGMISIACGLFGRELFNNRHLFSWGLMWKAFFALVPILSFNFFAAIMNQIYDVDIDRINKPDLPLVSGEMSIETAWILSIIVALTGLIVTIKLKSAPLFVFIYIFGIFAGFAYSVPPIRWKQYPFTNFLITISSHVGLAFTSYSATTSALGLPFVWRPAFSFIIAFMTVMGMTIAFAKDISDIEGDAKYGVSTVATKLGARNMTFVVSGVLLLNYLVSISIGIIWPQVFKSNIMILSHAILAFCLIFQTRELALANYASAPSRQFFEFIWLLYYAEYFVYVFI

SEQ ID NO: 55 - protein, 705 aa, Artificial
  GPS1.1-F10-MPT4.1, geranyl diphosphate synthase, point mutant membrane-bound prenyltransferase fusion protein
MSKAKFESVFPRISEELVQLLRDEGLPQDAVQWFSDSLQYNCVGGKLNRGLSVVDTYQLLTGKKELDDEEYYRLALLGWLIELLQAFWLVSDDIMDESKTRRGQPCWYLKPKVGMIAIWDAFMLESGIYILLKKHFRQEKYYIDLVELFHDISFKTELGQLVDLLTAPEDEVDLNRFSLDKHSFIVRYKTAYYSFYLPVVLAMYVAGITNPKDLQQAMDVLIPLGEYFQVQDDYLDNFGDPEFIGKIGTDIQDNKCSWLVNKALQKATPEQRQILEDNYGVKDKSKELVIKKLYDDMKIEQDYLDYEEEVVGDIKKKIEQVDESRGFKKEVLNAFLAKIYKRQKGGAEAAAKEAAAKAAEAAAKEAAAKAAEAAAKEAAAKAAEAAAKEAAAKAGGMSDNSIATKILNFGHTCWKLQRPYAVKGMISIACGLFGRELFNNRHLFSWGLMWKAFFALVPILSFNFFAAIMNQIYDVDIDRINKPDLPLVSGEMSIETAWILSIIVALTGLIVTIKLKSAPLFVFIYIFGIFAGFAYSVPPIRWKQYPFTNFLITISSHVGLAFTSYSATTSALGLPFVWRPAFSFIIAFMTVMGMTIAFAKDISDIEGDAKYGVSTVATKLGARNMTFVVSGVLLLNYLVSISIGIIWPQVFKSNIMILSHAILAFCLIFQTRELALANYASAPSRQFFEFIWLLYYAEYFVYVFI

SEQ ID NO: 56 - protein, 692 aa, Artificial
  GPS1.1-F11-MPT4.1, geranyl diphosphate synthase, point mutant membrane-bound prenyltransferase fusion protein
MSKAKFESVFPRISEELVQLLRDEGLPQDAVQWFSDSLQYNCVGGKLNRGLSVVDTYQLLTGKKELDDEEYYRLALLGWLIELLQAFWLVSDDIMDESKTRRGQPCWYLKPKVGMIAIWDAFMLESGIYILLKKHFRQEKYYIDLVELFHDISFKTELGQLVDLLTAPEDEVDLNRFSLDKHSFIVRYKTAYYSFYLPVVLAMYVAGITNPKDLQQAMDVLIPLGEYFQVQDDYLDNFGDPEFIGKIGTDIQDNKCSWLVNKALQKATPEQRQILEDNYGVKDKSKELVIKKLYDDMKIEQDYLDYEEEVVGDIKKKIEQVDESRGFKKEVLNAFLAKIYKRQKGGAEAAAKEAAAKAGGSGGGSGGGGSGGSGGGGSGGGGSMSDNSIATKILNFGHTCWKLQRPYAVKGMISIACGLFGRELFNNRHLFSWGLMWKAFFALVPILSFNFFAAIMNQIYDVDIDRINKPDLPLVSGEMSIETAWILSIIVALTGLIVTIKLKSAPLFVFIYIFGIFAGFAYSVPPIRWKQYPFTNFLITISSHVGLAFTSYSATTSALGLPFVWRPAFSFIIAFMTVMGMTIAFAKDISDIEGDAKYGVSTVATKLGARNMTFVVSGVLLLNYLVSISIGIIWPQVFKSNIMILSHAILAFCLIFQTRELALANYASAPSRQFFEFIWLLYYAEYFVYVFI

SEQ ID NO: 57 - protein, 658 aa, Artificial
  GPS3-F11-MPT4.1, geranyl diphosphate synthase, point mutant membrane-bound prenyltransferase fusion protein
MQLLNPPQKGKKAVEFDFNKYMDSKAMTVNEALNKAIPLRYPQKIYESMRYSLLAGGKRVRPVLCIAACELVGGTEELAIPTACAIEMIHTMSLMHDDLPCIDNDDLRRGKPTNHKIFGEDTAVTAGNALHSYAFEHIAVSTSKTVGADRILRMVSELGRATGSEGVMGGQMVDIASEGDPSIDLQTLEWIHIHKTAMLLECSVVCGAIIGGASEIVIERARRYARCVGLLFQVVDDILDVTKSSDELGKTAGKDLISDKATYPKLMGLEKAKEFSDELLNRAKGELSCFDPVKAAPLLGLADYVAFRQNGGAEAAAKEAAAKAGGSGGGSGGGGSGGSGGGGSGGGGSMSDNSIATKILNFGHTCWKLQRPYAVKGMISIACGLFGRELFNNRHLFSWGLMWKAFFALVPILSFNFFAAIMNQIYDVDIDRINKPDLPLVSGEMSIETAWILSIIVALTGLIVTIKLKSAPLFVFIYIFGIFAGFAYSVPPIRWKQYPFTNFLITISSHVGLAFTSYSATTSALGLPFVWRPAFSFIIAFMTVMGMTIAFAKDISDIEGDAKYGVSTVATKLGARNMTFVVSGVLLLNYLVSISIGIIWPQVFKSNIMILSHAILAFCLIFQTRELALANYASAPSRQFFEFIWLLYYAEYFVYVFI

SEQ ID NO: 58 - protein, 719 aa, Artificial
  GPS2-F11-MPT4.1, geranyl diphosphate synthase, point mutant membrane-bound prenyltransferase fusion protein
MKDVSLSSFDAHDLDLDKFPEVVRDRLTQFLDAQELTIADIGAPVTDAVAHLRSFVLNGGKRIRPLYAWAGFLAAQGHKNSSEKLESVLDAAASLEFIQACALIHDDIIDSSDTRRGAPTVHRAVEADHRANNFEGDPEHFGVSVSILAGDMALVWAEDMLQDSGLSAEALARTRDAWRGMRTEVIGGQLLDIYLESHANESVELADSVNRFKTAAYTIARPLHLGASIAGGSPQLIDALLHYGHDIGIAFQLRDDLLGVFGDPAITGKPAGDDIREGKRTVLLALALQRADKQSPEAATAIRAGVGKVTSPEDIAVITEHIRATGAEEEVEQRISQLTESGLAHLDDVDIPDEVRAQLRALAIRSTERRMGGAEAAAKEAAAKAGGSGGGSGGGGSGGSGGGGSGGGGSMSDNSIATKILNFGHTCWKLQRPYAVKGMISIACGLFGRELFNNRHLFSWGLMWKAFFALVPILSFNFFAAIMNQIYDVDIDRINKPDLPLVSGEMSIETAWILSIIVALTGLIVTIKLKSAPLFVFIYIFGIFAGFAYSVPPIRWKQYPFTNFLITISSHVGLAFTSYSATTSALGLPFVWRPAFSFIIAFMTVMGMTIAFAKDISDIEGDAKYGVSTVATKLGARNMTFVVSGVLLLNYLVSISIGIIWPQVFKSNIMILSHAILAFCLIFQTRELALANYASAPSRQFFEFIWLLYYAEYFVYVFI

SEQ ID NO: 59 - protein, 646 aa, Artificial
  GPS1.1-F16-APT73.74, geranyl diphosphate synthase, mutant soluble aromatic prenyltransferase fusion protein
MSKAKFESVFPRISEELVQLLRDEGLPQDAVQWFSDSLQYNCVGGKLNRGLSVVDTYQLLTGKKELDDEEYYRLALLGWLIELLQAFWLVSDDIMDESKTRRGQPCWYLKPKVGMIAIWDAFMLESGIYILLKKHFRQEKYYIDLVELFHDISFKTELGQLVDLLTAPEDEVDLNRFSLDKHSFIVRYKTAYYSFYLPVVLAMYVAGITNPKDLQQAMDVLIPLGEYFQVQDDYLDNFGDPEFIGKIGTDIQDNKCSWLVNKALQKATPEQRQILEDNYGVKDKSKELVIKKLYDDMKIEQDYLDYEEEVVGDIKKKIEQVDESRGFKKEVLNAFLAKIYKRQKGGGSGGGSGGGSGGGGSMDEVYAAVEQTSRLLDVPCSPDRFEPVWKAFGDQLPDSHLVFSMAAGEAHRGELDFDFSLRPEGADPYTTALEHGFIEPTDHPVGSVLAEVGKRFAIASYGVEYGVVGGFKKSYAFFPLDDFPPLAQFAEVPSVPPCLAGHVETLTRLGFDDKVSIIGVNYRKNTLNVYLAASAVDTGDKLALLRAFGYPEPDARVRQFIERSFRLYPTFNWDSSAAERICFAVHTQQPGELPAPHDEPTEAFARQVPHVYEGGREFVSGVALAPSGASYYKLAALYQKARRCLH

SEQ ID NO: 60 - protein, 634 aa, Artificial
  APT73.74-F17-GPS1.1, mutant soluble aromatic prenyltransferase, geranyl diphosphate synthase fusion protein
MDEVYAAVEQTSRLLDVPCSPDRFEPVWKAFGDQLPDSHLVFSMAAGEAHRGELDFDFSLRPEGADPYTTALEHGFIEPTDHPVGSVLAEVGKRFAIASYGVEYGVVGGFKKSYAFFPLDDFPPLAQFAEVPSVPPCLAGHVETLTRLGFDDKVSIIGVNYRKNTLNVYLAASAVDTGDKLALLRAFGYPEPDARVRQFIERSFRLYPTFNWDSSAAERICFAVHTQQPGELPAPHDEPTEAFARQVPHVYEGGREFVSGVALAPSGASYYKLAALYQKARRCLHGGGGSMSKAKFESVFPRISEELVQLLRDEGLPQDAVQWFSDSLQYNCVGGKLNRGLSVVDTYQLLTGKKELDDEEYYRLALLGWLIELLQAFWLVSDDIMDESKTRRGQPCWYLKPKVGMIAIWDAFMLESGIYILLKKHFRQEKYYIDLVELFHDISFKTELGQLVDLLTAPEDEVDLNRFSLDKHSFIVRYKTAYYSFYLPVVLAMYVAGITNPKDLQQAMDVLIPLGEYFQVQDDYLDNFGDPEFIGKIGTDIQDNKCSWLVNKALQKATPEQRQILEDNYGVKDKSKELVIKKLYDDMKIEQDYLDYEEEVVGDIKKKIEQVDESRGFKKEVLNAFLAKIYKRQK

SEQ ID NO: 61 - protein, 677 aa, Artificial
  GPS1.1-F18-APT73.74, geranyl diphosphate synthase, mutant soluble aromatic prenyltransferase fusion protein
MSKAKFESVFPRISEELVQLLRDEGLPQDAVQWFSDSLQYNCVGGKLNRGLSVVDTYQLLTGKKELDDEEYYRLALLGWLIELLQAFWLVSDDIMDESKTRRGQPCWYLKPKVGMIAIWDAFMLESGIYILLKKHFRQEKYYIDLVELFHDISFKTELGQLVDLLTAPEDEVDLNRFSLDKHSFIVRYKTAYYSFYLPVVLAMYVAGITNPKDLQQAMDVLIPLGEYFQVQDDYLDNFGDPEFIGKIGTDIQDNKCSWLVNKALQKATPEQRQILEDNYGVKDKSKELVIKKLYDDMKIEQDYLDYEEEVVGDIKKKIEQVDESRGFKKEVLNAFLAKIYKRQKGGGGSLEDPAVWEAGKVVAKGVGTADITATTSNGLIASSEEADNAATSMDEVYAAVEQTSRLLDVPCSPDRFEPVWKAFGDQLPDSHLVFSMAAGEAHRGELDFDFSLRPEGADPYTTALEHGFIEPTDHPVGSVLAEVGKRFAIASYGVEYGVVGGFKKSYAFFPLDDFPPLAQFAEVPSVPPCLAGHVETLTRLGFDDKVSIIGVNYRKNTLNVYLAASAVDTGDKLALLRAFGYPEPDARVRQFIERSFRLYPTFNWDSSAAERICFAVHTQQPGELPAPHDEPTEAFARQVPHVYEGGREFVSGVALAPSGASYYKLAALYQKARRCLH

SEQ ID NO: 62 - protein, 677 aa, Artificial
  APT73.74-F18-GPS1.1, mutant soluble aromatic prenyltransferase, geranyl diphosphate synthase fusion protein
MDEVYAAVEQTSRLLDVPCSPDRFEPVWKAFGDQLPDSHLVFSMAAGEAHRGELDFDFSLRPEGADPYTTALEHGFIEPTDHPVGSVLAEVGKRFAIASYGVEYGVVGGFKKSYAFFPLDDFPPLAQFAEVPSVPPCLAGHVETLTRLGFDDKVSIIGVNYRKNTLNVYLAASAVDTGDKLALLRAFGYPEPDARVRQFIERSFRLYPTFNWDSSAAERICFAVHTQQPGELPAPHDEPTEAFARQVPHVYEGGREFVSGVALAPSGASYYKLAALYQKARRCLHGGGGSLEDPAVWEAGKVVAKGVGTADITATTSNGLIASSEEADNAATSMSKAKFESVFPRISEELVQLLRDEGLPQDAVQWFSDSLQYNCVGGKLNRGLSVVDTYQLLTGKKELDDEEYYRLALLGWLIELLQAFWLVSDDIMDESKTRRGQPCWYLKPKVGMIAIWDAFMLESGIYILLKKHFRQEKYYIDLVELFHDISFKTELGQLVDLLTAPEDEVDLNRFSLDKHSFIVRYKTAYYSFYLPVVLAMYVAGITNPKDLQQAMDVLIPLGEYFQVQDDYLDNFGDPEFIGKIGTDIQDNKCSWLVNKALQKATPEQRQILEDNYGVKDKSKELVIKKLYDDMKIEQDYLDYEEEVVGDIKKKIEQVDESRGFKKEVLNAFLAKIYKRQK

SEQ ID NO: 63 - protein, 646 aa, Artificial
  GPS1.1-F16-APT73.77, geranyl diphosphate synthase, mutant soluble aromatic prenyltransferase fusion protein
MSKAKFESVFPRISEELVQLLRDEGLPQDAVQWFSDSLQYNCVGGKLNRGLSVVDTYQLLTGKKELDDEEYYRLALLGWLIELLQAFWLVSDDIMDESKTRRGQPCWYLKPKVGMIAIWDAFMLESGIYILLKKHFRQEKYYIDLVELFHDISFKTELGQLVDLLTAPEDEVDLNRFSLDKHSFIVRYKTAYYSFYLPVVLAMYVAGITNPKDLQQAMDVLIPLGEYFQVQDDYLDNFGDPEFIGKIGTDIQDNKCSWLVNKALQKATPEQRQILEDNYGVKDKSKELVIKKLYDDMKIEQDYLDYEEEVVGDIKKKIEQVDESRGFKKEVLNAFLAKIYKRQKGGGSGGGSGGGSGGGGSMDEVYAAVEQTSRLLDVPCSPDRFEPVWKAFGDQLPDSHLVFSMAAGEAHRGELDFDFSLRPEGADPYTTALEHGFIEPTDHPVGSVLAEVGKRFAIASYGVEYGVVGGFKKSYAFFPLDDFPPLAQFAEVPSVPPCLAGHVETLTRLGFDDKVSIIGVNYRKNTLNVYLAASAVDTGDKLALLRAFGYPEPDARVRQFIERSFRLYPTFNWDSSAAERICFAVHTQQPGELPAPHDEPTEAFARQVPHVYEGGREFVSGVALAPSGASYYKLAALYQKARRCLD

SEQ ID NO: 64 - protein, 634 aa, Artificial
  APT73.77-F17-GPS1.1, mutant soluble aromatic prenyltransferase, geranyl diphosphate synthase fusion protein
MDEVYAAVEQTSRLLDVPCSPDRFEPVWKAFGDQLPDSHLVFSMAAGEAHRGELDFDFSLRPEGADPYTTALEHGFIEPTDHPVGSVLAEVGKRFAIASYGVEYGVVGGFKKSYAFFPLDDFPPLAQFAEVPSVPPCLAGHVETLTRLGFDDKVSIIGVNYRKNTLNVYLAASAVDTGDKLALLRAFGYPEPDARVRQFIERSFRLYPTFNWDSSAAERICFAVHTQQPGELPAPHDEPTEAFARQVPHVYEGGREFVSGVALAPSGASYYKLAALYQKARRCLDGGGGSMSKAKFESVFPRISEELVQLLRDEGLPQDAVQWFSDSLQYNCVGGKLNRGLSVVDTYQLLTGKKELDDEEYYRLALLGWLIELLQAFWLVSDDIMDESKTRRGQPCWYLKPKVGMIAIWDAFMLESGIYILLKKHFRQEKYYIDLVELFHDISFKTELGQLVDLLTAPEDEVDLNRFSLDKHSFIVRYKTAYYSFYLPVVLAMYVAGITNPKDLQQAMDVLIPLGEYFQVQDDYLDNFGDPEFIGKIGTDIQDNKCSWLVNKALQKATPEQRQILEDNYGVKDKSKELVIKKLYDDMKIEQDYLDYEEEVVGDIKKKIEQVDESRGFKKEVLNAFLAKIYKRQK

SEQ ID NO: 65 - protein, 677 aa, Artificial
  GPS1.1-F18-APT73.77, geranyl diphosphate synthase, mutant soluble aromatic prenyltransferase fusion protein
MSKAKFESVFPRISEELVQLLRDEGLPQDAVQWFSDSLQYNCVGGKLNRGLSVVDTYQLLTGKKELDDEEYYRLALLGWLIELLQAFWLVSDDIMDESKTRRGQPCWYLKPKVGMIAIWDAFMLESGIYILLKKHFRQEKYYIDLVELFHDISFKTELGQLVDLLTAPEDEVDLNRFSLDKHSFIVRYKTAYYSFYLPVVLAMYVAGITNPKDLQQAMDVLIPLGEYFQVQDDYLDNFGDPEFIGKIGTDIQDNKCSWLVNKALQKATPEQRQILEDNYGVKDKSKELVIKKLYDDMKIEQDYLDYEEEVVGDIKKKIEQVDESRGFKKEVLNAFLAKIYKRQKGGGGSLEDPAVWEAGKVVAKGVGTADITATTSNGLIASSEEADNAATSMDEVYAAVEQTSRLLDVPCSPDRFEPVWKAFGDQLPDSHLVFSMAAGEAHRGELDFDFSLRPEGADPYTTALEHGFIEPTDHPVGSVLAEVGKRFAIASYGVEYGVVGGFKKSYAFFPLDDFPPLAQFAEVPSVPPCLAGHVETLTRLGFDDKVSIIGVNYRKNTLNVYLAASAVDTGDKLALLRAFGYPEPDARVRQFIERSFRLYPTFNWDSSAAERICFAVHTQQPGELPAPHDEPTEAFARQVPHVYEGGREFVSGVALAPSGASYYKLAALYQKARRCLD

SEQ ID NO: 66 - protein, 677 aa, Artificial
  APT73.77-F18-GPS1.1, mutant soluble aromatic prenyltransferase, geranyl diphosphate synthase fusion protein
MDEVYAAVEQTSRLLDVPCSPDRFEPVWKAFGDQLPDSHLVFSMAAGEAHRGELDFDFSLRPEGADPYTTALEHGFIEPTDHPVGSVLAEVGKRFAIASYGVEYGVVGGFKKSYAFFPLDDFPPLAQFAEVPSVPPCLAGHVETLTRLGFDDKVSIIGVNYRKNTLNVYLAASAVDTGDKLALLRAFGYPEPDARVRQFIERSFRLYPTFNWDSSAAERICFAVHTQQPGELPAPHDEPTEAFARQVPHVYEGGREFVSGVALAPSGASYYKLAALYQKARRCLDGGGGSLEDPAVWEAGKVVAKGVGTADITATTSNGLIASSEEADNAATSMSKAKFESVFPRISEELVQLLRDEGLPQDAVQWFSDSLQYNCVGGKLNRGLSVVDTYQLLTGKKELDDEEYYRLALLGWLIELLQAFWLVSDDIMDESKTRRGQPCWYLKPKVGMIAIWDAFMLESGIYILLKKHFRQEKYYIDLVELFHDISFKTELGQLVDLLTAPEDEVDLNRFSLDKHSFIVRYKTAYYSFYLPVVLAMYVAGITNPKDLQQAMDVLIPLGEYFQVQDDYLDNFGDPEFIGKIGTDIQDNKCSWLVNKALQKATPEQRQILEDNYGVKDKSKELVIKKLYDDMKIEQDYLDYEEEVVGDIKKKIEQVDESRGFKKEVLNAFLAKIYKRQK

SEQ ID NO: 67 - protein, 645 aa, Artificial
  GPS1.1-F16-APT89.38, geranyl diphosphate synthase, soluble aromatic prenyltransferase fusion protein
MSKAKFESVFPRISEELVQLLRDEGLPQDAVQWFSDSLQYNCVGGKLNRGLSVVDTYQLLTGKKELDDEEYYRLALLGWLIELLQAFWLVSDDIMDESKTRRGQPCWYLKPKVGMIAIWDAFMLESGIYILLKKHFRQEKYYIDLVELFHDISFKTELGQLVDLLTAPEDEVDLNRFSLDKHSFIVRYKTAYYSFYLPVVLAMYVAGITNPKDLQQAMDVLIPLGEYFQVQDDYLDNFGDPEFIGKIGTDIQDNKCSWLVNKALQKATPEQRQILEDNYGVKDKSKELVIKKLYDDMKIEQDYLDYEEEVVGDIKKKIEQVDESRGFKKEVLNAFLAKIYKRQKGGGSGGGSGGGSGGGGSMDEVYAAVERTSRLLDVPCSPDRFEPVWKAFGDQLPDSHLVFSMAAGEAHRGELDFDFSLRPEGADPYTTALEHGFIEPTDHPVGSVLAEVNKRCEIASYGVEYGVVGGFKKSYAFFPLDDFPPLAEFARIPSVPPCLAGHVDTLTRLGLDDKVSAIGVNYRKNTLNVYLAASAVATDDKLALLRAFGYPEPDARVRQFIERSFRLYPTFNWDSSAAERICFAVHTQQPGELPAPHDEPTEAFAREVPHVYEGGREFVSGVALAPSGAAYYKLAAEYQKERRCL

SEQ ID NO: 68 - protein, 633 aa, Artificial
  APT89.38-F17-GPS1.1, soluble aromatic prenyltransferase, geranyl diphosphate synthase fusion protein
MDEVYAAVERTSRLLDVPCSPDRFEPVWKAFGDQLPDSHLVFSMAAGEAHRGELDFDFSLRPEGADPYTTALEHGFIEPTDHPVGSVLAEVNKRCEIASYGVEYGVVGGFKKSYAFFPLDDFPPLAEFARIPSVPPCLAGHVDTLTRLGLDDKVSAIGVNYRKNTLNVYLAASAVATDDKLALLRAFGYPEPDARVRQFIERSFRLYPTFNWDSSAAERICFAVHTQQPGELPAPHDEPTEAFAREVPHVYEGGREFVSGVALAPSGAAYYKLAAEYQKERRCLGGGGSMSKAKFESVFPRISEELVQLLRDEGLPQDAVQWFSDSLQYNCVGGKLNRGLSVVDTYQLLTGKKELDDEEYYRLALLGWLIELLQAFWLVSDDIMDESKTRRGQPCWYLKPKVGMIAIWDAFMLESGIYILLKKHFRQEKYYIDLVELFHDISFKTELGQLVDLLTAPEDEVDLNRFSLDKHSFIVRYKTAYYSFYLPVVLAMYVAGITNPKDLQQAMDVLIPLGEYFQVQDDYLDNFGDPEFIGKIGTDIQDNKCSWLVNKALQKATPEQRQILEDNYGVKDKSKELVIKKLYDDMKIEQDYLDYEEEVVGDIKKKIEQVDESRGFKKEVLNAFLAKIYKRQK

SEQ ID NO: 69 - protein, 676 aa, Artificial
  GPS1.1-F18-APT89.38, geranyl diphosphate synthase, soluble aromatic prenyltransferase fusion protein
MSKAKFESVFPRISEELVQLLRDEGLPQDAVQWFSDSLQYNCVGGKLNRGLSVVDTYQLLTGKKELDDEEYYRLALLGWLIELLQAFWLVSDDIMDESKTRRGQPCWYLKPKVGMIAIWDAFMLESGIYILLKKHFRQEKYYIDLVELFHDISFKTELGQLVDLLTAPEDEVDLNRFSLDKHSFIVRYKTAYYSFYLPVVLAMYVAGITNPKDLQQAMDVLIPLGEYFQVQDDYLDNFGDPEFIGKIGTDIQDNKCSWLVNKALQKATPEQRQILEDNYGVKDKSKELVIKKLYDDMKIEQDYLDYEEEVVGDIKKKIEQVDESRGFKKEVLNAFLAKIYKRQKGGGGSLEDPAVWEAGKVVAKGVGTADITATTSNGLIASSEEADNAATSMDEVYAAVERTSRLLDVPCSPDRFEPVWKAFGDQLPDSHLVFSMAAGEAHRGELDFDFSLRPEGADPYTTALEHGFIEPTDHPVGSVLAEVNKRCEIASYGVEYGVVGGFKKSYAFFPLDDFPPLAEFARIPSVPPCLAGHVDTLTRLGLDDKVSAIGVNYRKNTLNVYLAASAVATDDKLALLRAFGYPEPDARVRQFIERSFRLYPTFNWDSSAAERICFAVHTQQPGELPAPHDEPTEAFAREVPHVYEGGREFVSGVALAPSGAAYYKLAAEYQKERRCL

SEQ ID NO: 70 - protein, 676 aa, Artificial
  APT89.38-F18-GPS1.1, soluble aromatic prenyltransferase, geranyl diphosphate synthase fusion protein
MDEVYAAVERTSRLLDVPCSPDRFEPVWKAFGDQLPDSHLVFSMAAGEAHRGELDFDFSLRPEGADPYTTALEHGFIEPTDHPVGSVLAEVNKRCEIASYGVEYGVVGGFKKSYAFFPLDDFPPLAEFARIPSVPPCLAGHVDTLTRLGLDDKVSAIGVNYRKNTLNVYLAASAVATDDKLALLRAFGYPEPDARVRQFIERSFRLYPTFNWDSSAAERICFAVHTQQPGELPAPHDEPTEAFAREVPHVYEGGREFVSGVALAPSGAAYYKLAAEYQKERRCLGGGGSLEDPAVWEAGKVVAKGVGTADITATTSNGLIASSEEADNAATSMSKAKFESVFPRISEELVQLLRDEGLPQDAVQWFSDSLQYNCVGGKLNRGLSVVDTYQLLTGKKELDDEEYYRLALLGWLIELLQAFWLVSDDIMDESKTRRGQPCWYLKPKVGMIAIWDAFMLESGIYILLKKHFRQEKYYIDLVELFHDISFKTELGQLVDLLTAPEDEVDLNRFSLDKHSFIVRYKTAYYSFYLPVVLAMYVAGITNPKDLQQAMDVLIPLGEYFQVQDDYLDNFGDPEFIGKIGTDIQDNKCSWLVNKALQKATPEQRQILEDNYGVKDKSKELVIKKLYDDMKIEQDYLDYEEEVVGDIKKKIEQVDESRGFKKEVLNAFLAKIYKRQK

SEQ ID NO: 71 - protein, 309 aa, Artificial
  MPT21.1 (recombinant membrane-bound prenyltransferase)
MSDNSIATKILNFGHTCWKLQRPFVVKGMISIACGLFGKELLHNTNLISWGLMWKAFFALVPILSFNFFAAIMNQIYDVDIDRINKPDLPLVSGEMSIETAWILSIIVALTGLIVTIKLKSAPLFVFIYIFGIFAGFAYSVPPIRWKQNPSTNFLITISSHVGLAFTSYYASRAALGLPFELRPSFTFLLAFMTVMGMTIAFAKDISDIEGDAKYGVSTVATKLGARNMTFVVSGVLLLNYVAAILAGIIWPQAFNSNVMLLSHAILAFCLIFQTRELALANYASAPSRQFFEFIWLLYYAEYFVYVFI

SEQ ID NO: 72 - protein, 309 aa, Artificial
  MPT21.2 (recombinant membrane-bound prenyltransferase)
MSDNSIATKILNFGHTCWKLQRPMVVKGMISIACGLFGKELLHNTNLISWGLMWKAFFALVPILSFNFFAAIMNQIYDVDIDRINKPDLPLVSGEMSIETAWILSIIVALTGLIVTIKLKSAPLFVFIYIFGIFAGFAYSVPPIRWKQNPSTNFLITISSHVGLAFTSYYASRAALGLPFELRPSFTFLLAFMTVMGMTIAFAKDISDIEGDAKYGVSTVATKLGARNMTFVVSGVLLLNYVAAILAGIIWPQAFNSNVMLLSHAILAFCLIFQTRELALANYASAPSRQFFEFIWLLYYAEYFVYVFI

SEQ ID NO: 73 - protein, 309 aa, Artificial
  MPT21.3 (recombinant membrane-bound prenyltransferase)
MSDNSIATKILNFGHTCWKLQRPYAVKGMISIACGLFGKELLHNTNLISWGLMWKAFFALVPILSFNFFAAIMNQIYDVDIDRINKPDLPLVSGEMSIETAWILSIIVALTGLIVTIKLKSAPLFVFIYIFGIFAGFAYSVPPIRWKQNPSTNFLITISSHVGLAFTSYYASRAALGLPFELRPSFTFLLAFMTVMGMTIAFAKDISDIEGDAKYGVSTVATKLGARNMTFVVSGVLLLNYVAAILAGIIWPQAFNSNVMLLSHAILAFCLIFQTRELALANYASAPSRQFFEFIWLLYYAEYFVYVFI

SEQ ID NO: 74 - protein, 309 aa, Artificial
  MPT21.4 (recombinant membrane-bound prenyltransferase)
MSDNSIATKILNFGHTCWKLQRPYVVKGAISIACGLFGKELLHNTNLISWGLMWKAFFALVPILSFNFFAAIMNQIYDVDIDRINKPDLPLVSGEMSIETAWILSIIVALTGLIVTIKLKSAPLFVFIYIFGIFAGFAYSVPPIRWKQNPSTNFLITISSHVGLAFTSYYASRAALGLPFELRPSFTFLLAFMTVMGMTIAFAKDISDIEGDAKYGVSTVATKLGARNMTFVVSGVLLLNYVAAILAGIIWPQAFNSNVMLLSHAILAFCLIFQTRELALANYASAPSRQFFEFIWLLYYAEYFVYVFI

SEQ ID NO: 75 - protein, 309 aa, Artificial
  MPT21.5 (recombinant membrane-bound prenyltransferase)
MSDNSIATKILNFGHTCWKLQRPYVVKGMITIACGLFGKELLHNTNLISWGLMWKAFFALVPILSFNFFAAIMNQIYDVDIDRINKPDLPLVSGEMSIETAWILSIIVALTGLIVTIKLKSAPLFVFIYIFGIFAGFAYSVPPIRWKQNPSTNFLITISSHVGLAFTSYYASRAALGLPFELRPSFTFLLAFMTVMGMTIAFAKDISDIEGDAKYGVSTVATKLGARNMTFVVSGVLLLNYVAAILAGIIWPQAFNSNVMLLSHAILAFCLIFQTRELALANYASAPSRQFFEFIWLLYYAEYFVYVFI

SEQ ID NO: 76 - protein, 309 aa, Artificial
  MPT21.6 (recombinant membrane-bound prenyltransferase)
MSDNSIATKILNFGHTCWKLQRPYVVKGMIVIACGLFGKELLHNTNLISWGLMWKAFFALVPILSFNFFAAIMNQIYDVDIDRINKPDLPLVSGEMSIETAWILSIIVALTGLIVTIKLKSAPLFVFIYIFGIFAGFAYSVPPIRWKQNPSTNFLITISSHVGLAFTSYYASRAALGLPFELRPSFTFLLAFMTVMGMTIAFAKDISDIEGDAKYGVSTVATKLGARNMTFVVSGVLLLNYVAAILAGIIWPQAFNSNVMLLSHAILAFCLIFQTRELALANYASAPSRQFFEFIWLLYYAEYFVYVFI

SEQ ID NO: 77 - protein, 309 aa, Artificial
  MPT21.7 (recombinant membrane-bound prenyltransferase)
MSDNSIATKILNFGHTCWKLQRPYVVKGMIAIACGLFGKELLHNTNLISWGLMWKAFFALVPILSFNFFAAIMNQIYDVDIDRINKPDLPLVSGEMSIETAWILSIIVALTGLIVTIKLKSAPLFVFIYIFGIFAGFAYSVPPIRWKQNPSTNFLITISSHVGLAFTSYYASRAALGLPFELRPSFTFLLAFMTVMGMTIAFAKDISDIEGDAKYGVSTVATKLGARNMTFVVSGVLLLNYVAAILAGIIWPQAFNSNVMLLSHAILAFCLIFQTRELALANYASAPSRQFFEFIWLLYYAEYFVYVFI

SEQ ID NO: 78 - protein, 309 aa, Artificial
  MPT21.8 (recombinant membrane-bound prenyltransferase)
MSDNSIATKILNFGHTCWKLQRPYVVKGMISIACGLFGKELLHNTNLISWGLMWKAFFALVPILSFNFFAGIMNQIYDVDIDRINKPDLPLVSGEMSIETAWILSIIVALTGLIVTIKLKSAPLFVFIYIFGIFAGFAYSVPPIRWKQNPSTNFLITISSHVGLAFTSYYASRAALGLPFELRPSFTFLLAFMTVMGMTIAFAKDISDIEGDAKYGVSTVATKLGARNMTFVVSGVLLLNYVAAILAGIIWPQAFNSNVMLLSHAILAFCLIFQTRELALANYASAPSRQFFEFIWLLYYAEYFVYVFI

SEQ ID NO: 79 - protein, 309 aa, Artificial
  MPT21.9 (recombinant membrane-bound prenyltransferase)
MSDNSIATKILNFGHTCWKLQRPYVVKGMISIACGLFGKELLHNTNLISWGLMWKAFFALVPILSFNFFASIMNQIYDVDIDRINKPDLPLVSGEMSIETAWILSIIVALTGLIVTIKLKSAPLFVFIYIFGIFAGFAYSVPPIRWKQNPSTNFLITISSHVGLAFTSYYASRAALGLPFELRPSFTFLLAFMTVMGMTIAFAKDISDIEGDAKYGVSTVATKLGARNMTFVVSGVLLLNYVAAILAGIIWPQAFNSNVMLLSHAILAFCLIFQTRELALANYASAPSRQFFEFIWLLYYAEYFVYVFI

SEQ ID NO: 80 - protein, 309 aa, Artificial
  MPT21.10 (recombinant membrane-bound prenyltransferase)
MSDNSIATKILNFGHTCWKLQRPYVVKGMISIACGLFGKELLHNTNLISWGLMWKAFFALVPILSFNFFAAIMNQIYDVDMDRINKPDLPLVSGEMSIETAWILSIIVALTGLIVTIKLKSAPLFVFIYIFGIFAGFAYSVPPIRWKQNPSTNFLITISSHVGLAFTSYYASRAALGLPFELRPSFTFLLAFMTVMGMTIAFAKDISDIEGDAKYGVSTVATKLGARNMTFVVSGVLLLNYVAAILAGIIWPQAFNSNVMLLSHAILAFCLIFQTRELALANYASAPSRQFFEFIWLLYYAEYFVYVFI

SEQ ID NO: 81 - protein, 309 aa, Artificial
  MPT21.11 (recombinant membrane-bound prenyltransferase)
MSDNSIATKILNFGHTCWKLQRPYVVKGMISIACGLFGKELLHNTNLISWGLMWKAFFALVPILSFNFFAAIMNQIYDVDIDRVNKPDLPLVSGEMSIETAWILSIIVALTGLIVTIKLKSAPLFVFIYIFGIFAGFAYSVPPIRWKQNPSTNFLITISSHVGLAFTSYYASRAALGLPFELRPSFTFLLAFMTVMGMTIAFAKDISDIEGDAKYGVSTVATKLGARNMTFVVSGVLLLNYVAAILAGIIWPQAFNSNVMLLSHAILAFCLIFQTRELALANYASAPSRQFFEFIWLLYYAEYFVYVFI

SEQ ID NO: 82 - protein, 309 aa, Artificial
  MPT21.12 (recombinant membrane-bound prenyltransferase)
MSDNSIATKILNFGHTCWKLQRPYVVKGMISIACGLFGKELLHNTNLISWGLMWKAFFALVPILSFNFFAAIMNQIYDVDIDRINKPDLPLVSGEMSIETAWILSIIVALTGLIVTIKLKSAPLFVFIYIFGIFAGFAYSVPPIRWKQNPSTNFEITISSHVGLAFTSYYASRAALGLPFELRPSFTFLLAFMTVMGMTIAFAKDISDIEGDAKYGVSTVATKLGARNMTFVVSGVLLLNYVAAILAGIIWPQAFNSNVMLLSHAILAFCLIFQTRELALANYASAPSRQFFEFIWLLYYAEYFVYVFI

SEQ ID NO: 83 - protein, 309 aa, Artificial
  MPT21.13 (recombinant membrane-bound prenyltransferase)
MSDNSIATKILNFGHTCWKLQRPYVVKGMISIACGLFGKELLHNTNLISWGLMWKAFFALVPILSFNFFAAIMNQIYDVDIDRINKPDLPLVSGEMSIETAWILSIIVALTGLIVTIKLKSAPLFVFIYIFGIFAGFAYSVPPIRWKQNPSTNFLITIASHVGLAFTSYYASRAALGLPFELRPSFTFLLAFMTVMGMTIAFAKDISDIEGDAKYGVSTVATKLGARNMTFVVSGVLLLNYVAAILAGIIWPQAFNSNVMLLSHAILAFCLIFQTRELALANYASAPSRQFFEFIWLLYYAEYFVYVFI

SEQ ID NO: 84 - protein, 309 aa, Artificial
  MPT21.14 (recombinant membrane-bound prenyltransferase)
MSDNSIATKILNFGHTCWKLQRPYVVKGMISIACGLFGKELLHNTNLISWGLMWKAFFALVPILSFNFFAAIMNQIYDVDIDRINKPDLPLVSGEMSIETAWILSIIVALTGLIVTIKLKSAPLFVFIYIFGIFAGFAYSVPPIRWKQNPSTNFLITIGSHVGLAFTSYYASRAALGLPFELRPSFTFLLAFMTVMGMTIAFAKDISDIEGDAKYGVSTVATKLGARNMTFVVSGVLLLNYVAAILAGIIWPQAFNSNVMLLSHAILAFCLIFQTRELALANYASAPSRQFFEFIWLLYYAEYFVYVFI

SEQ ID NO: 85 - protein, 309 aa, Artificial
  MPT21.15 (recombinant membrane-bound prenyltransferase)
MSDNSIATKILNFGHTCWKLQRPYVVKGMISIACGLFGKELLHNTNLISWGLMWKAFFALVPILSFNFFAAIMNQIYDVDIDRINKPDLPLVSGEMSIETAWILSIIVALTGLIVTIKLKSAPLFVFIYIFGIFAGFAYSVPPIRWKQNPSTNFLITIVSHVGLAFTSYYASRAALGLPFELRPSFTFLLAFMTVMGMTIAFAKDISDIEGDAKYGVSTVATKLGARNMTFVVSGVLLLNYVAAILAGIIWPQAFNSNVMLLSHAILAFCLIFQTRELALANYASAPSRQFFEFIWLLYYAEYFVYVFI

SEQ ID NO: 86 - protein, 309 aa, Artificial
  MPT21.16 (recombinant membrane-bound prenyltransferase)
MSDNSIATKILNFGHTCWKLQRPYVVKGMISIACGLFGKELLHNTNLISWGLMWKAFFALVPILSFNFFAAIMNQIYDVDIDRINKPDLPLVSGEMSIETAWILSIIVALTGLIVTIKLKSAPLFVFIYIFGIFAGFAYSVPPIRWKQNPSTNFLITISSHVGLAFTSYYASRAALGLPFELRPSFTFLLAFMTVMGMTIGFAKDISDIEGDAKYGVSTVATKLGARNMTFVVSGVLLLNYVAAILAGIIWPQAFNSNVMLLSHAILAFCLIFQTRELALANYASAPSRQFFEFIWLLYYAEYFVYVFI

SEQ ID NO: 87 - protein, 309 aa, Artificial
  MPT21.17 (recombinant membrane-bound prenyltransferase)
MSDNSIATKILNFGHTCWKLQRPYVVKGMISIACGLFGKELLHNTNLISWGLMWKAFFALVPILSFNFFAAIMNQIYDVDIDRINKPDLPLVSGEMSIETAWILSIIVALTGLIVTIKLKSAPLFVFIYIFGIFAGFAYSVPPIRWKQNPSTNFLITISSHVGLAFTSYYASRAALGLPFELRPSFTFLLAFMTVMGMTIAFAKDISDIEGDAKYGVSTVATKLGARNMTFVVSGVLLLNYVAAILAGIIWPQAFNSNVMLLSHAILAFCLIFQQRELALANYASAPSRQFFEFIWLLYYAEYFVYVFI

SEQ ID NO: 88 - protein, 309 aa, Artificial
  MPT21.18 (recombinant membrane-bound prenyltransferase)
MSDNSIATKILNFGHTCWKLQRPYVVKGMISIACGLFGKELLHNTNLISWGLMWKAFFALVPILSFNFFAAIMNQIYDVDIDRINKPDLPLVSGEMSIETAWILSIIVALTGLIVTIKLKSAPLFVFIYIFGIFAGFAYSVPPIRWKQNPSTNFLITISSHVGLAFTSYYASRAALGLPFELRPSFTFLLAFMTVMGMTIAFAKDISDIEGDAKYGVSTVATKLGARNMTFVVSGVLLLNYVAAILAGIIWPQAFNSNVMLLSHAILAFCLIFQARELALANYASAPSRQFFEFIWLLYYAEYFVYVFI

SEQ ID NO: 89 - protein, 309 aa, Artificial
  MPT21.19 (recombinant membrane-bound prenyltransferase)
MSDNSIATKILNFGHTCWKLQRPYVVKGMISIACGLFGKELLHNTNLISWGLMWKAFFALVPILSFNFFAAIMNQIYDVDIDRINKPDLPLVSGEMSIETAWILSIIVALTGLIVTIKLKSAPLFVFIYIFGIFAGFAYSVPPIRWKQNPSTNFLITISSHVGLAFTSYYASRAALGLPFELRPSFTFLLAFMTVMGMTIAFAKDISDIEGDAKYGVSTVATKLGARNMTFVVSGVLLLNYVAAILAGIIWPQAFNSNVMLLSHAILAFCLIFQTRELALANYAAAPSRQFFEFIWLLYYAEYFVYVFI

SEQ ID NO: 90 - protein, 309 aa, Artificial
  MPT21.20 (recombinant membrane-bound prenyltransferase)
MSDNSIATKILNFGHTCWKLQRPYVVKGMISIACGLFGKELLHNTNLISWGLMWKAFFALVPILSFNFFAAIMNQIYDVDIDRINKPDLPLVSGEMSIETAWILSIIVALTGLIVTIKLKSAPLFVFIYIFGIFAGFAYSVPPIRWKQNPSTNFLITISSHVGLAFTSYYASRAALGLPFELRPSFTFLLAFMTVMGMTIAFAKDISDIEGDAKYGVSTVATKLGARNMTFVVSGVLLLNYVAAILAGIIWPQAFNSNVMLLSHAILAFCLIFQTRELALANYAGAPSRQFFEFIWLLYYAEYFVYVFI

SEQ ID NO: 91 - protein, 309 aa, Artificial
  MPT21.22 (recombinant membrane-bound prenyltransferase)
MSDNSIATKILNFGHTCWKLQRPYVVKGMISIACGLFGKELLHNTNLISWGLMWKAFFALVPILSFNFFAAIMNQIYDVDIDRINKPDLPLVSGEMSIETAWILSIIVALTGLIVTIKLKSAPLFVFIYIFGIFAGFAYSVPPIRWKQNPSTNFLITISSHVGLAFTSYYASRAALGLPFELRPSFTFLLAFMTVMGMTIAFAKDISDIEGDAKYGVSTVATKLGARNMTFVVSGVLLLNYVAAILAGIIWPQAFNSNVMLLSHAILAFCLIFQTRELALANYASAPSRQFFEFLWLLYYAEYFVYVFI

SEQ ID NO: 92 - protein, 832 aa, Artificial
  GPS1.1-F11-PKC1.1-F11-MPT4, geranyl diphosphate synthase, polyketide cyclase, membrane-bound prenyltransferase fusion protein
MSKAKFESVFPRISEELVQLLRDEGLPQDAVQWFSDSLQYNCVGGKLNRGLSVVDTYQLLTGKKELDDEEYYRLALLGWLIELLQAFWLVSDDIMDESKTRRGQPCWYLKPKVGMIAIWDAFMLESGIYILLKKHFRQEKYYIDLVELFHDISFKTELGQLVDLLTAPEDEVDLNRFSLDKHSFIVRYKTAYYSFYLPVVLAMYVAGITNPKDLQQAMDVLIPLGEYFQVQDDYLDNFGDPEFIGKIGTDIQDNKCSWLVNKALQKATPEQRQILEDNYGVKDKSKELVIKKLYDDMKIEQDYLDYEEEVVGDIKKKIEQVDESRGFKKEVLNAFLAKIYKRQKGGAEAAAKEAAAKAGGSGGGSGGGGSGGSGGGGSGGGGSMAVKHLIVLKFKDEITEAQKEEFFKTFVNLVNIIPAMKDVYWGKDVTQKNKEEGYTHIVEVTFESVETIQDYIIHPAHVGFGDVYRSFWEKLLIFDYTPRKGGAEAAAKEAAAKAGGSGGGSGGGGSGGSGGGGSGGGGSMSDNSIATKILNFGHTCWKLQRPYVVKGMISIACGLFGRELFNNRHLFSWGLMWKAFFALVPILSFNFFAAIMNQIYDVDIDRINKPDLPLVSGEMSIETAWILSIIVALTGLIVTIKLKSAPLFVFIYIFGIFAGFAYSVPPIRWKQYPFTNFLITISSHVGLAFTSYSATTSALGLPFVWRPAFSFIIAFMTVMGMTIAFAKDISDIEGDAKYGVSTVATKLGARNMTFVVSGVLLLNYLVSISIGIIWPQVFKSNIMILSHAILAFCLIFQTRELALANYASAPSRQFFEFIWLLYYAEYFVYVFI

SEQ ID NO: 93 - protein, 832 aa, Artificial
  GPS1.1-F11-PKC1.1-F11-MPT4.1, geranyl diphosphate synthase, polyketide cyclase, point mutant membrane-bound prenyltransferase fusion protein
MSKAKFESVFPRISEELVQLLRDEGLPQDAVQWFSDSLQYNCVGGKLNRGLSVVDTYQLLTGKKELDDEEYYRLALLGWLIELLQAFWLVSDDIMDESKTRRGQPCWYLKPKVGMIAIWDAFMLESGIYILLKKHFRQEKYYIDLVELFHDISFKTELGQLVDLLTAPEDEVDLNRFSLDKHSFIVRYKTAYYSFYLPVVLAMYVAGITNPKDLQQAMDVLIPLGEYFQVQDDYLDNFGDPEFIGKIGTDIQDNKCSWLVNKALQKATPEQRQILEDNYGVKDKSKELVIKKLYDDMKIEQDYLDYEEEVVGDIKKKIEQVDESRGFKKEVLNAFLAKIYKRQKGGAEAAAKEAAAKAGGSGGGSGGGGSGGSGGGGSGGGGSMAVKHLIVLKFKDEITEAQKEEFFKTFVNLVNIIPAMKDVYWGKDVTQKNKEEGYTHIVEVTFESVETIQDYIIHPAHVGFGDVYRSFWEKLLIFDYTPRKGGAEAAAKEAAAKAGGSGGGSGGGGSGGSGGGGSGGGGSMSDNSIATKILNFGHTCWKLQRPYAVKGMISIACGLFGRELFNNRHLFSWGLMWKAFFALVPILSFNFFAAIMNQIYDVDIDRINKPDLPLVSGEMSIETAWILSIIVALTGLIVTIKLKSAPLFVFIYIFGIFAGFAYSVPPIRWKQYPFTNFLITISSHVGLAFTSYSATTSALGLPFVWRPAFSFIIAFMTVMGMTIAFAKDISDIEGDAKYGVSTVATKLGARNMTFVVSGVLLLNYLVSISIGIIWPQVFKSNIMILSHAILAFCLIFQTRELALANYASAPSRQFFEFIWLLYYAEYFVYVFI

SEQ ID NO: 94 - protein, 832 aa, Artificial
  GPS1.1-F11-PKC1.1-F11-MPT21.3, geranyl diphosphate synthase, polyketide cyclase, recombinant membrane-bound prenyltransferase fusion protein
MSKAKFESVFPRISEELVQLLRDEGLPQDAVQWFSDSLQYNCVGGKLNRGLSVVDTYQLLTGKKELDDEEYYRLALLGWLIELLQAFWLVSDDIMDESKTRRGQPCWYLKPKVGMIAIWDAFMLESGIYILLKKHFRQEKYYIDLVELFHDISFKTELGQLVDLLTAPEDEVDLNRFSLDKHSFIVRYKTAYYSFYLPVVLAMYVAGITNPKDLQQAMDVLIPLGEYFQVQDDYLDNFGDPEFIGKIGTDIQDNKCSWLVNKALQKATPEQRQILEDNYGVKDKSKELVIKKLYDDMKIEQDYLDYEEEVVGDIKKKIEQVDESRGFKKEVLNAFLAKIYKRQKGGAEAAAKEAAAKAGGSGGGSGGGGSGGSGGGGSGGGGSMAVKHLIVLKFKDEITEAQKEEFFKTFVNLVNIIPAMKDVYWGKDVTQKNKEEGYTHIVEVTFESVETIQDYIIHPAHVGFGDVYRSFWEKLLIFDYTPRKGGAEAAAKEAAAKAGGSGGGSGGGGSGGSGGGGSGGGGSMSDNSIATKILNFGHTCWKLQRPYAVKGMISIACGLFGKELLHNTNLISWGLMWKAFFALVPILSFNFFAAIMNQIYDVDIDRINKPDLPLVSGEMSIETAWILSIIVALTGLIVTIKLKSAPLFVFIYIFGIFAGFAYSVPPIRWKQNPSTNFLITISSHVGLAFTSYYASRAALGLPFELRPSFTFLLAFMTVMGMTIAFAKDISDIEGDAKYGVSTVATKLGARNMTFVVSGVLLLNYVAAILAGIIWPQAFNSNVMLLSHAILAFCLIFQTRELALANYASAPSRQFFEFIWLLYYAEYFVYVFI

SEQ ID NO: 95 - protein, 846 aa, Artificial
  GPS1.1-F11-PKC4.33-F11-MPT4.1, geranyl diphosphate synthase, polyketide cyclase, point mutant membrane-bound prenyltransferase fusion protein
MSKAKFESVFPRISEELVQLLRDEGLPQDAVQWFSDSLQYNCVGGKLNRGLSVVDTYQLLTGKKELDDEEYYRLALLGWLIELLQAFWLVSDDIMDESKTRRGQPCWYLKPKVGMIAIWDAFMLESGIYILLKKHFRQEKYYIDLVELFHDISFKTELGQLVDLLTAPEDEVDLNRFSLDKHSFIVRYKTAYYSFYLPVVLAMYVAGITNPKDLQQAMDVLIPLGEYFQVQDDYLDNFGDPEFIGKIGTDIQDNKCSWLVNKALQKATPEQRQILEDNYGVKDKSKELVIKKLYDDMKIEQDYLDYEEEVVGDIKKKIEQVDESRGFKKEVLNAFLAKIYKRQKGGAEAAAKEAAAKAGGSGGGSGGGGSGGSGGGGSGGGGSMGEANKGVVKHVIILKFKEGITEAQKEEMFKTYVNLVNLVPAMKAVQWGKLEVNNKLGNGGYTHIVESTFESVETIQDYIIHPAHVGFGDVYRSFWEKLLIFDYTPTIVLPNSSYGGAEAAAKEAAAKAGGSGGGSGGGGSGGSGGGGSGGGGSMSDNSIATKILNFGHTCWKLQRPYAVKGMISIACGLFGRELFNNRHLFSWGLMWKAFFALVPILSFNFFAAIMNQIYDVDIDRINKPDLPLVSGEMSIETAWILSIIVALTGLIVTIKLKSAPLFVFIYIFGIFAGFAYSVPPIRWKQYPFTNFLITISSHVGLAFTSYSATTSALGLPFVWRPAFSFIIAFMTVMGMTIAFAKDISDIEGDAKYGVSTVATKLGARNMTFVVSGVLLLNYLVSISIGIIWPQVFKSNIMILSHAILAFCLIFQTRELALANYASAPSRQFFEFIWLLYYAEYFVYVFI

SEQ ID NO: 96 - protein, 846 aa, Artificial
  GPS1.1-F11-PKC4.33-F11-MPT21.3, geranyl diphosphate synthase, polyketide cyclase, recombinant membrane-bound prenyltransferase fusion protein
MSKAKFESVFPRISEELVQLLRDEGLPQDAVQWFSDSLQYNCVGGKLNRGLSVVDTYQLLTGKKELDDEEYYRLALLGWLIELLQAFWLVSDDIMDESKTRRGQPCWYLKPKVGMIAIWDAFMLESGIYILLKKHFRQEKYYIDLVELFHDISFKTELGQLVDLLTAPEDEVDLNRFSLDKHSFIVRYKTAYYSFYLPVVLAMYVAGITNPKDLQQAMDVLIPLGEYFQVQDDYLDNFGDPEFIGKIGTDIQDNKCSWLVNKALQKATPEQRQILEDNYGVKDKSKELVIKKLYDDMKIEQDYLDYEEEVVGDIKKKIEQVDESRGFKKEVLNAFLAKIYKRQKGGAEAAAKEAAAKAGGSGGGSGGGGSGGSGGGGSGGGGSMGEANKGVVKHVIILKFKEGITEAQKEEMFKTYVNLVNLVPAMKAVQWGKLEVNNKLGNGGYTHIVESTFESVETIQDYIIHPAHVGFGDVYRSFWEKLLIFDYTPTIVLPNSSYGGAEAAAKEAAAKAGGSGGGSGGGGSGGSGGGGSGGGGSMSDNSIATKILNFGHTCWKLQRPYAVKGMISIACGLFGKELLHNTNLISWGLMWKAFFALVPILSFNFFAAIMNQIYDVDIDRINKPDLPLVSGEMSIETAWILSIIVALTGLIVTIKLKSAPLFVFIYIFGIFAGFAYSVPPIRWKQNPSTNFLITISSHVGLAFTSYYASRAALGLPFELRPSFTFLLAFMTVMGMTIAFAKDISDIEGDAKYGVSTVATKLGARNMTFVVSGVLLLNYVAAILAGIIWPQAFNSNVMLLSHAILAFCLIFQTRELALANYASAPSRQFFEFIWLLYYAEYFVYVFI

SEQ ID NO: 97 - protein, 768 aa, Artificial
  GPS1.1-PKC4.33-MPT4.1, geranyl diphosphate synthase, polyketide cyclase, point mutant membrane-bound prenyltransferase fusion protein
MSKAKFESVFPRISEELVQLLRDEGLPQDAVQWFSDSLQYNCVGGKLNRGLSVVDTYQLLTGKKELDDEEYYRLALLGWLIELLQAFWLVSDDIMDESKTRRGQPCWYLKPKVGMIAIWDAFMLESGIYILLKKHFRQEKYYIDLVELFHDISFKTELGQLVDLLTAPEDEVDLNRFSLDKHSFIVRYKTAYYSFYLPVVLAMYVAGITNPKDLQQAMDVLIPLGEYFQVQDDYLDNFGDPEFIGKIGTDIQDNKCSWLVNKALQKATPEQRQILEDNYGVKDKSKELVIKKLYDDMKIEQDYLDYEEEVVGDIKKKIEQVDESRGFKKEVLNAFLAKIYKRQKMGEANKGVVKHVIILKFKEGITEAQKEEMFKTYVNLVNLVPAMKAVQWGKLEVNNKLGNGGYTHIVESTFESVETIQDYIIHPAHVGFGDVYRSFWEKLLIFDYTPTIVLPNSSYMSDNSIATKILNFGHTCWKLQRPYAVKGMISIACGLFGRELFNNRHLFSWGLMWKAFFALVPILSFNFFAAIMNQIYDVDIDRINKPDLPLVSGEMSIETAWILSIIVALTGLIVTIKLKSAPLFVFIYIFGIFAGFAYSVPPIRWKQYPFTNFLITISSHVGLAFTSYSATTSALGLPFVWRPAFSFIIAFMTVMGMTIAFAKDISDIEGDAKYGVSTVATKLGARNMTFVVSGVLLLNYLVSISIGIIWPQVFKSNIMILSHAILAFCLIFQTRELALANYASAPSRQFFEFIWLLYYAEYFVYVFI

SEQ ID NO: 98 - protein, 768 aa, Artificial
  GPS1.1-PKC4.33-MPT21.3, geranyl diphosphate synthase, polyketide cyclase, recombinant membrane-bound prenyltransferase fusion protein
MSKAKFESVFPRISEELVQLLRDEGLPQDAVQWFSDSLQYNCVGGKLNRGLSVVDTYQLLTGKKELDDEEYYRLALLGWLIELLQAFWLVSDDIMDESKTRRGQPCWYLKPKVGMIAIWDAFMLESGIYILLKKHFRQEKYYIDLVELFHDISFKTELGQLVDLLTAPEDEVDLNRFSLDKHSFIVRYKTAYYSFYLPVVLAMYVAGITNPKDLQQAMDVLIPLGEYFQVQDDYLDNFGDPEFIGKIGTDIQDNKCSWLVNKALQKATPEQRQILEDNYGVKDKSKELVIKKLYDDMKIEQDYLDYEEEVVGDIKKKIEQVDESRGFKKEVLNAFLAKIYKRQKMGEANKGVVKHVIILKFKEGITEAQKEEMFKTYVNLVNLVPAMKAVQWGKLEVNNKLGNGGYTHIVESTFESVETIQDYIIHPAHVGFGDVYRSFWEKLLIFDYTPTIVLPNSSYMSDNSIATKILNFGHTCWKLQRPYAVKGMISIACGLFGKELLHNTNLISWGLMWKAFFALVPILSFNFFAAIMNQIYDVDIDRINKPDLPLVSGEMSIETAWILSIIVALTGLIVTIKLKSAPLFVFIYIFGIFAGFAYSVPPIRWKQNPSTNFLITISSHVGLAFTSYYASRAALGLPFELRPSFTFLLAFMTVMGMTIAFAKDISDIEGDAKYGVSTVATKLGARNMTFVVSGVLLLNYVAAILAGIIWPQAFNSNVMLLSHAILAFCLIFQTRELALANYASAPSRQFFEFIWLLYYAEYFVYVFI

SEQ ID NO: 99 - protein, 832 aa, Artificial
  PKC1.1-F11-GPS1.1-F11-MPT4.1, geranyl diphosphate synthase, polyketide cyclase, point mutant membrane-bound prenyltransferase fusion protein
MAVKHLIVLKFKDEITEAQKEEFFKTFVNLVNIIPAMKDVYWGKDVTQKNKEEGYTHIVEVTFESVETIQDYIIHPAHVGFGDVYRSFWEKLLIFDYTPRKGGAEAAAKEAAAKAGGSGGGSGGGGSGGSGGGGSGGGGSMSKAKFESVFPRISEELVQLLRDEGLPQDAVQWFSDSLQYNCVGGKLNRGLSVVDTYQLLTGKKELDDEEYYRLALLGWLIELLQAFWLVSDDIMDESKTRRGQPCWYLKPKVGMIAIWDAFMLESGIYILLKKHFRQEKYYIDLVELFHDISFKTELGQLVDLLTAPEDEVDLNRFSLDKHSFIVRYKTAYYSFYLPVVLAMYVAGITNPKDLQQAMDVLIPLGEYFQVQDDYLDNFGDPEFIGKIGTDIQDNKCSWLVNKALQKATPEQRQILEDNYGVKDKSKELVIKKLYDDMKIEQDYLDYEEEVVGDIKKKIEQVDESRGFKKEVLNAFLAKIYKRQKGGAEAAAKEAAAKAGGSGGGSGGGGSGGSGGGGSGGGGSMSDNSIATKILNFGHTCWKLQRPYAVKGMISIACGLFGRELFNNRHLFSWGLMWKAFFALVPILSFNFFAAIMNQIYDVDIDRINKPDLPLVSGEMSIETAWILSIIVALTGLIVTIKLKSAPLFVFIYIFGIFAGFAYSVPPIRWKQYPFTNFLITISSHVGLAFTSYSATTSALGLPFVWRPAFSFIIAFMTVMGMTIAFAKDISDIEGDAKYGVSTVATKLGARNMTFVVSGVLLLNYLVSISIGIIWPQVFKSNIMILSHAILAFCLIFQTRELALANYASAPSRQFFEFIWLLYYAEYFVYVFI

SEQ ID NO: 100 - protein, 846 aa, Artificial
  PKC4.33-F11-GPS1.1-F11-MPT4.1, polyketide cyclase, geranyl diphosphate synthase, point mutant prenyltransferase fusion protein
MGEANKGVVKHVIILKFKEGITEAQKEEMFKTYVNLVNLVPAMKAVQWGKLEVNNKLGNGGYTHIVESTFESVETIQDYIIHPAHVGFGDVYRSFWEKLLIFDYTPTIVLPNSSYGGAEAAAKEAAAKAGGSGGGSGGGGSGGSGGGGSGGGGSMSKAKFESVFPRISEELVQLLRDEGLPQDAVQWFSDSLQYNCVGGKLNRGLSVVDTYQLLTGKKELDDEEYYRLALLGWLIELLQAFWLVSDDIMDESKTRRGQPCWYLKPKVGMIAIWDAFMLESGIYILLKKHFRQEKYYIDLVELFHDISFKTELGQLVDLLTAPEDEVDLNRFSLDKHSFIVRYKTAYYSFYLPVVLAMYVAGITNPKDLQQAMDVLIPLGEYFQVQDDYLDNFGDPEFIGKIGTDIQDNKCSWLVNKALQKATPEQRQILEDNYGVKDKSKELVIKKLYDDMKIEQDYLDYEEEVVGDIKKKIEQVDESRGFKKEVLNAFLAKIYKRQKGGAEAAAKEAAAKAGGSGGGSGGGGSGGSGGGGSGGGGSMSDNSIATKILNFGHTCWKLQRPYAVKGMISIACGLFGRELFNNRHLFSWGLMWKAFFALVPILSFNFFAAIMNQIYDVDIDRINKPDLPLVSGEMSIETAWILSIIVALTGLIVTIKLKSAPLFVFIYIFGIFAGFAYSVPPIRWKQYPFTNFLITISSHVGLAFTSYSATTSALGLPFVWRPAFSFIIAFMTVMGMTIAFAKDISDIEGDAKYGVSTVATKLGARNMTFVVSGVLLLNYLVSISIGIIWPQVFKSNIMILSHAILAFCLIFQTRELALANYASAPSRQFFEFIWLLYYAEYFVYVFI

SEQ ID NO: 101 - protein, 807 aa, Artificial
  PKC4.33-GPS1.1-F11-MPT4.1, polyketide cyclase, geranyl diphosphate synthase, point mutant prenyltransferase fusion protein
MGEANKGVVKHVIILKFKEGITEAQKEEMFKTYVNLVNLVPAMKAVQWGKLEVNNKLGNGGYTHIVESTFESVETIQDYIIHPAHVGFGDVYRSFWEKLLIFDYTPTIVLPNSSYMSKAKFESVFPRISEELVQLLRDEGLPQDAVQWFSDSLQYNCVGGKLNRGLSVVDTYQLLTGKKELDDEEYYRLALLGWLIELLQAFWLVSDDIMDESKTRRGQPCWYLKPKVGMIAIWDAFMLESGIYILLKKHFRQEKYYIDLVELFHDISFKTELGQLVDLLTAPEDEVDLNRFSLDKHSFIVRYKTAYYSFYLPVVLAMYVAGITNPKDLQQAMDVLIPLGEYFQVQDDYLDNFGDPEFIGKIGTDIQDNKCSWLVNKALQKATPEQRQILEDNYGVKDKSKELVIKKLYDDMKIEQDYLDYEEEVVGDIKKKIEQVDESRGFKKEVLNAFLAKIYKRQKGGAEAAAKEAAAKAGGSGGGSGGGGSGGSGGGGSGGGGSMSDNSIATKILNFGHTCWKLQRPYAVKGMISIACGLFGRELFNNRHLFSWGLMWKAFFALVPILSFNFFAAIMNQIYDVDIDRINKPDLPLVSGEMSIETAWILSIIVALTGLIVTIKLKSAPLFVFIYIFGIFAGFAYSVPPIRWKQYPFTNFLITISSHVGLAFTSYSATTSALGLPFVWRPAFSFIIAFMTVMGMTIAFAKDISDIEGDAKYGVSTVATKLGARNMTFVVSGVLLLNYLVSISIGIIWPQVFKSNIMILSHAILAFCLIFQTRELALANYASAPSRQFFEFIWLLYYAEYFVYVFI

SEQ ID NO: 102 - protein, 832 aa, Artificial
  PKC1.1-F11-GPS1.1-F11-MPT21.3, polyketide cyclase, geranyl diphosphate synthase, recombinant membrane-bound prenyltransferase fusion protein
MAVKHLIVLKFKDEITEAQKEEFFKTFVNLVNIIPAMKDVYWGKDVTQKNKEEGYTHIVEVTFESVETIQDYIIHPAHVGFGDVYRSFWEKLLIFDYTPRKGGAEAAAKEAAAKAGGSGGGSGGGGSGGSGGGGSGGGGSMSKAKFESVFPRISEELVQLLRDEGLPQDAVQWFSDSLQYNCVGGKLNRGLSVVDTYQLLTGKKELDDEEYYRLALLGWLIELLQAFWLVSDDIMDESKTRRGQPCWYLKPKVGMIAIWDAFMLESGIYILLKKHFRQEKYYIDLVELFHDISFKTELGQLVDLLTAPEDEVDLNRFSLDKHSFIVRYKTAYYSFYLPVVLAMYVAGITNPKDLQQAMDVLIPLGEYFQVQDDYLDNFGDPEFIGKIGTDIQDNKCSWLVNKALQKATPEQRQILEDNYGVKDKSKELVIKKLYDDMKIEQDYLDYEEEVVGDIKKKIEQVDESRGFKKEVLNAFLAKIYKRQKGGAEAAAKEAAAKAGGSGGGSGGGGSGGSGGGGSGGGGSMSDNSIATKILNFGHTCWKLQRPYAVKGMISIACGLFGKELLHNTNLISWGLMWKAFFALVPILSFNFFAAIMNQIYDVDIDRINKPDLPLVSGEMSIETAWILSIIVALTGLIVTIKLKSAPLFVFIYIFGIFAGFAYSVPPIRWKQNPSTNFLITISSHVGLAFTSYYASRAALGLPFELRPSFTFLLAFMTVMGMTIAFAKDISDIEGDAKYGVSTVATKLGARNMTFVVSGVLLLNYVAAILAGIIWPQAFNSNVMLLSHAILAFCLIFQTRELALANYASAPSRQFFEFIWLLYYAEYFVYVFI

SEQ ID NO: 103 - protein, 846 aa, Artificial
  PKC4.33-F11-GPS1.1-F11-MPT21.3, polyketide cyclase, geranyl diphosphate synthase, recombinant membrane-bound prenyltransferase fusion protein
MGEANKGVVKHVIILKFKEGITEAQKEEMFKTYVNLVNLVPAMKAVQWGKLEVNNKLGNGGYTHIVESTFESVETIQDYIIHPAHVGFGDVYRSFWEKLLIFDYTPTIVLPNSSYGGAEAAAKEAAAKAGGSGGGSGGGGSGGSGGGGSGGGGSMSKAKFESVFPRISEELVQLLRDEGLPQDAVQWFSDSLQYNCVGGKLNRGLSVVDTYQLLTGKKELDDEEYYRLALLGWLIELLQAFWLVSDDIMDESKTRRGQPCWYLKPKVGMIAIWDAFMLESGIYILLKKHFRQEKYYIDLVELFHDISFKTELGQLVDLLTAPEDEVDLNRFSLDKHSFIVRYKTAYYSFYLPVVLAMYVAGITNPKDLQQAMDVLIPLGEYFQVQDDYLDNFGDPEFIGKIGTDIQDNKCSWLVNKALQKATPEQRQILEDNYGVKDKSKELVIKKLYDDMKIEQDYLDYEEEVVGDIKKKIEQVDESRGFKKEVLNAFLAKIYKRQKGGAEAAAKEAAAKAGGSGGGSGGGGSGGSGGGGSGGGGSMSDNSIATKILNFGHTCWKLQRPYAVKGMISIACGLFGKELLHNTNLISWGLMWKAFFALVPILSFNFFAAIMNQIYDVDIDRINKPDLPLVSGEMSIETAWILSIIVALTGLIVTIKLKSAPLFVFIYIFGIFAGFAYSVPPIRWKQNPSTNFLITISSHVGLAFTSYYASRAALGLPFELRPSFTFLLAFMTVMGMTIAFAKDISDIEGDAKYGVSTVATKLGARNMTFVVSGVLLLNYVAAILAGIIWPQAFNSNVMLLSHAILAFCLIFQTRELALANYASAPSRQFFEFIWLLYYAEYFVYVFI

SEQ ID NO: 104 - protein, 807 aa, Artificial
  PKC4.33-GPS1.1-F11-MPT21.3, polyketide cyclase, geranyl diphosphate synthase, recombinant membrane-bound prenyltransferase fusion protein
MGEANKGVVKHVIILKFKEGITEAQKEEMFKTYVNLVNLVPAMKAVQWGKLEVNNKLGNGGYTHIVESTFESVETIQDYIIHPAHVGFGDVYRSFWEKLLIFDYTPTIVLPNSSYMSKAKFESVFPRISEELVQLLRDEGLPQDAVQWFSDSLQYNCVGGKLNRGLSVVDTYQLLTGKKELDDEEYYRLALLGWLIELLQAFWLVSDDIMDESKTRRGQPCWYLKPKVGMIAIWDAFMLESGIYILLKKHFRQEKYYIDLVELFHDISFKTELGQLVDLLTAPEDEVDLNRFSLDKHSFIVRYKTAYYSFYLPVVLAMYVAGITNPKDLQQAMDVLIPLGEYFQVQDDYLDNFGDPEFIGKIGTDIQDNKCSWLVNKALQKATPEQRQILEDNYGVKDKSKELVIKKLYDDMKIEQDYLDYEEEVVGDIKKKIEQVDESRGFKKEVLNAFLAKIYKRQKGGAEAAAKEAAAKAGGSGGGSGGGGSGGSGGGGSGGGGSMSDNSIATKILNFGHTCWKLQRPYAVKGMISIACGLFGKELLHNTNLISWGLMWKAFFALVPILSFNFFAAIMNQIYDVDIDRINKPDLPLVSGEMSIETAWILSIIVALTGLIVTIKLKSAPLFVFIYIFGIFAGFAYSVPPIRWKQNPSTNFLITISSHVGLAFTSYYASRAALGLPFELRPSFTFLLAFMTVMGMTIAFAKDISDIEGDAKYGVSTVATKLGARNMTFVVSGVLLLNYVAAILAGIIWPQAFNSNVMLLSHAILAFCLIFQTRELALANYASAPSRQFFEFIWLLYYAEYFVYVFI

SEQ ID NO: 105 - protein, 660 aa, Unknown
  HCS2 (hexanoyl-CoA synthetase)
MASEENDLVFPSKEFSGQALVSSPQQYMEMHKRSMDDPAAFWSDIASEFYWKQKWGDQVFSENLDVRKGPISIEWFKGGITNICYNCLDKNVEAGLGDKTAIHWEGNELGVDASLTYSELLQRVCQLANYLKDNGVKKGDAVVIYLPMLMELPIAMLACARIGAVHSVVFAGFSADSLAQRIVDCKPNVILTCNAVKRGPKTINLKAIVDAALDQSSKDGVSVGICLTYDNSLATTRENTKWQNGRDVWWQDVISQYPTSCEVEWVDAEDPLFLLYTSGSTGKPKGVLHTTGGYMIYTATTFKYAFDYKSTDVYWCTADCGWIGGHSYVTYGPMLNGATVVVFEGAPNYPDPGRCWDIVDKYKVSIFYTAPTLVRSLMRDDDKFVTRHSRKSLRVLGSAGEPINPSAWRWFFNVVGDSRCPISDTWGQTETGGFMITPLPGAWPQKPGSATFPFFGVQPVIVDEKGNEIEGECSGYLCVKGSWPGAFRTLFGDHERYETTYFKPFAGYYFSGDGCSRDKDGYYWLTGRVDDVINVSGHRIGTAEVESALVLHPQCAEAAVVGIEHEVKGQGIYAFVTLLEGVPYSEELRKSLVLMVRNQIGAFAAPDRIHWAPGLPKTRSGKIMRRILRKIASRQLEELGDTSTLADPSVVDQLIALADV

SEQ ID NO: 106 - protein, 101 aa, Unknown
  PKC1.0 (polyketide cyclase)
MAVKHLIVLKFKDEITEAQKEEFFKTYVNLVNIIPAMKDVYWGKDVTQKNKEEGYTHIVEVTFESVETIQDYIIHPAHVGFGDVYRSFWEKLLIFDYTPRK

SEQ ID NO: 107 - protein, 101 aa, Unknown
  PKC1.1 (polyketide cyclase)
MAVKHLIVLKFKDEITEAQKEEFFKTFVNLVNIIPAMKDVYWGKDVTQKNKEEGYTHIVEVTFESVETIQDYIIHPAHVGFGDVYRSFWEKLLIFDYTPRK

SEQ ID NO: 108 - protein, 115 aa, Unknown
  PKC4.33 (polyketide cyclase)
MGEANKGVVKHVIILKFKEGITEAQKEEMFKTYVNLVNLVPAMKAVQWGKLEVNNKLGNGGYTHIVESTFESVETIQDYIIHPAHVGFGDVYRSFWEKLLIFDYTPTIVLPNSSY

SEQ ID NO: 109 - protein, 102 aa, Unknown
  PKC11 (polyketide cyclase)
MAVKHLIVLKFKDEITEAQKEEFFKTYVNLVNIIPAMKDVYWGKLEVNNKLGNGGYTHIVEVTFESVETIQDYIIHPAHVGFGDVYRSFWEKLLIFDYTPRK

SEQ ID NO: 110 - protein, 817 aa, Artificial
  GPS1.1-F11-PKC1.1-F18-APT73.77, geranyl diphosphate synthase, polyketide cyclase, mutant soluble aromatic prentyltransfeerase fusion protein
MSKAKFESVFPRISEELVQLLRDEGLPQDAVQWFSDSLQYNCVGGKLNRGLSVVDTYQLLTGKKELDDEEYYRLALLGWLIELLQAFWLVSDDIMDESKTRRGQPCWYLKPKVGMIAIWDAFMLESGIYILLKKHFRQEKYYIDLVELFHDISFKTELGQLVDLLTAPEDEVDLNRFSLDKHSFIVRYKTAYYSFYLPVVLAMYVAGITNPKDLQQAMDVLIPLGEYFQVQDDYLDNFGDPEFIGKIGTDIQDNKCSWLVNKALQKATPEQRQILEDNYGVKDKSKELVIKKLYDDMKIEQDYLDYEEEVVGDIKKKIEQVDESRGFKKEVLNAFLAKIYKRQKGGAEAAAKEAAAKAGGSGGGSGGGGSGGSGGGGSGGGGSMAVKHLIVLKFKDEITEAQKEEFFKTFVNLVNIIPAMKDVYWGKDVTQKNKEEGYTHIVEVTFESVETIQDYIIHPAHVGFGDVYRSFWEKLLIFDYTPRKGGGGSLEDPAVWEAGKVVAKGVGTADITATTSNGLIASSEEADNAATSMDEVYAAVEQTSRLLDVPCSPDRFEPVWKAFGDQLPDSHLVFSMAAGEAHRGELDFDFSLRPEGADPYTTALEHGFIEPTDHPVGSVLAEVGKRFAIASYGVEYGVVGGFKKSYAFFPLDDFPPLAQFAEVPSVPPCLAGHVETLTRLGFDDKVSIIGVNYRKNTLNVYLAASAVDTGDKLALLRAFGYPEPDARVRQFIERSFRLYPTFNWDSSAAERICFAVHTQQPGELPAPHDEPTEAFARQVPHVYEGGREFVSGVALAPSGASYYKLAALYQKARRCLH

SEQ ID NO: 111 - protein, 831 aa, Artificial
  GPS1.1-F11-PKC4.33-F18-APT73.77, geranyl diphosphate synthase, polyketide cyclase, mutant soluble aromatic prenyltransferase fusion protein
MSKAKFESVFPRISEELVQLLRDEGLPQDAVQWFSDSLQYNCVGGKLNRGLSVVDTYQLLTGKKELDDEEYYRLALLGWLIELLQAFWLVSDDIMDESKTRRGQPCWYLKPKVGMIAIWDAFMLESGIYILLKKHFRQEKYYIDLVELFHDISFKTELGQLVDLLTAPEDEVDLNRFSLDKHSFIVRYKTAYYSFYLPVVLAMYVAGITNPKDLQQAMDVLIPLGEYFQVQDDYLDNFGDPEFIGKIGTDIQDNKCSWLVNKALQKATPEQRQILEDNYGVKDKSKELVIKKLYDDMKIEQDYLDYEEEVVGDIKKKIEQVDESRGFKKEVLNAFLAKIYKRQKGGAEAAAKEAAAKAGGSGGGSGGGGSGGSGGGGSGGGGSMGEANKGVVKHVIILKFKEGITEAQKEEMFKTYVNLVNLVPAMKAVQWGKLEVNNKLGNGGYTHIVESTFESVETIQDYIIHPAHVGFGDVYRSFWEKLLIFDYTPTIVLPNSSYGGGGSLEDPAVWEAGKVVAKGVGTADITATTSNGLIASSEEADNAATSMDEVYAAVEQTSRLLDVPCSPDRFEPVWKAFGDQLPDSHLVFSMAAGEAHRGELDFDFSLRPEGADPYTTALEHGFIEPTDHPVGSVLAEVGKRFAIASYGVEYGVVGGFKKSYAFFPLDDFPPLAQFAEVPSVPPCLAGHVETLTRLGFDDKVSIIGVNYRKNTLNVYLAASAVDTGDKLALLRAFGYPEPDARVRQFIERSFRLYPTFNWDSSAAERICFAVHTQQPGELPAPHDEPTEAFARQVPHVYEGGREFVSGVALAPSGASYYKLAALYQKARRCLH

SEQ ID NO: 112 - protein, 744 aa, Artificial
  GPS1.1-PKC4.33-APT73.77, geranyl diphosphate synthase, polyketide cyclase, mutant soluble aromatic prenyltransferase fusion protein
MSKAKFESVFPRISEELVQLLRDEGLPQDAVQWFSDSLQYNCVGGKLNRGLSVVDTYQLLTGKKELDDEEYYRLALLGWLIELLQAFWLVSDDIMDESKTRRGQPCWYLKPKVGMIAIWDAFMLESGIYILLKKHFRQEKYYIDLVELFHDISFKTELGQLVDLLTAPEDEVDLNRFSLDKHSFIVRYKTAYYSFYLPVVLAMYVAGITNPKDLQQAMDVLIPLGEYFQVQDDYLDNFGDPEFIGKIGTDIQDNKCSWLVNKALQKATPEQRQILEDNYGVKDKSKELVIKKLYDDMKIEQDYLDYEEEVVGDIKKKIEQVDESRGFKKEVLNAFLAKIYKRQKMGEANKGVVKHVIILKFKEGITEAQKEEMFKTYVNLVNLVPAMKAVQWGKLEVNNKLGNGGYTHIVESTFESVETIQDYIIHPAHVGFGDVYRSFWEKLLIFDYTPTIVLPNSSYMDEVYAAVEQTSRLLDVPCSPDRFEPVWKAFGDQLPDSHLVFSMAAGEAHRGELDFDFSLRPEGADPYTTALEHGFIEPTDHPVGSVLAEVGKRFAIASYGVEYGVVGGFKKSYAFFPLDDFPPLAQFAEVPSVPPCLAGHVETLTRLGFDDKVSIIGVNYRKNTLNVYLAASAVDTGDKLALLRAFGYPEPDARVRQFIERSFRLYPTFNWDSSAAERICFAVHTQQPGELPAPHDEPTEAFARQVPHVYEGGREFVSGVALAPSGASYYKLAALYQKARRCLH

SEQ ID NO: 113 - protein, 833 aa, Artificial
  GPS1.1-F11-PKC11-F11-MPT4, geranyl diphosphate synthase, polyketide cyclase, membrane-bound prenyltransferase fusion protein
MSKAKFESVFPRISEELVQLLRDEGLPQDAVQWFSDSLQYNCVGGKLNRGLSVVDTYQLLTGKKELDDEEYYRLALLGWLIELLQAFWLVSDDIMDESKTRRGQPCWYLKPKVGMIAIWDAFMLESGIYILLKKHFRQEKYYIDLVELFHDISFKTELGQLVDLLTAPEDEVDLNRFSLDKHSFIVRYKTAYYSFYLPVVLAMYVAGITNPKDLQQAMDVLIPLGEYFQVQDDYLDNFGDPEFIGKIGTDIQDNKCSWLVNKALQKATPEQRQILEDNYGVKDKSKELVIKKLYDDMKIEQDYLDYEEEVVGDIKKKIEQVDESRGFKKEVLNAFLAKIYKRQKGGAEAAAKEAAAKAGGSGGGSGGGGSGGSGGGGSGGGGSMAVKHLIVLKFKDEITEAQKEEFFKTYVNLVNIIPAMKDVYWGKLEVNNKLGNGGYTHIVEVTFESVETIQDYIIHPAHVGFGDVYRSFWEKLLIFDYTPRKGGAEAAAKEAAAKAGGSGGGSGGGGSGGSGGGGSGGGGSMSDNSIATKILNFGHTCWKLQRPYVVKGMISIACGLFGRELFNNRHLFSWGLMWKAFFALVPILSFNFFAAIMNQIYDVDIDRINKPDLPLVSGEMSIETAWILSIIVALTGLIVTIKLKSAPLFVFIYIFGIFAGFAYSVPPIRWKQYPFTNFLITISSHVGLAFTSYSATTSALGLPFVWRPAFSFIIAFMTVMGMTIAFAKDISDIEGDAKYGVSTVATKLGARNMTFVVSGVLLLNYLVSISIGIIWPQVFKSNIMILSHAILAFCLIFQTRELALANYASAPSRQFFEFIWLLYYAEYFVYVFI

SEQ ID NO: 114 - protein, 833 aa, Artificial
  GPS1.1-F11-PKC11-F11-MPT4.1, geranyl diphosphate synthase, polyketide cyclase, point mutant membrane-bound, prenyltransferase fusion protein
MSKAKFESVFPRISEELVQLLRDEGLPQDAVQWFSDSLQYNCVGGKLNRGLSVVDTYQLLTGKKELDDEEYYRLALLGWLIELLQAFWLVSDDIMDESKTRRGQPCWYLKPKVGMIAIWDAFMLESGIYILLKKHFRQEKYYIDLVELFHDISFKTELGQLVDLLTAPEDEVDLNRFSLDKHSFIVRYKTAYYSFYLPVVLAMYVAGITNPKDLQQAMDVLIPLGEYFQVQDDYLDNFGDPEFIGKIGTDIQDNKCSWLVNKALQKATPEQRQILEDNYGVKDKSKELVIKKLYDDMKIEQDYLDYEEEVVGDIKKKIEQVDESRGFKKEVLNAFLAKIYKRQKGGAEAAAKEAAAKAGGSGGGSGGGGSGGSGGGGSGGGGSMAVKHLIVLKFKDEITEAQKEEFFKTYVNLVNIIPAMKDVYWGKLEVNNKLGNGGYTHIVEVTFESVETIQDYIIHPAHVGFGDVYRSFWEKLLIFDYTPRKGGAEAAAKEAAAKAGGSGGGSGGGGSGGSGGGGSGGGGSMSDNSIATKILNFGHTCWKLQRPYAVKGMISIACGLFGRELFNNRHLFSWGLMWKAFFALVPILSFNFFAAIMNQIYDVDIDRINKPDLPLVSGEMSIETAWILSIIVALTGLIVTIKLKSAPLFVFIYIFGIFAGFAYSVPPIRWKQYPFTNFLITISSHVGLAFTSYSATTSALGLPFVWRPAFSFIIAFMTVMGMTIAFAKDISDIEGDAKYGVSTVATKLGARNMTFVVSGVLLLNYLVSISIGIIWPQVFKSNIMILSHAILAFCLIFQTRELALANYASAPSRQFFEFIWLLYYAEYFVYVFI